(19) United States
(12) Patent Application Publication
(10) Pub. No.: US 2024/0099950 A1
(43) Pub. Date: Mar. 28, 2024

(54) COSMETIC COMPOSITIONS AND METHODS OF USE THEREOF

(71) Applicant: Amyris, Inc., Emeryville, CA (US)

(72) Inventors: Diva CHAN, Emeryville, CA (US); Ro OTERI, Emeryville, CA (US); Ashlee NUNES, Emeryville, CA (US)

(73) Assignee: Amyris, Inc., Emeryville, CA (US)

(21) Appl. No.: 18/271,721

(22) PCT Filed: Jan. 11, 2022
(86) PCT No.: PCT/US2022/011973

§ 371 Date: Jul. 11, 2023

Related U.S. Application Data
(60) Provisional application No. 63/166,076, filed on Mar. 25, 2021.
(60) Provisional application No. 63/136,151, filed on Jan. 11, 2021.

(51) Int. Cl. A61K 8/34; A61K 8/31; A61K 8/37; A61K 8/92; A61K 8/9789; A61Q 19/00

(52) U.S. Cl. CPC A61K 8/347; A61K 8/31; A61K 8/375; A61K 8/922; A61K 8/9789; A61Q 19/00; A61K 2800/805

(57) ABSTRACT

The disclosure provides cosmetic compositions including cannabigerol (CBG), as well as methods of using the same for reducing skin redness, improving skin barrier functionality, brightening the skin of a subject, and enhancing the outward appearance of the skin of a subject (e.g., a male or female human subject).

### BACKGROUND OF THE INVENTION

Cosmetics may be used to improve a person's appearance. For example, a person may apply lotions to promote the skin's condition and hydration, and may also apply short-term cosmetic formulations, such as concealer or foundation, to hide imperfections such as redness, dryness, and dullness. However, there remains a need for new compositions and methods for improving the outward appearance of skin and reducing the appearance of skin imperfections.

### SUMMARY OF THE INVENTION

The present disclosure provides compositions (e.g., cosmetic compositions) that contain cannabigerol (CBG). The cosmetic compositions of the disclosure may further contain capric/caprylic triglycerides (CCT) and/or squalane. For example, the cosmetic compositions described herein may contain CBG in combination with CCT and, optionally, one or more carriers, diluents, or excipients. In some embodiments, the cosmetic compositions of the disclosure contain CBG in combination with squalane and, optionally, one or more carriers, diluents, or excipients. The cosmetic compositions described herein may further contain additional substances, such as one or more additional cannabinoids. For example, the cosmetic compositions of the disclosure may further include cannabidiol (CBD). The cosmetics compositions of the disclosure may also contain one or more essential oils and/or plant extracts Additional examples of substances that may further be included in the cosmetic compositions of the disclosure are provided in the sections that follow.

The cosmetic compositions of the disclosure can be used to engender a series of beneficial results for the end user. Particularly, the cosmetic compositions described herein can be administered to a subject (e.g., a male or female human subject) so as to improve the quality and/or the outward appearance of the subject's skin. Without being limited by mechanism, it has presently been discovered that cosmetic compositions containing CBG can be used to improve skin hydration by enhancing the barrier functionality of a subject's skin, thereby reducing transepidermal water loss. Cosmetic compositions containing CBG may also reduce redness in a subject's skin, such as redness due to inflammation caused by one or more of a variety of underlying conditions. Additionally, cosmetic compositions containing CBG may hide one or more imperfections in a subject's skin, such as a blemish caused by lack of hydration or chronic or acute inflammation. Cosmetic compositions containing CBG may also be used to brighten a subject's skin, for example, by increasing skin luminosity and/or improving the uniformity of a subject's skin tone. These examples illustrate some of the ways in which the compositions (e.g., cosmetic compositions) of the disclosure may improve the outward appearance of a subject's skin. Further examples are provided herein.

In a first aspect, the disclosure features a cosmetic composition containing CBG. In some embodiments, the composition further contains CCT. In some embodiments, the composition further contains squalane. The composition may further contain one or more carriers, diluents, or excipients.

In another aspect, the disclosure features a cosmetic composition containing CBG and CCT, optionally in combination with one or more carriers, diluents, or excipients. The composition may further contain squalane.

In another aspect, the disclosure features a cosmetic composition containing CBG and squalane, optionally in combination with one or more carriers, diluents, or excipients. The composition may further contain CCT.

In some embodiments of any of the foregoing aspects of the disclosure, the cosmetic composition has a final concentration of from about 0.1% by weight (w/w) to about 10% w/w CBG (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% CBG). In some embodiments of any of the foregoing aspects of the disclosure, the cosmetic composition has a final concentration of from about 0.5% w/w to about 5% w/w CBG (e.g., 0.5%, 0.6%, 0.7%, 0.8%, 0.9%, 1%, 1.5%, 2%, 2%, 2.5%, 3%, 3.5%, 4%, 4.5%, or 5% CBG. In some embodiments, the cosmetic composition has a final concentration of about 1% w/w CBG. In some embodiments, the cosmetic composition has a final concentration of about 2% w/w CBG. In some embodiments, the cosmetic composition has a final concentration of about 4% w/w CBG.

In some embodiments of any of the foregoing aspects of the disclosure, the cosmetic composition further includes an additional cannabinoid. In some embodiments, the additional cannabinoid is CBD. In some embodiments, the composition further includes one or more essential oils. For example, the composition may further include 1, 2, 3, 4, 5, or more, essential oils. In some embodiments, the one or more essential oils include Lavandula angustifolia oil. In some embodiments, the one or more essential oils include hemp oil. The hemp oil may be, for example, Cannabis sativa seed oil. In some embodiments, the one or more essential oils include Lavandula angustifolia oil and Cannabis sativa seed oil.

In some embodiments of any of the foregoing aspects or embodiments of the disclosure, the cosmetic composition further includes one or more plant extracts. In some embodiments, the plant extract is a flower, leaf, or stem extract. In some embodiments, the plant extract is a Cannabis sativa flower, leaf, or stem extract.

In some embodiments of any of the foregoing aspects or embodiments of the disclosure, the cosmetic composition is formulated for topical administration to the skin of a human subject (e.g., a male or female human subject). In some embodiments, the composition is a patch, liquid, gel, lotion, paste, cream, foam, serum, ointment, or stick.

In a further aspect, the disclosure features a method of improving the outward appearance of the skin of a human subject (e.g., a male or female human subject) by administering to the subject the cosmetic composition of any of the above aspects or embodiments of the disclosure.

In another aspect, the disclosure features a method of improving skin barrier function in a human subject (e.g., a male or female human subject) by administering to the subject the cosmetic composition of any of the above aspects or embodiments of the disclosure.

In another aspect, the disclosure features a method of reducing transepidermal water loss in a human subject (e.g., a male or female human subject) by administering to the subject the cosmetic composition of any of the above aspects or embodiments of the disclosure.

In another aspect, the disclosure features a method of reducing redness in the skin of a human subject (e.g., a male or female human subject) by administering to the subject the cosmetic composition of any of the above aspects or embodiments of the disclosure.

In another aspect, the disclosure features a method of reducing erythema, induration, flaking, and/or scaling in the skin of a human subject (e.g., a male or female human subject) by administering to the subject the cosmetic composition of any of the above aspects or embodiments of the disclosure. In some embodiments, the erythema, induration, flaking, and/or scaling is due to psoriasis.

In another aspect, the disclosure features a method of reducing acne in a human subject (e.g., a male or female human subject) by administering to the subject the cosmetic composition of any of the above aspects or embodiments of the disclosure.

In another aspect, the disclosure features a method of brightening the skin of a human subject (e.g., a male or female human subject) by administering to the subject the cosmetic composition of any of the above aspects or embodiments of the disclosure.

In another aspect, the disclosure features a method of increasing the luminosity of the skin of a human subject (e.g., a male or female human subject) by administering to the subject the cosmetic composition of any of the above aspects or embodiments of the disclosure.

In another aspect, the disclosure features a method of increasing skin tone uniformity in a human subject (e.g., a male or female human subject) by administering to the subject the cosmetic composition of any of the above aspects or embodiments of the disclosure.

In another aspect, the disclosure features a method of reducing dullness in the skin of a human subject (e.g., a male or female human subject) by administering to the subject the cosmetic composition of any of the above aspects or embodiments of the disclosure.

In another aspect, the disclosure features a method of reducing discoloration in the skin of a human subject (e.g., a male or female human subject) by administering to the subject the cosmetic composition of any of the above aspects or embodiments of the disclosure.

In some embodiments, the cosmetic composition increases skin hydration, smooths skin, renews skin, and/or cleanses skin. In some embodiments, the composition reduces skin inflammation. In some embodiments, the skin inflammation is caused by dermatitis, psoriasis, rosacea, or exposure to ultraviolet (UV) radiation. In some embodiments, the skin inflammation is contact dermatitis, seborrheic dermatitis, nummular dermatitis, stasis dermatitis, atopic dermatitis, and dermatitis herpetiformis, among others In some embodiments, the skin inflammation is chronic. In some embodiments, the skin inflammation is acute.

In another aspect, the disclosure features a method of treating or preventing inflammation (e.g., chronic inflammation or acute inflammation) in a subject (e.g., a male or female human subject) by administering to the subject a pharmaceutical composition containing CBG. The composition may further contain CCT and/or squalane. In some embodiments, the composition contains one or more (e.g., 1, 2, 3, 4, 5, or more) additional cannabinoids. For example, the additional cannabinoid may be CBD. In some embodiments, the composition contains one or more (e.g., 1, 2, 3, 4, 5, or more) essential oils. The one or more essential oils may include, for example, Lavandula angustifolia oil and/or hemp oil. The hemp oil may be, for example, Cannabis sativa oil. In some embodiments, the composition contains one or more (e.g., 1, 2, 3, 4, 5, or more) plant extracts. For example, the plant extract may be a flower, leaf, or stem extract. In some embodiments, the flower, leaf, or stem extract is Cannabis sativa flower, leaf, or stem extract.

In another aspect, the disclosure features a method of treating or preventing acne in a subject (e.g., a male or female human subject) by administering to the subject a pharmaceutical composition containing CBG. The composition may further contain CCT and/or squalane. In some embodiments, the composition contains one or more (e.g., 1, 2, 3, 4, 5, or more) additional cannabinoids. For example, the additional cannabinoid may be CBD. In some embodiments, the composition contains one or more (e.g., 1, 2, 3, 4, 5, or more) essential oils. The one or more essential oils may include, for example, Lavandula angustifolia oil and/or hemp oil. The hemp oil may be, for example, Cannabis sativa oil. In some embodiments, the composition contains one or more (e.g., 1, 2, 3, 4, 5, or more) plant extracts. For example, the plant extract may be a flower, leaf, or stem extract. In some embodiments, the flower, leaf, or stem extract is Cannabis sativa flower, leaf, or stem extract.

In another aspect, the disclosure features a method of treating or preventing psoriasis in a subject (e.g., a male or female human subject) by administering to the subject a pharmaceutical composition containing CBG. The composition may further contain CCT and/or squalane. In some embodiments, the composition contains one or more (e.g., 1, 2, 3, 4, 5, or more) additional cannabinoids. For example, the additional cannabinoid may be CBD. In some embodiments, the composition contains one or more (e.g., 1, 2, 3, 4, 5, or more) essential oils. The one or more essential oils may include, for example, Lavandula angustifolia oil and/or hemp oil. The hemp oil may be, for example, Cannabis sativa oil. In some embodiments, the composition contains one or more (e.g., 1, 2, 3, 4, 5, or more) plant extracts. For example, the plant extract may be a flower, leaf, or stem extract. In some embodiments, the flower, leaf, or stem extract is Cannabis sativa flower, leaf, or stem extract.

In another aspect, the disclosure features a method of treating or preventing eczema in a subject (e.g., a male or female human subject) by administering to the subject a pharmaceutical composition containing CBG. The composition may further contain CCT and/or squalane. In some embodiments, the composition contains one or more (e.g., 1, 2, 3, 4, 5, or more) additional cannabinoids. For example, the additional cannabinoid may be CBD. In some embodiments, the composition contains one or more (e.g., 1, 2, 3, 4, 5, or more) essential oils. The one or more essential oils may include, for example, Lavandula angustifolia oil and/or hemp oil. The hemp oil may be, for example, Cannabis sativa oil. In some embodiments, the composition contains one or more (e.g., 1, 2, 3, 4, 5, or more) plant extracts. For example, the plant extract may be a flower, leaf, or stem extract. In some embodiments, the flower, leaf, or stem extract is Cannabis sativa flower, leaf, or stem extract.

In another aspect, the disclosure features a method of treating or preventing rosacea in a subject (e.g., a male or female human subject) by administering to the subject a pharmaceutical composition containing CBG. The composition may further contain CCT and/or squalane. In some embodiments, the composition contains one or more (e.g., 1, 2, 3, 4, 5, or more) additional cannabinoids. For example, the additional cannabinoid may be CBD. In some embodiments, the composition contains one or more (e.g., 1, 2, 3, 4, 5, or more) essential oils. The one or more essential oils may include, for example, Lavandula angustifolia oil and/or hemp oil. The hemp oil may be, for example, Cannabis sativa oil. In some embodiments, the composition contains one or more (e.g., 1, 2, 3, 4, 5, or more) plant extracts. For example, the plant extract may be a flower, leaf, or stem extract. In some embodiments, the flower, leaf, or stem extract is Cannabis sativa flower, leaf, or stem extract.

In another aspect, the disclosure features a method of treating or preventing UV radiation-induced damage to the skin of a subject (e.g., a male or female human subject) by administering to the subject a pharmaceutical composition containing CBG. The composition may further contain CCT and/or squalane. In some embodiments, the composition contains one or more (e.g., 1, 2, 3, 4, 5, or more) additional cannabinoids. For example, the additional cannabinoid may be CBD. In some embodiments, the composition contains one or more (e.g., 1, 2, 3, 4, 5, or more) essential oils. The one or more essential oils may include, for example, Lavandula angustifolia oil and/or hemp oil. The hemp oil may be, for example, Cannabis sativa oil. In some embodiments, the composition contains one or more (e.g., 1, 2, 3, 4, 5, or more) plant extracts. For example, the plant extract may be a flower, leaf, or stem extract. In some embodiments, the flower, leaf, or stem extract is Cannabis sativa flower, leaf, or stem extract.

In another aspect, the disclosure features a method of treating or preventing dermatitis in a subject (e.g., a male or female human subject) by administering to the subject a pharmaceutical composition containing CBG. The composition may further contain CCT and/or squalane. In some embodiments, the composition contains one or more (e.g., 1, 2, 3, 4, 5, or more) additional cannabinoids. For example, the additional cannabinoid may be CBD. In some embodiments, the composition contains one or more (e.g., 1, 2, 3, 4, 5, or more) essential oils. The one or more essential oils may include, for example, Lavandula angustifolia oil and/or hemp oil. The hemp oil may be, for example, Cannabis sativa oil. In some embodiments, the composition contains one or more (e.g., 1, 2, 3, 4, 5, or more) plant extracts. For example, the plant extract may be a flower, leaf, or stem extract. In some embodiments, the flower, leaf, or stem extract is Cannabis sativa flower, leaf, or stem extract.

For example, in some embodiments, the disclosure features a method of treating or preventing contact dermatitis, seborrheic dermatitis, nummular dermatitis, stasis dermatitis, atopic dermatitis, and/or dermatitis herpetiformis in a subject (e.g., a male or female human subject) by administering to the subject a pharmaceutical composition containing CBG. The composition may further contain CCT and/or squalane. In some embodiments, the composition contains one or more (e.g., 1, 2, 3, 4, 5, or more) additional cannabinoids. For example, the additional cannabinoid may be CBD. In some embodiments, the composition contains one or more (e.g., 1, 2, 3, 4, 5, or more) essential oils. The one or more essential oils may include, for example, Lavandula angustifolia oil and/or hemp oil. The hemp oil may be, for example, Cannabis sativa oil. In some embodiments, the composition contains one or more (e.g., 1, 2, 3, 4, 5, or more) plant extracts. For example, the plant extract may be a flower, leaf, or stem extract. In some embodiments, the flower, leaf, or stem extract is Cannabis sativa flower, leaf, or stem extract.

In some embodiments of any of the foregoing aspects or embodiments of the disclosure, the cosmetic composition or pharmaceutical composition is administered to the subject topically. In some embodiments, the composition is administered to the subject one or more times (e.g., 2 times, 3 times, 4 times, 5 times, or more) daily. In some embodiments, the composition is administered to the subject once daily. In some embodiments, the composition is administered two times a day. In some embodiments, the composition is administered three times a day. In some embodiments, the composition is administered twice in the morning and once at night. In some embodiments, the composition is administered to the subject for two or more continuous days. For example, the composition may be administered to the subject over the course of a period having a duration of from 2 continuous days to 30 continuous days, or more, such as a period having a duration of 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more. In some embodiments, the composition is administered to the subject for at least 7 continuous days (e.g., for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more). In some embodiments, the composition is administered to the subject for at least 14 continuous days (e.g., for 14 days, 15 days, 16 days, 17 days, 18 days, 19 days, 20 days, 21 days, 22 days, 23 days, 24 days, 25 days, 26 days, 27 days, 28 days, 29 days, 30 days, or more). In some embodiments, the composition is administered to the subject for at least 21 continuous days (e.g., for 21 days, 22 days, 23 days, 24 days, 25 days, 26 days, 27 days, 28 days, 29 days, 30 days, or more). In some embodiments, the composition is administered to the subject for at least 28 continuous days (e.g., for 21 days, 22 days, 23 days, 24 days, 25 days, 26 days, 27 days, 28 days, 29 days, 30 days, or more).

In another aspect, the disclosure features a method of manufacturing the cosmetic composition or pharmaceutical composition of any of the above aspects or embodiment of the disclosure. In some embodiments, the method includes admixing CBG in one or more carriers, diluents, or excipients. In some embodiments, the CBG is further admixed with CCT. In some embodiments, the CBG is further admixed with squalane. In some embodiments, the CBG is further admixed with an additional cannabinoid, such as CBD.

In some embodiments, the CBG is further admixed with one or more (e.g., 2, 3, 4, 5, or more) essential oils. In some embodiments, the one or more essential oils include Lavandula angustifolia oil. In some embodiments, the one or more essential oils include hemp oil. The hemp oil may be, for example, Cannabis sativa seed oil. In some embodiments, the one or more essential oils include Lavandula angustifolia oil and Cannabis sativa seed oil.

In some embodiments, the CBG is further admixed with one or more (e.g., 2, 3, 4, 5, or more) plant extracts. In some embodiments, the plant extract is a flower, leaf, or stem extract. In some embodiments, the plant extract is a flower, leaf, or stem extract.

In some embodiments, the cosmetic composition has a final concentration of from about 0.1% w/w to about 10% w/w CBG (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% CBG). In some embodiments, the cosmetic composition has a final concentration of from about 0.5% w/w to about 5% w/w CBG (e.g., 0.5%, 0.6%, 0.7%, 0.8%, 0.9%, 1%, 1.5%, 2%, 2%, 2.5%, 3%, 3.5%, 4%, 4.5%, and 5% CBG. In some embodiments, the cosmetic composition has a final concentration of about 1% w/w CBG. In some embodiments, the cosmetic composition has a final concentration of about 2% w/w CBG. In some embodiments, the cosmetic composition has a final concentration of about 4% w/w CBG.

In some embodiments, the cosmetic composition or pharmaceutical composition is formulated for topical administration to the skin of a human subject (e.g., a male or female human subject). In some embodiments, the composition is a patch, liquid, gel, lotion, paste, cream, foam, serum, ointment, or stick.

### BRIEF DESCRIPTION OF THE DRAWINGS

FIG. 1 is a graph illustrating the effects of various cosmetic compositions containing cannabigerol (CBG) on transepidermal water loss (TEWL), an indicator of skin barrier function. The compositions that were tested included Test Articles 1-3 described in Example 1, below. TEWL measurements were recorded on Day 1 before treatment, on Day 1 after 10 minutes of treatment, on Day 4, on Day 9, and on Day 15, in accordance with the protocol outlined in Example 1.

FIG. 2 is a graph illustrating the effects of various cosmetic compositions containing CBG on skin irritancy. The compositions that were tested included Test Articles 1-3 described in Example 1, below. Irritancy score measurements were recorded on Day 1 before treatment, on Day 1 after 10 minutes of treatment, on Day 2, on Day 3, on Day 4, on Day 7, on Day 8, on Day 9, on Day 10, on Day 11, and on Day 14, in accordance with the protocol outlined in Example 1.

FIG. 3 is a series of photographic images showing the effect of a cosmetic composition containing CBG on a psoriasis lesion. The images were obtained prior to and following administration of Test Article 3, as is described in Example 4, below. The images were obtained at baseline and at Week 1, Week 2, and Week 4 during the clinical trial period described in Example 4.

FIG. 4 is a series of photographic images showing the effect of a cosmetic composition containing CBG on a psoriasis lesion. The images were obtained prior to and following administration of Test Article 3, as is described in Example 4, below. The images were obtained at baseline and at Week 1, Week 2, and Week 4 during the clinical trial period described in Example 4.

FIG. 5 is a series of photographic images showing the effect of a cosmetic composition containing CBG on the outward appearance of the skin of a human subject having acne. The images were obtained prior to and following administration of Test Article 3, as is described in Example 5, below. The images were obtained at baseline and at Week 1, Week 2, and Week 4 during the clinical trial period described in Example 5.

FIGS. 6A-6E are a series of photographic images showing the effect of a cosmetic composition containing CBG on the skin of a human subject having an eczema lesion. The images were obtained prior to (FIG. 6A) and following (FIGS. 6B-6E) administration of Test Article 3, as is described in Example 6, below.

FIG. 7 is a graph showing the results of a self-perception questionnaire obtained from subjects treated with Test Article 2 in accordance with the study described in Example 5, below. The results of the questionnaire demonstrate that Test Article 2 was capable of brightening subjects' skin, improving subjects' overall skin tone, and generally improving the outward appearance of subjects' skin.

FIG. 8 is a graph showing the results of a self-perception questionnaire obtained from subjects treated with Test Article 3 in accordance with the study described in Example 5, below. The results of the questionnaire demonstrate that Test Article 3 was capable of brightening subjects' skin, improving subjects' overall skin tone, and generally improving the outward appearance of subjects' skin.

### DEFINITIONS

As used herein the singular forms “a,” “an,” and, “the” include plural reference unless the context clearly dictates otherwise.

The term “about” when modifying a numerical value or range herein includes normal variation encountered in the field, and specifically includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the terms “administering” and “administration” refer to the administration of a composition (e.g., a compound or a preparation that includes a compound as described herein) to a subject. Administration to a subject may be by any appropriate route; for example, the administration may be topical administration, for instance, in the form of a patch, liquid, gel, lotion, paste, cream, foam, serum, ointment, or stick.

As used herein in the context of improving the outward appearance of a subject's skin, the term “brighten” refers to a process of increasing a subject's skin tone uniformity, skin complexion uniformity, skin luminosity, and/or skin firmness. The term “brighten” may also refer to a process of decreasing discoloration and/or dullness in a subject's skin, which may be caused by an accumulation of dead cells on the skin surface. Each of the foregoing parameters—skin tone uniformity, skin complexion uniformity, skin luminosity, skin firmness, skin discoloration, and skin dullness—can be evaluated by a visual inspection of a subject's skin. For example, an increase in skin tone uniformity can be readily detected by visually monitoring a subject's skin and assessing whether the subject's skin tone at a location of interest has become more evenly distributed, with fewer areas of dissimilar color. Similarly, skin complexion uniformity can be evaluated by visually monitoring a subject's skin and assessing whether the texture of the subject's skin has become increasingly even, with fewer fluctuations in texture from one area to another. Skin luminosity can also be assessed by visual evaluation of the intensity of light that is reflected from the surface of a subject's skin. Additionally or alternatively, skin luminosity can be evaluated using photodetection methods known in the art for assessing the intensity of reflected light, for example, using methods described in Jeudy et al. (2015) Measurement of Skin Radiance. In: Humbert et al., Agache's Measuring the Skin. ISBN: 978-3-319-26594-0, the disclosure of which is incorporated herein by reference.

As used herein, the term “cannabinoid” refers to a chemical substance that binds or interacts with a cannabinoid receptor (for example, a human cannabinoid receptor) and includes, without limitation, chemical compounds such endocannabinoids, phytocannabinoids, and synthetic cannabinoids. Synthetic compounds are chemicals made to mimic phytocannabinoids which are naturally found in the cannabis plant (e.g., Cannabis sativa), including but not limited to cannabigerols (CBG), cannabichromens (CBC), cannabidiol (CBD), tetrahydrocannabinol (THC), cannabinol (CBN), cannabinodiol (CBDL), cannabicyclol (CBL), cannabielsoin (CBE), and cannabitriol (CBT).

As used herein, the term “cannabidiol” or “CBD” refers to one of the cannabinoid compounds found in the Cannabis plant having the following structure:

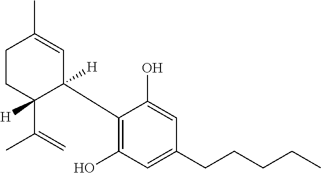 [SMILES: C=C(C)[C@@H]1CCC(C)=C[C@H]1c1c(O)cc(CCCCC)cc1O]

As used herein, the term “cannabigerol” or “CBG” refers to one of the cannabinoid compounds found in the Cannabis plant having the following structure:

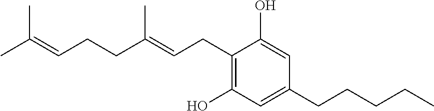 [SMILES: CCCCCc1cc(O)c(C/C=C(\C)CCC=C(C)C)c(O)c1]

As used herein, the term “Cannabis sativa seed oil” refers to a hemp essential oil distilled from the seeds of the Cannabis sativa plant. The physical and chemical properties of Cannabis sativa seed oil are described, e.g., in Oomah et al., Food Chemistry 76:33-43 (2002), the disclosure of which is incorporated herein by reference in its entirety.

As used herein, the term “Cannabis sativa flower, leaf, or stem extract” refers to a plant extract from the flower, leaf, or stem of the Cannabis sativa plant. The components of Cannabis sativa extract are described, for example, in Pertwee, Roger, editor. Handbook of Cannabis. Oxford University Press, 2016, the disclosure of which is incorporated herein by reference.

As used herein, the term “capric/caprylic triglyceride” and its abbreviation, “CCT,” refers to a mixture of esters including caprylic and capric fatty acids covalently bound to a glycerin backbone. Capric/caprylic triglyceride may include from about 50% to about 70% caprylic acid and from about 30% to about 50% caprid acid. Using International Union of Pure and Applied Chemistry (IUPAC) nomenclature, capric/caprylic triglyceride is also referred to using as decanoic acid esterified with 1,2,3-propanetriol octanoate.

As used herein, the term “cosmetic composition” refers to a composition that is intended to be applied to a user's skin (e.g., the skin of a male or female human subject) so as to regulate a condition of the skin and/or to improve the outward appearance of the skin. Cosmetic compositions of the disclosure may further include one or more carriers, diluents, or excipients, such as a carrier, diluent, or excipient described herein.

As used herein, the terms “decrease” and “reduce” refer to reduction in the level of a property of interest by a statistically significant or visually apparent amount as compared to a reference level of the property. The reference level may be, for example, a level observed in the absence of using a cosmetic composition of the disclosure. In some embodiments of the disclosure, the “decrease” or “reduction” observed in connection with a particular property (such as inflammation, redness, or dehydration, among other epidermal properties described herein) is, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. A “decrease” or “reduction” may also refer to a reduction in a particular property that is readily apparent from a visual inspection of a subject. For example, a “decrease” or “reduction” in skin redness refers to a reduction in skin redness that can be readily observed by the user of a cosmetic composition of the disclosure upon visual inspection of the affected area of the skin. A “decrease” or “reduction” in visually assessed properties, such as skin redness, may be observed by examining photographic images of a subject before and after administration of a cosmetic composition of the disclosure to the affected area of the skin.

As used herein, the term “essential oil” refers to a concentrated hydrophobic liquid containing one or more volatile organic compounds produced by a plant, such as Cannabis sativa or another plant described herein. Essential oils are also referred to as volatile oils, ethereal oils, or aetherolea.

As used herein, the terms “increase,” “enhance,” and “improve” refer to an increase in a property of interest by a statistically significant or visually apparent amount as compared to a reference level of the property. The reference level may be, for example, a level observed in the absence of using a cosmetic composition of the disclosure. In some embodiments of the disclosure, the “increase,” “improvement,” or “enhancement” observed in connection with a particular property (such as skin barrier function or skin hydration, among other beneficial epidermal properties described herein) is, for example, an increase by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. An “increase,” “improvement,” or “enhancement” may also refer to an improvement in a particular property that is readily apparent from a visual inspection of a subject. For example, an “increase,” “improvement,” or “enhancement” in skin hydration refers to an increase in skin hydration that can be readily observed by the user of a cosmetic composition of the disclosure upon visual inspection of the affected area of the skin. An “increase,” “improvement,” or “enhancement” in visually assessable properties, such as skin hydration, may be observed by examining photographic images of a subject before and after administration of a cosmetic composition of the disclosure to the affected area of the skin.

As used herein, the term “Lavandula angustifolia oil” refers to lavender essential oil distilled from the Lavandula angustifolia plant. The physical and chemical properties of Lavandula angustifolia oil are described, for example, in Reza Fakhari et al., Journal of Chromatography A, 1098:14-18 (2005), the disclosure of which is incorporated herein by reference in its entirety.

As used herein, the term “squalane” refers to linear hydrocarbon obtainable from squalene by way of a hydrogenation reaction. Squalane has the following chemical structural formula:

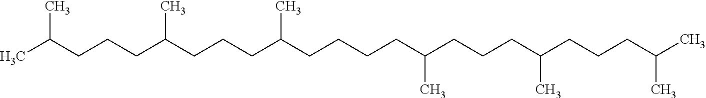 [SMILES: CC(C)CCCC(C)CCCC(C)CCCCC(C)CCCC(C)CCCC(C)C]

As used herein, the term “skin barrier function” refers to the ability of the skin of a subject (e.g., a male or female human subject) to retain hydration and prevent excessive transepidermal water loss. Skin barrier function can be assessed, for example, by monitoring transepidermal water loss in a subject using a transepidermal water loss assay described herein, such as the transepidermal water loss assay outlined in Example 1, below.

As used herein, the term “subject” refers to an animal, such as a mammal (e.g., a male or female human), to which a cosmetic composition described herein may be administered.

As used herein in the context of administration of a cosmetic composition to a subject, the term “topical” refers to administration of the cosmetic composition to any skin or exposed mucosal surface. Skin surface includes any exposed epidermal region of the subject's body, including, without limitation, the skin of the subject's face, hands, legs, neck, abdominal area, eyes, nose, and chest.

As used herein, the term “transepidermal water loss” refers to the amount of water that is lost to evaporation over the skin barrier provided by the epidermis. A variety of methods may be used to measure transepidermal water loss. Examples of such methods are provided in, e.g., Antonov et al., Curr. Probl. Dermatol. 49:61-70 (2016), the disclosure of which is incorporated herein by reference.

As used herein, “treatment” and “treating” refer to an approach for obtaining beneficial or desired results, e.g., therapeutic results. Beneficial or desired results can include, but are not limited to, alleviation or amelioration of one or more symptoms or conditions; diminishment of extent of disease or condition; stabilized (i.e., not worsening) state of disease or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease or condition are lessened and/or time course of the progression is slowed or lengthened, as compared to the extent or time course in the absence of treatment. Those in need of treatment include those already with the disease or condition, those prone to or at risk of developing the disease or condition, and those in which the disease or condition is to be prevented.

### DETAILED DESCRIPTION OF THE INVENTION

The present disclosure provides compositions (e.g., cosmetic compositions) that contain cannabigerol (CBG), optionally in combination with one or more carriers, diluents, or excipients. The compositions (e.g., cosmetic compositions) of the disclosure may further include capric/caprylic triglycerides (CCT). In some embodiments, the compositions (e.g., cosmetic compositions) of the disclosure may include squalane. The compositions (e.g., cosmetic compositions) may also have one or more additional components, such as an additional cannabinoid, one or more essential oils, and/or one or more plant extracts. Additionally, the disclosure provides methods of using the compositions (e.g., cosmetic compositions) described herein to improve the outward appearance of the skin of a subject (e.g., a male or female human subject), as well as methods of using the compositions (e.g., cosmetic compositions) to improve skin barrier functionality in a subject (e.g., a male or female human subject).

The present disclosure is based, in part, on the discovery that compositions containing CBG can reduce transepidermal water loss, thereby improving hydration of a subject's skin. It has also presently been discovered that compositions containing CBG can reduce redness and other blemishes in a subject's skin, such as redness and other imperfections arising from chronic or acute inflammation. The compositions of the disclosure, as well as methods of using the same to achieve these beneficial cosmetic outcomes, are described in further detail below.

### Components of Compositions of the Disclosure

### Cannabinoids

In some embodiments, the composition (e.g., cosmetic composition) includes a cannabinoid. Examples of suitable cannabinoids that may be used in conjunction with the compositions and methods of the disclosure include those that are naturally found in the cannabis plant (e.g., Cannabis sativa), including, without limitation, CBG, cannabichromens (CBC), cannabidiol (CBD), tetrahydrocannabinol (THC), cannabinol (CBN), cannabinodiol (CBDL), cannabicyclol (CBL), cannabielsoin (CBE), and cannabitriol (CBT). In some embodiments, the composition (e.g., cosmetic composition) includes CBG. In some embodiments, the composition (e.g., cosmetic composition) includes CBG, which has the following chemical structure:

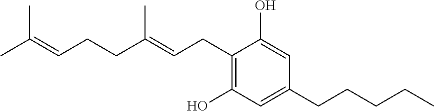 [SMILES: CCCCCc1cc(O)c(C/C=C(\C)CCC=C(C)C)c(O)c1]

In some embodiments, the composition (e.g., cosmetic composition) includes CBD, which has the following chemical structure:

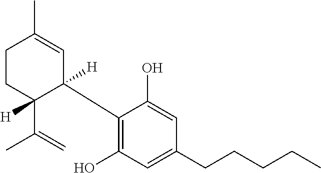 [SMILES: C=C(C)[C@@H]1CCC(C)=C[C@H]1c1c(O)cc(CCCCC)cc1O]

In some embodiments, the composition (e.g., cosmetic composition) includes one or more (e.g., 2, 3, 4, 5, or more) cannabinoids. For example, in some embodiments, the composition (e.g., cosmetic composition) includes both CBG and CBD.

Natural Sources of Cannabinoids

The cannabinoid used in any of the compositions of the disclosure may be obtained from any suitable source. For example, in some embodiments, the cannabinoid may be isolated from hemp oil; for example, the cannabinoid may be from Cannabis sativa seed oil. In some embodiments, the cannabinoid may be from plant extract (e.g., hemp extract); for example, the cannabinoid may be from a Cannabis sativa flower, leaf, or stem extract. In some embodiments, the cannabinoid may be produced exogenously.

Heterologous Expression of Cannabinoids from Host Cells

In some embodiments, the cannabinoid may be produced using a host cell (e.g., a yeast cell) that is modified to express one or more enzymes of the cannabinoid biosynthetic pathway and is thus capable of producing a cannabinoid or a precursor of a cannabinoid. In some embodiments, the precursor is a substrate in the cannabinoid pathway. In some embodiments, the precursor is a substrate for hexanoyl-CoA synthase (HCS), tetraketide synthase (TKS), or olivetolic acid cyclase (OAC). In some embodiments, the precursor, substrate or intermediate in the cannabinoid pathway is hexanoate, olivetol, or olivetolic acid. In some embodiments, the precursor is hexanoate. In some embodiments, the host cell does not comprise the precursor, substrate or intermediate in an amount sufficient to produce the cannabinoid or a precursor of the cannabinoid. In some embodiments, the host cell does not comprise hexanoate at a level or in an amount sufficient to produce the cannabinoid in an amount over 10 mg/L. In some embodiments, the heterologous genetic pathway encodes at least two enzymes selected from the group consisting of hexanoyl-CoA synthase (HCS), tetraketide synthase (TKS) and olivetolic acid cyclase (OAC).

The cannabinoid biosynthetic pathway is described in Keasling et al., WO 2018/200888, and methods of producing cannabinoids heterologously in yeast cells are described in Klein et al., WO 2020/190763, the disclosures of each of which are incorporated herein by reference.

In some embodiments, host cell (e.g., yeast cell) expresses one or more genes of the cannabinoid biosynthesis pathway under the control of a genetic regulatory element that is induced or repressed by an exogenous agent. In some embodiments, the exogenous agent can be used as a carbon source by the host cell. For example, the same exogenous agent may both regulate expression of the cannabinoid biosynthetic pathway and provide a carbon source for growth of the host cell. In some embodiments, the exogenous agent is glucose. In some embodiments, the exogenous agent is galactose. In some embodiments, the exogenous agent is maltose.

In some embodiments, the genetic regulatory element is a nucleic acid sequence, such as a promoter. In some embodiments, the genetic regulatory element is a glucose-responsive promoter or a promoter that is repressed by glucose. In some embodiments, glucose negatively regulates expression of the heterologous genetic pathway, thereby decreasing production of the cannabinoid. Exemplary glucose repressed promoters include pMAL11, pMAL12, pMAL13, pMAL21, pMAL22, pMAL31, pMAL32, pMAL33, pCAT8, pHXT2, pHXT4, pMTHI, and pSUC2, which are described in WO 2020/190763.

In some embodiments, the genetic regulatory element is a galactose-responsive promoter. In some embodiments, galactose positively regulates expression of the cannabinoid biosynthetic pathway, thereby increasing production of the cannabinoid product. In some embodiments, the galactose-responsive promoter is a GAL1 promoter. In some embodiments, the galactose-responsive promoter is a GAL10 promoter. In some embodiments, the galactose-responsive promoter is a GAL2, GAL3, or GAL7 promoter. In some embodiments, heterologous genetic pathway comprises the galactose-responsive regulatory elements described in Westfall et al., PNAS 109:E111-118 (2012). In some embodiments, the host cell lacks the gal gene and is unable to metabolize galactose, but galactose can still induce galactose-regulated genes.

In some embodiments, the galactose regulation system used to control expression of heterologous genes is re-configured such that it is no longer induced by the presence of galactose. Instead, the genes will be expressed unless repressors, which may be lysine in some strains or maltose in other strains, are present in the media.

In some embodiments, the genetic regulatory element is a maltose-responsive promoter. In some embodiments, maltose negatively regulates expression of the heterologous genetic pathway, thereby increasing production of the heterologous product. In some embodiments, the maltose maltose-responsive promoter is selected from the group consisting of pMALI, pMAL2, pMALII, pMAL12, pMAL31 and pMAL32. The maltose genetic regulatory element can be designed to both activate expression of some genes and repress expression of others, depending on whether maltose is present or absent in the medium. Maltose regulation of gene expression and maltose-responsive promoters are described in U.S. Patent Publication 2016/0177341, which is hereby incorporated by reference. Genetic regulation of maltose metabolism is described in Novak et al., Food Technol. Biotechnol. 42:213-218 (2004).

In some embodiments, the heterologous cannabinoid biosynthetic pathway is regulated by a combination of the maltose and galactose regulons.

In some embodiments, the heterologous cannabinoid pathway is regulated by lysine. The regulation of LYS genes is described, for example, by Feller et al., Eur. J. Biochem. 261:163-170 (1999).

Exemplary host cells that can be used to express one or more enzymes of the cannabinoid biosynthetic pathway include yeast. Yeast strains that may be used to heterologously produce a cannabinoid for use in conjunction with the compositions and methods of the disclosure include, without limitation, yeast belonging to the Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, lssatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Satumospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the yeast strain used to produce a cannabinoid described herein is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host cell is a yeast strain of the genus Candida, such as Candida lipolytica, Candida guilliermondii, Candida krusei, Candida pseudotropicalis, or Candida utilis.

In some embodiments, the host cell is Saccharomyces cerevisiae. In some embodiments, the host cell is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is selected from the group consisting of PE-2, CAT-1, VR-1, BG-1, CR-1, and SA-1.

In some embodiments, the yeast strain is one that is suitable for industrial fermentation. In some embodiments, the yeast strain is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

In some embodiments, the yeast strain is a Y27598, Y27599, Y27600, Y27601 Y27602, Y27603, Y27604 or Y25618 strain. Additional examples of yeast strains that may be used to produce a cannabinoid of the disclosure are described, e.g., in WO 2020/190763.

### Capric/Caprylic Triglyceride

In some embodiments, the composition (e.g., cosmetic composition) includes capric/caprylic triglyceride. Capric/caprylic triglyceride is a mixture of esters including of the eight carbon containing caprylic acid and the ten carbon containing capric acid covalently bound to a glycerin backbone. Capric/Caprylic triglyceride includes between 50% and 70% caprylic acid and 30% to 50% capric acid.

Capric/caprylic triglyceride may be isolated from coconut oil or palm kernel oil. Capric/caprylic triglyceride may purified by separating the capric acid and the caprylic acid from the glycerol backbone by way of a saponification reaction or steam hydrolysis. The capric acid, caprylic acid, and glycerol may then undergo an esterification reaction to generate pure capric/caprylic triglyceride. In some embodiments, capric/caprylic acid may be exogenously synthesized, for example, as described in WO 2013/126990, the disclosure of which is incorporated herein by reference.

### Squalane

In some embodiments, the composition (e.g., cosmetic composition) includes squalane. Squalane is a linear hydrocarbon having the following chemical structure:

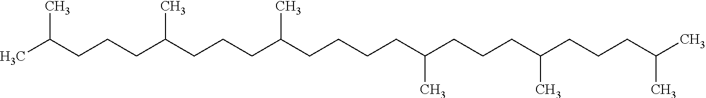 [SMILES: CC(C)CCCC(C)CCCC(C)CCCCC(C)CCCC(C)CCCC(C)C]

Squalane is the complete saturated product of hydrogenated squalene.

Squalane may be derived naturally from the livers of sharks, olive oil, rice, and sugar cane. In some embodiments, squalane may be chemically synthesized. For example, squalane may be synthesized from the dimerization reaction of farnesene to isosqualene, followed by a hydrogenation reaction as described in EP 2574187, the disclosure of which is incorporated herein by reference.

### Essential Oils

In some embodiments, the composition (e.g., cosmetic composition) includes an essential oil. Essential oils are mixtures of various organic compounds isolated from a plan, and may include, for example, one or more terpenes, alcohols, esters, aldehydes, ketones, and/or phenols. Synthetic oils may be produced from one or more of the constituents predominant within a particular essential oil; menthol, for example, often substitutes for mint and eucalyptol for eucalyptus. Essential oils come from various species of flowers, grasses, fruits, leaves and trees. They are found in the bark, seeds, leaves, petals, stems, and roots of plants. Essential oils may be extracted from the plant using a number of methods including but not limited to steam distillation, cold expression, solvent extraction, and carbon dioxide extraction.

In some embodiments, the composition (e.g., cosmetic composition) includes one or more (e.g., 2, 3, 4, 5, or more) essential oils. The one or more essential oils may include, for example, hemp oil, lavender oil, clary sage oil, cypress oil, eucalyptus oil, fennel oil, geranium oil, ginger oil, helichrysum oil, lemon oil, lemongrass oil, mandarin oil, neroli oil, patchouli oil, peppermint oil, Roman chamomile oil, rose oil, rosemary oil, tea tree oil, vetiver oil, ylang oil, among other essential oils. In some embodiments, the cosmetic composition includes the essential oil hemp oil. In some embodiments, the hemp oil is Cannabis sativa seed oil. In some embodiments, the essential oil is Lavandula angustifolia oil.

### Plant Extracts

In some embodiments, the composition (e.g., cosmetic composition) includes a plant extract. Examples of plant extracts suitable for use in conjunction with the compositions and methods of the disclosure include, without limitation, Cannabis sativa flower, leaf, or stem extract, Harpagophytum procumbens extract, Hedera helix extract, Pelargonium sidoides extract, Zingiber officinale extract, gingko extract, mistletoe extract, and Sativex extract, among others. In some embodiments, the composition (e.g., cosmetic composition) includes Cannabis sativa flower, leaf, or stem extract.

Plant extracts may be obtained using maceration extraction, percolation, infusion, decoction, Soxhlet extraction, microwave-assisted extraction, sonication, ultrasound-assisted extraction, accelerated solvent extraction, supercritical fluid extraction, enzyme-assisted extraction, and solid phase microextraction, among other techniques.

### Formulations and Routes of Administration

In some embodiments, the composition (e.g., cosmetic composition) is formulated for topical administration to the skin of a human subject. In some embodiments, the composition (e.g., cosmetic composition) may be administered to any skin or exposed mucosal surface. Skin surfaces includes any part of the body, including but not limited to face, hands, legs, neck, abdominal area, eyes, nose, and chest. In some embodiments, the composition (e.g., cosmetic composition) can be in any form suitable for topical use such as, for example, an aerosol, dusting powder, jelly, patch, liquid, gel, lotion, paste, cream, foam, serum, ointment, or stick.

Creams are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Lotions are preparations to be applied to the skin surface without friction and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease with which a more fluid composition can cover large surfaces. It is generally desirable that the insoluble matter in a lotion be finely divided. Lotions will typically contain suspending agents to produce better dispersions as well as compounds useful for localizing and holding the active agent in contact with the skin.

Solutions are homogeneous mixtures prepared by dissolving one or more chemical substances (solutes) in a liquid such that the molecules of the dissolved substance are dispersed among those of the solvent. The solution may contain other acceptable chemicals to buffer, stabilize or preserve the solute. Common examples of solvents used in preparing solutions are ethanol, water, propylene glycol or any other acceptable vehicles.

Gels are semisolid, suspension-type systems. Single-phase gels contain organic macromolecules distributed substantially uniformly throughout the carrier liquid, which is typically aqueous, but also, preferably, contain an alcohol, and, optionally, an oil. In order to prepare a uniform gel, dispersing agents such as alcohol or glycerin can be added, or the gelling agent can be dispersed by trituration, mechanical mixing or stirring, or combinations thereof.

Ointments are semisolid preparations that are typically based on petrolatum or other petroleum derivatives. The specific ointment base to be used, as will be appreciated by those skilled in the art, is one that will provide for a number of desirable characteristics, such as emollience or the like. As with other carriers or vehicles, an ointment base may desirably be inert, stable, nonirritating, and nonsensitizing. As explained in Remington: The Science and Practice of Pharmacy, 19^{th} Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

In some embodiments, the composition (e.g., cosmetic composition) further includes an additive. Examples of additives include, but are not limited to, diluents, buffers, binders, surface-active agents, lubricants, humectants, pH adjusting agents, preservatives (including antioxidants), emulsifiers, occlusive agents, opacifiers, antioxidants, colorants, flavoring agents, gelling agents, thickening agents, stabilizers, and surfactants, among others.

### Methods of Using Compositions of the Disclosure to Improve Skin Quality and Outward Appearance

The compositions (e.g., cosmetic compositions) described herein may be used to improve the outward appearance of the skin of a human subject (e.g., a male or female human subject). Without being limited by mechanism, the outward appearance of the human subject may be improved, for example, by improving the subject's skin barrier functionality, thereby enhancing skin hydration and reducing transepidermal water loss. Transepidermal water loss in a subject (e.g., a male or female human subject) may be measured using a variety of methods known in the art, as described, for example, in Antonov et al., Curr. Probl. Dermatol. 49:61-70 (2016), the disclosure of which is incorporated herein by reference. In some embodiments, the composition (e.g., cosmetic composition) increases skin hydration, smooths skin, renews skin, brightens skin, and/or cleanses skin. In some embodiments, the disclosure provides methods for reducing redness in the skin of human subject.

In some embodiments, the compositions (e.g., cosmetic compositions) of the disclosure may be used to reduce redness in the skin of a subject (e.g., a male or female human subject). The redness may be caused, for example, by inflammation. Exemplary sources of inflammation include contact dermatitis, seborrheic dermatitis, nummular dermatitis, stasis dermatitis, atopic dermatitis, and dermatitis herpetiformis, among others. In some embodiments, the inflammation is due to exposure to ultraviolet (UV) radiation.

In some embodiments, the composition is administered to the subject one or more times (e.g., 2 times, 3 times, 4 times, 5 times, or more) daily. In some embodiments, the composition is administered to the subject once daily. In some embodiments, the composition is administered to the subject twice daily. In some embodiments, the composition is administered once in the morning and once in the evening. In some embodiments, the composition is administered to the subject three times daily. In some embodiments, the composition is administered to the subject twice in the morning and once at night. In some embodiments, the composition is administered to the subject for two or more continuous days. For example, the composition may be administered to the subject over the course of a period having a duration of from 2 continuous days to 30 continuous days, or more, such as a period having a duration of 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more. In some embodiments, the composition is administered to the subject for at least 7 continuous days (e.g., for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more). In some embodiments, the composition is administered to the subject for at least 14 continuous days (e.g., for 14 days, 15 days, 16 days, 17 days, 18 days, 19 days, 20 days, 21 days, 22 days, 23 days, 24 days, 25 days, 26 days, 27 days, 28 days, 29 days, 30 days, or more). In some embodiments, the composition is administered to the subject for at least 21 continuous days (e.g., for 21 days, 22 days, 23 days, 24 days, 25 days, 26 days, 27 days, 28 days, 29 days, 30 days, or more). In some embodiments, the composition is administered to the subject for at least 28 continuous days (e.g., for 21 days, 22 days, 23 days, 24 days, 25 days, 26 days, 27 days, 28 days, 29 days, 30 days, or more).

### Improving Skin Barrier Functionality

The disclosure provides methods for improving the skin barrier function by administering to a subject a composition (e.g., cosmetic composition) described herein. Human skin is composed of two compartments, namely a deep compartment, the dermis, and a superficial compartment, the epidermis. The epidermis is in contact with the external environment. Its role is to protect the body from dehydration and external aggressions, chemical, mechanical, or infectious. The compositions (e.g., cosmetic compositions) of the disclosure may be used to enhance the ability of the epidermis to act as an effective barrier to these external irritants.

The cells constituting the epidermis are delimited by an intercellular lipid structure. During differentiation, phospholipids—the role of which is to develop the fluid structure of the cell membranes of the living layers of the epidermis—are gradually replaced by a mixture composed mainly of fatty acids, cholesterol, and sphingolipids. These lipids are organized into specific lamellar structures whose integrity depends not only on the quality of the fractions present but also on their respective proportion. This lamellar structure of lipids is responsible for the appearance of suppleness of the skin. Among lipids, sphingolipids (ceramides) are essential for the maintenance of the multilamellar structure of intercorneocyte lipids, which are essential for water exchange and the barrier function of the epidermis. The stratum corneum provides the epidermal barrier to water loss from the skin. This barrier is analogous to a brick wall, with the corneocytes acting as the bricks and the lamellar lipids as the mortar. This barrier permits the retention of water within the corneocytes and as a result they swell up preventing the formation of cracks in the skin between them.

The intercorneocyte lipids undergo modifications. This maturation is necessary for the establishment of a good barrier function. The deglycosylation of lipid precursors like glucosylceramide into ceramide is modulated by the action of specific endogenous glycosidases (glucosidase). This deglycosylation is therefore an important step in the establishment of the barrier function of the skin.

The lipids of the skin, particularly of the epidermis, are influenced by genetic factors, aging, diets, environmental factors, aggressions and/or certain pathologies which can alter or even modify the composition of the lipids in the skin or reduce their quantity, leading to dry skin.

Improvement in the skin barrier function may thus reduce skin dryness, flakiness, dullness and/or redness that arises from transepidermal water loss. Consequently, improvements in skin barrier functionality and transepidermal water loss may result in an improved appearance of the skin.

### Reducing Redness Due to Skin Inflammation

In some embodiments, the compositions (e.g., cosmetic composition) of the disclosure may be used to reduces skin inflammation. Skin inflammation may be caused by dryness resulting from transepidermal water loss and reduced skin barrier functionality. Skin inflammation may, in turn, cause redness of the skin. In some embodiments, the skin inflammation is caused by dermatitis, psoriasis, rosacea, or exposure to UV radiation. In some embodiments, the dermatitis is contact dermatitis, seborrheic dermatitis, nummular dermatitis, statis dermatitis, atopic dermatitis, or dermatitis herpetiformis. In some embodiments, the skin inflammation may be chronic; for example, skin inflammation as a result of psoriasis may be chronic inflammation. In some embodiments, the skin inflammation may be acute; for example, skin inflammation as a result of exposure to UV radiation may be acute inflammation.

### Brightening the Skin

In some embodiments, the cosmetic compositions of the disclosure may be used to brighten the skin of a subject. In some embodiments, administering the cosmetic composition to the subject (e.g., male or female human subject) may result in visibly brightened skin, visibility more radiant skin, increased skin smoothness, increased evenness of skin tone, increased overall skin clarity, and/or diminished hyperpigmentation. In some embodiments, the skin may be dull due to an accumulation of dead skin cells on the surface of the skin. In some embodiments, the skin may be dull due to skin dryness that may arise from transepidermal water loss. In some embodiments, the skin dullness may be caused by dehydration. In some embodiments, the skin dullness may be caused by aging.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regards as their invention.

### Example 1. Evaluating the Ability of Cannabigerol to Improve Skin Barrier Functionality, Diminish Transepidermal Water Loss, and Reduce Inflammation-Induced Redness in the Skin of Human Subjects

### Summary of the Clinical Study

This example describes the results of a single center, single-blind clinical study in twenty-two healthy male and female human subjects that was conducted with the aim of investigating the skin protectant and anti-inflammatory properties of cannabigerol (CBG) on damaged skin. Particularly, the objective of the study was to evaluate the relative degree of improvement to skin barrier functionality and to determine the anti-inflammatory efficacy of three CBG-containing test articles, referred to herein as Test Article 1, Test Article 2, and Test Article 3, and a placebo article. The test articles and placebo article were evaluated against a test skin site following open application of an irritant. Assessment of transepidermal water loss (TEWL), grading of skin site, and evaluation of irritation were performed to determine the effects of the CBG-containing test articles on subjects' skin.

### Materials and Methods

### Study Design

Baseline skin barrier function was assessed, by way of a TEWL assay, on Day 0 in all subjects. Four volar forearm skin sites were observed in every subject in order to obtain the baseline skin barrier function score. After baseline evaluation, 0.75% w/v sodium lauryl sulfate (SLS) on a patch was applied to each of the four test sites for 24 hours to induce irritation. After 24 hours, on Day 1, the SLS patches were removed. Assessment of irritation and skin barrier function was then conducted to obtain a baseline measurement of these parameters.

Additionally, on Day 0, four test skin sites on each subject's back were exposed to ultraviolet radiation at four times the minimal erythemal dose (MED). The irradiated skin sites were evaluated for irritation and skin barrier function 24 hours after exposure to the UV irradiation.

After obtaining baseline measurements of skin barrier function and UV-induced irritation, Test Articles 1-3 and the Placebo Article were applied to the SLS- and UV-treated test skin sites on Day 1. The test and placebo articles were applied to the test sites for 10 minutes. At the conclusion of the 10-minute application period, the test skin sites were evaluated for TEWL and irritation. Test Articles 1-3 and the Placebo Article were then re-applied to the same sites a total of 9 applications throughout the duration of the 15-day study period. TEWL readings were taken on days 1, 4, 9, and 15. Clinical grading of the test sites was performed prior to application of the test articles on Day 0 (baseline), Day 1, Day 2, Day 3, Day 4, Day 7, Day 8, Day 9, Day 10, Day 11, and Day 14.

### Selection of Subjects

The subjects enrolled in this study were healthy males and females of age 18 years and older. Excluded from the trial were pregnant subjects and those undergoing lactation, women of child-bearing potential at risk of becoming pregnant, those having a current skin disease at the test site, those who exhibit heavy alcohol consumption in the opinion of the investigator, those having a fever in the 12 hours prior to the initial patch application, those having a significant medical history of hepatic, renal, cardiac, pulmonary, digestive, hematological, neurological, locomotor or psychiatric disease, those having a history of malignant disease, those having insulin-dependent or non-insulin-dependent diabetes, those taking a concurrent medication likely to affect the response to the test articles or confuse the results of the study, such as routine, high-dosage use of anti-inflammatory drugs (e.g., aspirin, ibuprofen, or corticosteroids), those having a known sensitivity to the treatment solutions or their constituents including patch materials, those exhibiting sensitization or questionable sensitization in a Repeat Insult Patch Test, and those that have used self-tanning lotion on the test area within one week prior to the start of the study.

Additionally, subjects were prepared to accept certain prohibitions and restrictions. Particularly, subjects agreed not to use aspirin or non-steroidal anti-inflammatory drugs for the duration of the trial. Subjects agreed not swim during the study or to deliberately expose the test sites to natural sunlight or to other sources of UV light during the study period. Subjects were not to receive an immunization during the study period, nor were subjects to use self-tanning lotion on the test area during the study. All subjects were informed of the nature, purpose, and known risks of the study both orally and in writing. Furthermore, all subjects were advised that they were free to withdraw from the study at any time without being obliged to give a reason. The study conformed to the requirements of the 1964 Declaration of Helsinki and its subsequent amendments (World Medical Association; 2013).

### Inflammation Induction Protocol

On Day 0, a 0.75% w/v SLS solution in water was applied to occlusive patches that consisted of a breathable tape with non-breathable adhesive and a central WEBRIL® pad portion of 2 cm by 2 cm on the volar forearm. The sites on the volar forearm were marked with skin markers. These SLS patches stayed in place for approximately 24 hours for the induction of an inflammatory skin response.

Additionally, on Day 0, each subject was treated with a series of five light exposures in order to determine the MED for unprotected skin. Each exposure time was 1.25 times greater than the previous one. This procedure continued until each subject produced a reaction equal to four times greater than their MED.

The MED study area was outlined on the lower back, between the waist and the scapula, and lateral to the midline. The test sites for UV-induced inflammation were outlined while the subjects sat upright in a backless chair. A template of the study area containing five subsites, each 3 cm×2.5 cm, was marked on the back. The template was located so as to ensure that no moles or skin lesions were present in any of these subsites. Only the subsites were exposed to UV radiation. A record of individual exposure times and subsites was kept. After UV exposure, any immediate skin responses were noted and subjects were instructed to keep the test area covered from sunlight or other sources of UV light for the next 24 hours before returning to the study center.

### Open Application Dosing Regime

On Day 1, subjects returned to the study center and had their SLS patches removed. TEWL assessments and clinical grading were conducted for each test site. Each subject had one control site, which was untreated with a test article or placebo article. The remaining sites on each subject were treated with an open application of the three test articles and the placebo article. The test articles and placebo articles were applied to each subject in an amount sufficient to physically cover each test site's surface area. The application areas were covered with a thin gauze pad, which was taped on all sides so that the test and placebo articles remained in place.

Approximately 10 minutes (±2 minutes) post-application, the gauze and test/placebo articles were removed, and each of the test sites was assessed for irritation and TEWL. Subsequently, the test and placebo articles were re-applied in the same manner and again covered with the gauze pad, with all sides taped to cover the applied areas. Subjects were instructed to keep the open application sites covered and the bandaging dry, and were given an appointment to return to the study center for the next study day.

On Days 4, 9, and 15, subjects returned to the testing facility approximately 24 hours after each application. The subjects had the article applications removed and the sites were gently wiped to remove any residual article. TEWL measurements were taken, and the test/placebo articles were then re-applied and covered as outlined above. Applications occurred on Days 1, 2, 3, 4, 7, 8, 9, 10, 11, and 14. The test/placebo articles were applied to the same sites throughout the duration of the study. Similarly, the control sites remained untreated throughout the study.

Clinical grading of the test sites was performed prior to the application of the test/placebo articles at baseline, post SLS removal, and on study Days 1, 2, 3, 4, 7, 8, 9, 10, 11, and 14 after article removal. On Day 15, subjects returned to the testing facility approximately 24 hours after the final article application, and the same measurements were conducted. After all assessments had been recorded, the study was considered complete.

### Assessments of Transepidermal Water Loss

TEWL measurements were performed using the TEWAMETER® TM300. The measurement of the water evaporation, and therefore TEWL, is based on the diffusion principle in an open chamber. The density gradient is measured indirectly by two pairs of sensors in the probe attachment, one for temperature and the other for relative humidity. This density gradient is then analyzed by a microprocessor in the instrument. A 15-minute warm-up period was allowed before using the TEWAMETER®. TEWL readings were taken at all test sites marked on the volar forearm on Day 0, before application of the SLS patches. TEWL readings were taken at the treated and untreated sites after removal of the SLS patches and 24 hours post-irradiation (MED) on Day 1, and again on all sites approximately 10 minutes (±2 minutes) after application of the test articles to three of the test sites and the placebo to one test site. TEWL readings were taken at each test site on Days 1, 4, and 9 after removal of the test articles and prior to re-application. Final TEWL readings were taken on Day 15 for all test sites.

### Skin Site Grading

Clinical grading of the test sites was performed prior to the application of SLS at on Day 0, after removal of the SLS patches on Day 1, 24 hours post-irradiation (MED) on Day 1, and 10 minutes (±2 minutes) after the first application of the articles on Day 1. Clinical grading also occurred prior to product application on Day 2, Day 3, Day 4, Day 7, Day 8, Day 9, Day 10, Day 11, and a final grading on Day 14. All test sites, for the duration of the study, were evaluated according to the scoring scale in Table 1. Illumination of the test sites was achieved using a 60-watt pearl bulb, approximately 30 cm from the site.

TABLE 1
Clinical Skin Grading Scale

| Score | Observations |
|---|---|
| 0.0 | No apparent cutaneous involvement |
| 0.5 | Faint, barely perceptible erythema or slight dryness (glazed appearance) |
| 1.0 | Faint but definite erythema, no eruptions or broken skin or No erythema but definite dryness; |
| | may have epidermal fissuring |
| 1.5 | Well-defined erythema or faint erythema with definite dryness, may have epidermal fissuring |
| 2.0 | Moderate erythema, may have a very few papules or deep fissures, moderate-to-severe |
| | erythema in the cracks |
| 2.5 | Moderate erythema with barely perceptible edema or severe erythema not involving a |
| | significant portion of the patch (halo effect around the edges), may have a few papules or |
| | moderate-to-severe erythema |
| 3.0 | Severe erythema (beet redness), may have generalized papules or moderate-to-severe |
| | erythema with slight edema (edges well defined by raising) |
| 3.5 | Moderate-to-severe erythema with moderate edema (confined to patch area) or moderate-to- |
| | severe erythema with isolated eschar formations or vesicles |
| 4.0 | Generalized vesicles or eschar formations or moderate-to-severe erythema and/or edema |
| | extending beyond the area of the patch |
| A | Tape reaction |
| E | Edema |
| P | Papules |
| S | Reaction spread |
| X | If a score greater than 1.5 is recorded, site is not re-patched and score recorded is residual |
| | score (not included in total scores) |
| — | — Subject did not attend (Scores for subjects not completing the study are not included |
| | in total scores) |

### Subject Demographics

The study enrolled 22 subjects of a mean age of 32.4 years (standard deviation: 9.9), a median age of 31.5 years. Subjects had an age range of from 18 to 51 years. Of the 22 subjects, 12 were female and 10 were male.

### Test Article and Placebo Compositions

The three test articles and the placebo article included the ingredients shown in Table 2.

TABLE 2
Article ingredients

| Article | Ingredients |
|---|---|
| Test Article 1 | Capric/Caprylic triglyceride and Cannabigerol |
| Test Article 2 | Squalane, Capric/Caprylic triglyceride, and Cannabigerol |
| Test Article 3 | Squalane, Capric/Caprylic Triglyceride, |
| | Cannabis Sativa Seed Oil, Cannabigerol, |
| | Cannabis Sativa Flower/Leaf/Stem Extract |
| | (Broad Spectrum), and Lavandula |
| | Angustifolia (lavender) Oil |
| Placebo Article | Capric/Caprylic triglyceride |

### Statistical Analysis Methods

To assess TEWL and visual irritancy, descriptive statistics (mean and standard deviation) were used. Changes from baseline measurements in TEWL and visual irritancy were assessed by way of paired T-Tests, mean percent improvements from baseline, and by calculating the percent of subjects improving. To evaluate the change from baseline between treatment analysis, analysis of covariance was used (ANOVA). All statistical tests of hypothesis employed a level of significance of 0.05 and no adjustments were made for the number of tests performed.

### Results

### Effects of Test Articles on Skin Barrier Function

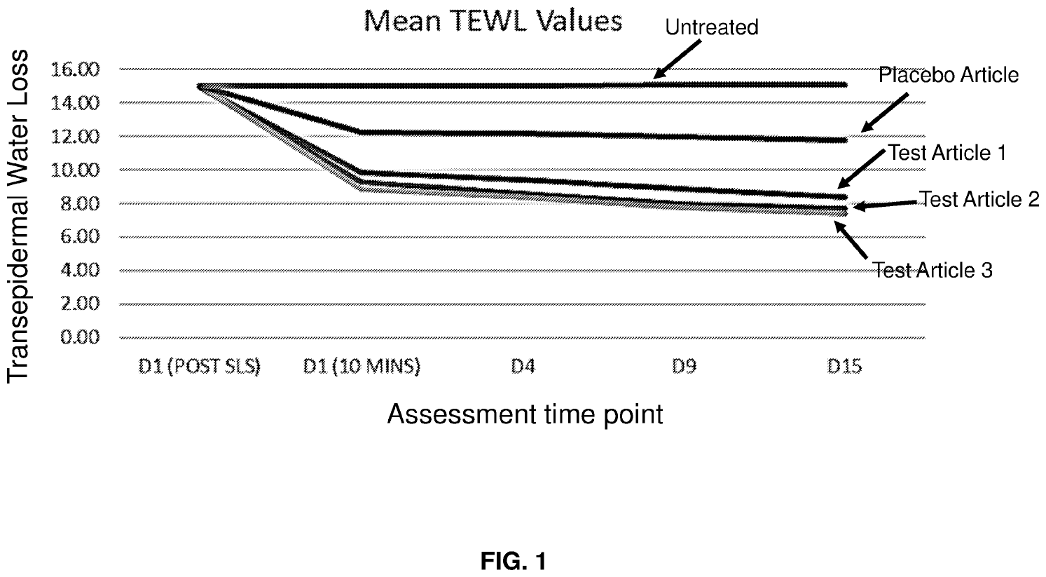

The skin barrier function was evaluated for each subject following administration of either Test Article 1, Test Article 2, Test Article 3, or Placebo Article, by assessing transepidermal water loss (in units of g/h/m²) at five separate time points (Baseline post SLS, 10 minutes after administration of article, Day 4, Day 9, and Day 15) (Table 3). Test Articles 1-3 all showed a reduction in transepidermal water loss of at least about 44% compared to the skin site at baseline post SLS prior to the first administration of the articles (Table 3 and FIG. 1).

TABLE 3
Mean TEWL values

| | | | | Mean | Within- | | | Between- |
|---|---|---|---|---|---|---|---|---|
| | | | | Difference | Treatment | Percent | Percent | Treatment |
| | | | Treatment | From | t-test | Change | Change | ANOVA |
| Treatment | Evaluation | N | Mean | Baseline | p-value | Mean | Subject | p-value |
| Test Article 1 | Baseline | 22 | 14.97 | | | | | |
| | Post SLS | | | | | | | |
| | 10 Minutes | 22 | 9.85 | −5.12 | <0.0001* | −34.2% | −34.1% | <0.0001** |
| | Day 4 | 22 | 9.39 | −5.58 | <0.0001* | −37.3% | −37.2% | <0.0001** |
| | Day 9 | 22 | 8.85 | −6.12 | <0.0001* | −40.9% | −40.8% | <0.0001** |
| | Day 15 | 22 | 8.41 | −6.56 | <0.0001* | −43.8% | −43.8% | <0.0001** |
| Test Article 2 | Baseline | 22 | 14.93 | | | | | |
| | Post SLS | | | | | | | |
| | 10 Minutes | 22 | 9.28 | −5.64 | <0.0001* | −37.8% | −37.8% | |
| | Day 4 | 22 | 8.58 | −6.35 | <0.0001* | −42.5% | −42.5% | |
| | Day 9 | 22 | 7.95 | −6.97 | <0.0001* | −46.7% | −46.7% | |
| | Day 15 | 22 | 7.69 | −7.23 | <0.0001* | −48.5% | −48.4% | |
| Test Article 3 | Baseline | 22 | 15.01 | | | | | |
| | Post SLS | | | | | | | |
| | 10 Minutes | 22 | 8.86 | −6.15 | <0.0001* | −41.0% | −40.9% | |
| | Day 4 | 22 | 8.40 | −6.61 | <0.0001* | −44.0% | −44.0% | |
| | Day 9 | 22 | 7.79 | −7.22 | <0.0001* | −48.1% | −48.1% | |
| | Day 15 | 22 | 7.43 | −7.58 | <0.0001* | −50.5% | −50.5% | |
| Placebo | Baseline | 22 | 14.99 | | | | | |
| Article | Post SLS | | | | | | | |
| | 10 Minutes | 22 | 12.28 | −2.71 | <0.0001* | −18.1% | −18.0% | |
| | Day 4 | 22 | 12.16 | −2.82 | <0.0001* | −18.8% | −18.7% | |
| | Day 9 | 22 | 11.99 | −3.00 | <0.0001* | −20.0% | −19.9% | |
| | Day 15 | 22 | 11.78 | −3.21 | <0.0001* | −21.4% | −21.3% | |
| Untreated | Baseline | 22 | 15.02 | | | | | |
| | Post SLS | | | | | | | |
| | 10 Minutes | 22 | 15.01 | −0.00 | >0.5000 | −0.0% | −0.0% | |
| | Day 4 | 22 | 15.03 | 0.01 | 0.0261* | 0.1% | 0.1% | |
| | Day 9 | 22 | 15.06 | 0.05 | <0.0001* | 0.3% | 0.3% | |
| | Day 15 | 22 | 15.08 | 0.07 | <0.0001* | 0.4% | 0.4% | |

In Table 3, a single asterisk denotes a significant change from baseline. A double asterisk denotes a significant difference between treatments.

Furthermore, when the TEWL values for skin sites treated with one of the 4 articles (Test Article 1, Test Article 2, Test Article 3, or Placebo Article) were compared to the untreated skin sites for 10 minutes post first treatment, on Day 4, on Day 9, and on Day 15 using Tukey-Kramer Multiple Comparison statistical testing, greater differences were observed between Test Article-treated sites and untreated control sites than with Placebo Article (Table 4). Furthermore, using Dunnett's Multiple Comparison statistical analysis, a significant difference was found between treatment 1 (Test Articles or Placebo) and treatment 2 (Untreated) (Table 4).

TABLE 4
Tukey-Kramer Multiple Comparison for Test Article, Placebo, and Untreated Sites

| | | | Estimate Difference | Between-Treatment |
|---|---|---|---|---|
| TEWL Assessment | | | Between Treatments | Dunnett's Test |
| Time Point | Treatment 1 | Treatment 2 | From ANOVA | p-value |
| 10 Minutes | Test Article 1 | Untreated | −5.160 | <0.0001** |
| Day 4 | Test Article 1 | Untreated | −5.645 | <0.0001** |
| Day 9 | Test Article 1 | Untreated | −6.212 | <0.0001** |
| Day 15 | Test Article 1 | Untreated | −6.671 | <0.0001** |
| 10 Minutes | Test Article 2 | Untreated | −5.729 | <0.0001** |
| Day 4 | Test Article 2 | Untreated | −6.457 | <0.0001** |
| Day 9 | Test Article 2 | Untreated | −7.115 | <0.0001** |
| Day 15 | Test Article 2 | Untreated | −7.391 | <0.0001** |
| 10 Minutes | Test Article 3 | Untreated | −6.154 | <0.0001** |
| Day 4 | Test Article 3 | Untreated | −6.628 | <0.0001** |
| Day 9 | Test Article 3 | Untreated | −7.273 | <0.0001** |
| Day 15 | Test Article 3 | Untreated | −7.652 | <0.0001** |
| 10 Minutes | Placebo Article | Untreated | −2.736 | <0.0001** |
| Day 4 | Placebo Article | Untreated | −2.869 | <0.0001** |
| Day 9 | Placebo Article | Untreated | −3.074 | <0.0001** |
| Day 15 | Placebo Article | Untreated | −3.300 | <0.0001** |

In Table 4, a double asterisk denotes a significant difference between Treatment 1 and Treatment

### Effects of Test Articles on Visual Irritancy

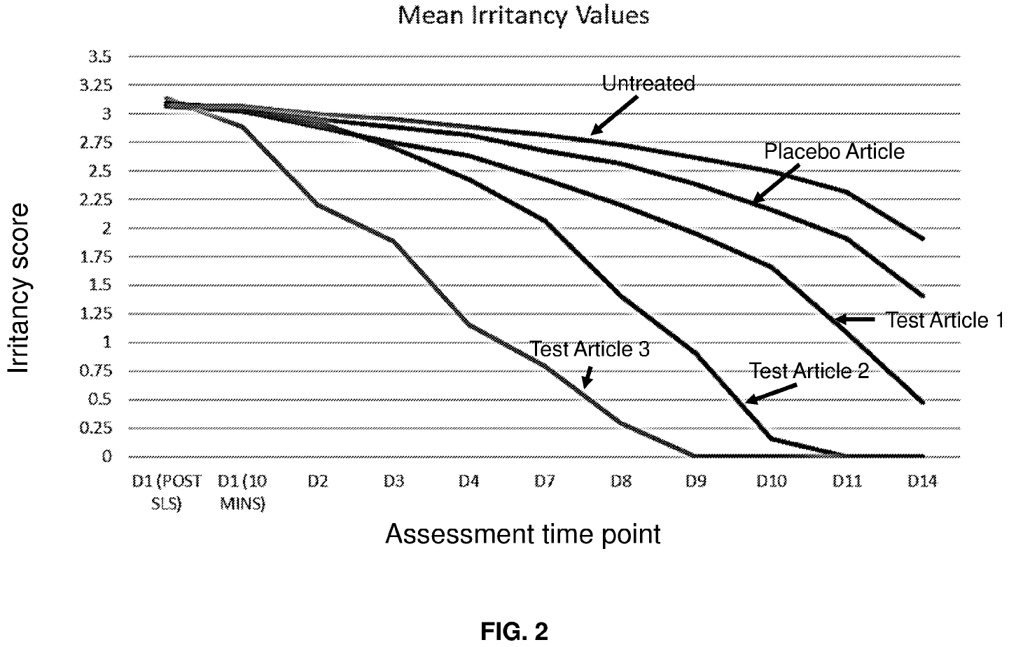

The irritation scores of the test skin sites after exposure to SLS and after administration of Test Article 1, Test Article 2, Test Article 3, or Placebo Article are reported in Table 5 and FIG. 2. In Table 5, a double asterisk denotes a statistically significant difference.

TABLE 5
Visual irritancy scores

| | | | | Mean | Within- | | | | | Between- |
|---|---|---|---|---|---|---|---|---|---|---|
| | | | | Difference | Treatment | Mean | Percent | | | Treatment |
| | | | Treatment | From | t-test | Percent | Change | Number | Percent | ANOVA |
| Treatment | Evaluation | N | Mean | Baseline | p-value | Change | Subject | Improved | Improved | p-value |
| Test | Baseline | 22 | 3.07 | | | | | | | |
| Article 1 | Post SLS | | | | | | | | | |
| | 10 Minutes | 22 | 3.02 | −0.05 | 0.1621 | −1.5% | −1.3% | 2 | 9.1% | <0.0001** |
| | Day 2 | 22 | 2.89 | −0.18 | 0.0079* | −5.9% | −5.4% | 7 | 31.8% | <0.0001** |
| | Day 3 | 22 | 2.75 | −0.32 | 0.0002* | −10.4% | −9.8% | 12 | 54.5% | <0.0001** |
| | Day 4 | 22 | 2.64 | −0.43 | <0.0001* | −14.1% | −13.6% | 17 | 77.3% | <0.0001** |
| | Day 7 | 22 | 2.43 | −0.64 | <0.0001* | −20.7% | −20.2% | 19 | 86.4% | <0.0001** |
| | Day 8 | 22 | 2.20 | −0.86 | <0.0001* | −28.1% | −27.4% | 20 | 90.9% | <0.0001* |
| | Day 9 | 22 | 1.95 | −1.11 | <0.0001* | −36.3% | −35.6% | 22 | 100.0% | <0.0001** |
| | Day 10 | 22 | 1.66 | 1.41 | <0.0001* | −45.9% | −45.2% | 22 | 100.0% | <0.0001** |
| | Day 11 | 22 | 1.09 | −1.98 | <0.0001* | −64.4% | −63.9% | 22 | 100.0% | <0.0001** |
| | Day 14 | 22 | 0.48 | −2.59 | <0.0001* | −84.4% | −83.7% | 22 | 100.0% | <0.0001** |
| Test | Baseline | 22 | 3.09 | | | | | | | |
| Article 2 | Post SLS | | | | | | | | | |
| | 10 Minutes | 22 | 3.02 | −0.07 | 0.0829 | −2.2% | −1.9% | 3 | 13.6% | |
| | Day 2 | 22 | 2.93 | −0.16 | 0.0051* | −5.1% | −4.7% | 7 | 31.8% | |
| | Day 3 | 22 | 2.70 | 0.39 | <0.0001* | −12.5% | −11.7% | 13 | 59.1% | |
| | Day 4 | 22 | 2.43 | −0.66 | <0.0001* | −21.3% | −20.7% | 20 | 90.9% | |
| | Day 7 | 22 | 2.07 | −1.02 | <0.0001* | −33.1% | −32.5% | 21 | 95.5% | |
| | Day 8 | 22 | 1.41 | −1.68 | <0.0001* | −54.4% | −53.9% | 22 | 100.0% | |
| | Day 9 | 22 | 0.91 | −2.18 | <0.0001* | −70.6% | −70.2% | 22 | 100.0% | |
| | Day 10 | 22 | 0.16 | −2.93 | <0.0001* | −94.9% | −94.5% | 22 | 100.0% | |
| | Day 11 | 22 | 0.00 | −3.09 | <0.0001* | 100.0% | −100.0% | 22 | 100.0% | |
| | Day 14 | 22 | 0.00 | −3.09 | <0.0001* | 100.0% | 100.0% | 22 | 100.0% | |
| Test | Baseline | 22 | 3.14 | | | | | | | |
| Article 3 | Post SLS | | | | | | | | | |
| | 10 Minutes | 22 | 2.89 | −0.25 | 0.0002* | −8.0% | −7.6% | 11 | 50.0% | |
| | Day 2 | 22 | 2.20 | −0.93 | <0.0001* | −29.7% | −29.3% | 22 | 100.0% | |
| | Day 3 | 22 | 1.89 | 1.25 | <0.0001* | −39.9% | −39.3% | 22 | 100.0% | |
| | Day 4 | 22 | 1.16 | −1.98 | <0.0001* | −63.0% | −62.4% | 22 | 100.0% | |
| | Day 7 | 22 | 0.80 | 2.34 | <0.0001* | −74.6% | −74.3% | 22 | 100.0% | |
| | Day 8 | 22 | 0.30 | −2.84 | <0.0001* | −90.6% | −90.4% | 22 | 100.0% | |
| | Day 9 | 22 | 0.00 | −3.14 | <0.0001* | 100.0% | −100.0% | 22 | 100.0% | |
| | Day 10 | 22 | 0.00 | −3.14 | <0.0001* | 100.0% | −100.0% | 22 | 100.0% | |
| | Day 11 | 22 | 0.00 | −3.14 | <0.0001* | 100.0% | 100.0% | 22 | 100.0% | |
| | Day 14 | 22 | 0.00 | −3.14 | <0.0001* | 100.0% | −100.0% | 22 | 100.0% | |
| Placebo | Baseline | 22 | 3.09 | | | | | | | |
| Article | Post SLS | | | | | | | | | |
| | 10 Minutes | 22 | 3.05 | −0.05 | 0.1621 | −1.5% | −1.3% | 2 | 9.1% | |
| | Day 2 | 22 | 2.95 | −0.14 | 0.0106* | −4.4% | 3.9% | 6 | 27.3% | |
| | Day 3 | 22 | 2.89 | −0.20 | 0.0038* | −6.6% | −6.0% | 8 | 36.4% | |
| | Day 4 | 22 | 2.82 | −0.27 | 0.0003* | −8.8% | −8.2% | 11 | 50.0% | |
| | Day 7 | 22 | 2.68 | 0.41 | <0.0001* | −13.2% | −12.6% | 15 | 68.2% | |
| | Day 8 | 22 | 2.57 | −0.52 | <0.0001* | −16.9% | −16.2% | 16 | 72.7% | |
| | Day 9 | 22 | 2.39 | 0.70 | <0.0001* | −22.8% | −21.9% | 20 | 90.9% | |
| | Day 10 | 22 | 2.16 | −0.93 | <0.0001* | −30.1% | −29.5% | 22 | 100.0% | |
| | Day 11 | 22 | 1.91 | −1.18 | <0.0001* | −38.2% | −37.3% | 22 | 100.0% | |
| | Day 14 | 22 | 1.41 | −1.68 | <0.0001* | −54.4% | −53.9% | 22 | 100.0% | |
| Untreated | Baseline | 22 | 3.07 | | | | | | | |
| | Post SLS | | | | | | | | | |
| | 10 Minutes | 22 | 3.07 | 0.00 | | 0.0% | 0.0% | 0 | 0.0% | |
| | Day 2 | 22 | 3.00 | −0.07 | 0.0829 | −2.2% | −1.9% | 3 | 13.6% | |
| | Day 3 | 22 | 2.95 | −0.11 | 0.0215* | −3.7% | −3.4% | 5 | 22.7% | |
| | Day 4 | 22 | 2.89 | −0.18 | 0.0023* | −5.9% | −5.4% | 8 | 36.4% | |
| | Day 7 | 22 | 2.82 | −0.25 | 0.0022* | −8.1% | −7.5% | 9 | 40.9% | |
| | Day 8 | 22 | 2.73 | −0.34 | <0.0001* | −11.1% | −10.8% | 13 | 59.1% | |
| | Day 9 | 22 | 2.61 | −0.45 | <0.0001* | −14.8% | −14.4% | 15 | 68.2% | |
| | Day 10 | 22 | 2.50 | −0.57 | <0.0001* | −18.5% | −18.1% | 18 | 81.8% | |
| | Day 11 | 22 | 2.32 | −0.75 | <0.0001* | −24.4% | −23.9% | 21 | 95.5% | |
| | Day 14 | 22 | 1.91 | −1.16 | <0.0001* | −37.8% | −37.2% | 22 | 100.0% | |

Additionally, multiple comparison analyses for skin irritation were performed to assess skin sites treated with one of the 4 articles (Test Article 1, Test Article 2, Test Article 3, or Placebo Article). The comparisons were made using Tukey-Kramer Multiple Comparison statistical testing (Table 6). Greater differences between Test Article-treated sites and untreated control sites were observed relative to the differences observed between Placebo Article-treated sites and untreated control sites.

TABLE 6
Tukey-Kramer Multiple Comparison for Test
Article, Placebo, and Untreated Sites

| | | | Estimate Difference | Between-Treatment |
|---|---|---|---|---|
| | | | Between Treatments | Dunnett's Test |
| Evaluation | Treatment 1 | Treatment 2 | From ANOVA | p-value |
| 10 Minutes | Test Article 1 | Untreated | −0.045 | >0.5000 |
| Day 2 | Test Article 1 | Untreated | −0.114 | 0.2129 |
| Day 3 | Test Article 1 | Untreated | −0.205 | 0.0173** |
| Day 4 | Test Article 1 | Untreated | −0.250 | 0.0050** |
| Day 7 | Test Article 1 | Untreated | −0.386 | <0.0001** |
| Day 8 | Test Article 1 | Untreated | −0.523 | <0.0001** |
| Day 9 | Test Article 1 | Untreated | −0.659 | <0.0001** |
| Day 10 | Test Article 1 | Untreated | −0.841 | <0.0001** |
| Day 11 | Test Article 1 | Untreated | −1.227 | <0.0001** |
| Day 14 | Test Article 1 | Untreated | −1.432 | <0.0001** |
| 10 Minutes | Test Article 2 | Untreated | −0.064 | 0.4605 |
| Day 2 | Test Article 2 | Untreated | −0.080 | >0.5000 |
| Day 3 | Test Article 2 | Untreated | −0.257 | 0.0018** |
| Day 4 | Test Article 2 | Untreated | −0.464 | <0.0001** |
| Day 7 | Test Article 2 | Untreated | −0.753 | <0.0001** |
| Day 8 | Test Article 2 | Untreated | −1.316 | <0.0001** |
| Day 9 | Test Article 2 | Untreated | −1.701 | <0.0001** |
| Day 10 | Test Article 2 | Untreated | −2.339 | <0.0001** |
| Day 11 | Test Article 2 | Untreated | −2.316 | <0.0001** |
| Day 14 | Test Article 2 | Untreated | −1.907 | <0.0001** |
| 10 Minutes | Test Article 3 | Untreated | −0.238 | <0.0001** |
| Day 2 | Test Article 3 | Untreated | −0.832 | <0.0001** |
| Day 3 | Test Article 3 | Untreated | −1.090 | <0.0001** |
| Day 4 | Test Article 3 | Untreated | −1.755 | <0.0001** |
| Day 7 | Test Article 3 | Untreated | −2.031 | <0.0001** |
| Day 8 | Test Article 3 | Untreated | −2.426 | <0.0001** |
| Day 9 | Test Article 3 | Untreated | −2.603 | <0.0001** |
| Day 10 | Test Article 3 | Untreated | −2.493 | <0.0001** |
| Day 11 | Test Article 3 | Untreated | −2.311 | <0.0001** |
| Day 14 | Test Article 3 | Untreated | −1.903 | <0.0001** |
| 10 Minutes | Placebo Article | Untreated | −0.041 | >0.5000 |
| Day 2 | Placebo Article | Untreated | −0.058 | >0.5000 |
| Day 3 | Placebo Article | Untreated | −0.076 | >0.5000 |
| Day 4 | Placebo Article | Untreated | −0.077 | >0.5000 |
| Day 7 | Placebo Article | Untreated | −0.139 | 0.2305 |
| Day 8 | Placebo Article | Untreated | −0.157 | 0.2931 |
| Day 9 | Placebo Article | Untreated | −0.224 | 0.0118** |
| Day 10 | Placebo Article | Untreated | −0.339 | 0.0001** |
| Day 11 | Placebo Article | Untreated | −0.407 | <0.0001** |
| Day 14 | Placebo Article | Untreated | −0.498 | <0.0001** |

### Adverse Events

No adverse events or reactions were reported at any point during the study. All subjects completed the study.

### Conclusion

Taken together, the data collected from this study demonstrate that Test Articles 1-3 are effective at reducing epidermal irritation and restoring skin barrier function.

### Example 2. Use of CBG for Reducing Skin Redness Due to Inflammation

Using the compositions and methods of the disclosure, a cosmetic composition containing CBG may be administered to the skin of a human subject (e.g., a male or female human subject) so as to reduce redness in the subject's skin. The redness may be caused, for example, by inflammation, such as contact dermatitis, seborrheic dermatitis, nummular dermatitis, stasis dermatitis, atopic dermatitis, and dermatitis herpetiformis, among others. Additionally or alternatively, the inflammation may be due to exposure to UV radiation.

To reduce redness in the subject's skin, the cosmetic composition may be applied to the affected area of the skin topically. The composition may be formulated, for instance, as a patch, aerosol, dusting powder, jelly, patch, liquid, gel, lotion, paste, cream, foam, serum, ointment, or stick, among other forms that are suitable for application directly to a skin site. The composition may be applied in a volume sufficient to physically cover the entirety of the affected area.

In some instances, it may be desirable to re-apply the composition to the affected skin site one or more times (e.g., 2 times, 3 times, 4 times, 5 times, or more) daily until the desired level of improvement in skin redness is achieved. For example, the composition may be administered to the subject once daily for two or more continuous days. In some instances, the composition may be administered to the subject over the course of a period having a duration of from 2 continuous days to 30 continuous days, or more, such as a period having a duration of 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more.

For example, in some instances, the composition is administered to the subject for at least 7 continuous days (e.g., for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more). In some instances, the composition is administered to the subject for at least 14 continuous days (e.g., for 14 days, 15 days, 16 days, 17 days, 18 days, 19 days, 20 days, 21 days, 22 days, 23 days, 24 days, 25 days, 26 days, 27 days, 28 days, 29 days, 30 days, or more). In some instances, the composition is administered to the subject for at least 21 continuous days (e.g., for 21 days, 22 days, 23 days, 24 days, 25 days, 26 days, 27 days, 28 days, 29 days, 30 days, or more).

At the conclusion of the administration period, the subject may assess skin redness by visual inspection so as to determine whether a desirable degree of improvement has been achieved. If further reduction of redness is desired, the subject may continue to re-apply the composition to the affected skin site for a second or subsequent administration period.

### Example 3. Use of CBG for Improving Skin Barrier Function

Using the compositions and methods of the disclosure, a cosmetic composition containing CBG may be administered to the skin of a human subject (e.g., a male or female human subject) so as to improve the barrier functionality of a subject's skin. This may be performed, for example, with the aim of reducing transepidermal water loss so as to augment the skin's hydration. In this way, the composition may be administered to the subject's skin so as to improve its outward appearance by reducing flakiness, fine lines, and other blemishes associated with epidermal dehydration.

To improve the skin barrier functionality of a subject, the cosmetic composition may be applied to the affected area of the subject's skin topically. The composition may be formulated, for instance, as a patch, aerosol, dusting powder, jelly, patch, liquid, gel, lotion, paste, cream, foam, serum, ointment, or stick, among other forms that are suitable for application directly to a skin site. The composition may be applied in a volume sufficient to physically cover the entirety of the affected area.

In some instances, it may be desirable to re-apply the composition to the affected skin site one or more times (e.g., 2 times, 3 times, 4 times, 5 times, or more) daily until the desired level of improvement in skin barrier functionality is achieved. For example, the composition may be administered to the subject once daily for two or more continuous days. In some instances, the composition may be administered to the subject over the course of a period having a duration of from 2 continuous days to 30 continuous days, or more, such as a period having a duration of 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more.

For example, in some instances, the composition is administered to the subject for at least 7 continuous days (e.g., for 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more). In some instances, the composition is administered to the subject for at least 14 continuous days (e.g., for 14 days, 15 days, 16 days, 17 days, 18 days, 19 days, 20 days, 21 days, 22 days, 23 days, 24 days, 25 days, 26 days, 27 days, 28 days, 29 days, 30 days, or more). In some instances, the composition is administered to the subject for at least 21 continuous days (e.g., for 21 days, 22 days, 23 days, 24 days, 25 days, 26 days, 27 days, 28 days, 29 days, 30 days, or more).

At the conclusion of the administration period, the subject may assess skin barrier functionality by way of a TEWL assay (e.g., a TEWL assay described in Example 1, above) and/or by way of visual inspection so as to determine whether a desirable degree of improvement has been achieved. If further improvement of skin barrier functionality is desired, the subject may continue to re-apply the composition to the affected skin site for a second or subsequent administration period.

### Example 4. Evaluating the Ability of Cannabigerol to Improve Erythema, Lesion Thickness, Flaking and Scaling Across all Psoriatic Lesions in the Skin of Human Subjects

### Summary of the Clinical Study

This example describes the results of a home-use, single-blind, multi-site clinical study in thirty-five male and female human subjects with mild-to-moderate psoriasis that was conducted with the aim of investigating the effects of CBG on skin damaged by psoriatic lesions. Particularly, the objective of the study was to evaluate the efficacy and safety of three CBG-containing test articles, referred to herein as Test Article 1, Test Article 2, and Test Article 3, and a placebo article. The test articles and placebo article were evaluated against a psoriatic lesion site. Assessment of lesion sites included evaluation of the following variables: erythema, induration (lesion thickness), flaking, and scaling.

### Materials and Methods

### Study Design and Procedure

Potential subjects visited the testing facility on day 0 of the study, and a trained evaluator assessed the psoriasis lesions of each subject. Informed consent and a pre-treatment questionnaire were obtained if the subject was accepted into the study. A clinically trained evaluator graded the identified test site which was followed at subsequent visits. Sites were graded against a 6-point scale, evaluating the following variables: erythema, induration (lesion thickness), flaking, and scaling across all psoriatic lesions (all psoriatic lesions were identified and documented in the study files). An average erythema, induration, flaking, and scaling score was obtained over the whole body according to a 6-point scale 0 (Clear)-5 (Severe). The total score was calculated as an average of the two severity scores and rounded to the nearest whole number score to determine the sPGA score. In this way, severity was categorized as: 0=Clear; 1=Minimal; 2=Mild; 3=Moderate; 4=Marked; or 5=Severe. Assessments were taken at baseline, Day 7, Day 14, and Day 28. Subjects were asked to rate the severity of their psoriasis at baseline, Day 7, Day 14, and Day 28. Clinical Photography images were taken of the identified test sites at Baseline, Day 7, Day 14, and Day 28. Self-perception questionnaires were completed at Day 7, Day 14, and Day 28. Subjects were issued their corresponding test article or placebo article to use daily, once in the evening, during the 4-week test period. Study assessments were conducted according to Table 7, below. Thirty-five subjects were studied split into four groups. The study duration was four weeks. After cleansing of the psoriatic lesion site, subjects applied 3-5 drops of Test Article 1, Test Article 2, Test Article 3, or Placebo Article over the affected area. Subjects were instructed to avoid eye area and to use the test article in the morning and in the evening.

TABLE 7
Timeline of study of CBG on psoriatic lesions

| | Study Time Point | | | |
|---|---|---|---|---|
| | Baseline | Day | Day | Day |
| | Day 0 | 7 | 14 | 28 |
| | Visit | | | |
| | 1 | 2 | 3 | 4 |
| Informed Consent | ✓ | | | |
| Inclusion/Exclusion Criteria | ✓ | | | |
| Psoriatic Lesions Count | ✓ | ✓ | ✓ | ✓ |
| Erythema, Induration (Lesion thickness), Scaling and Flaking | ✓ | ✓ | ✓ | ✓ |
| Visual Grades | | | | |
| Clinical Photography | ✓ | ✓ | ✓ | ✓ |
| Subject Self-Assessment | ✓ | ✓ | ✓ | ✓ |
| Test Article Distribution/Collection | ✓ | | | ✓ |
| Supervised Application | ✓ | | | |
| Self-Perception Questionnaire | | ✓ | ✓ | ✓ |
| Test Article Compliance Check | | ✓ | ✓ | ✓ |
| Adverse Event Review | ✓ | ✓ | ✓ | ✓ |

### Selection of Subjects

The subjects enrolled in this study satisfied the following inclusion criteria. First, all subjects were healthy males and females of age 18 years and older with mild to moderate psoriasis either through previous medical diagnosis or through self-reporting. Second, subjects had skin types within Fitzpatrick skin types I through V. Third, all subjects were willing to follow the study instructions and were available to attend the study visits. Lastly, all subjects were willing to provide written informed consent and to sign a photography release. Excluded from the trial were: pregnant subjects and those undergoing lactation, women of child-bearing potential at risk of becoming pregnant, those having known allergy or hypersensitivity to acne treatment products containing sulfur, those having any conditions on the test site that would interfere with evaluations (i.e. tattoos, scars, open cuts, sunburn, piercings, excessive hair, etc.), those having used depigmenting medications, such as hydroquinone, during the 14 days prior to the start of the study, those with insulin-dependent diabetes, those taking medication that might affect the response to the test articles including routine use of anti-inflammatory medications, anti-histamines, and steroids, those with a history of Crohn's disease, or clinically significant enteritis (regional enteritis, ulcerative colitis, pseudomembranous colitis, antibiotic-associated colitis), those with microdermabrasion or laser treatment in the test area within six months of the study, those with a medical condition which, in the opinion of the Investigator, would compromise the safety of the subject or confound study results, and those having participated in an investigational drug study within 4 months of the baseline visit.

Additionally, subjects were prepared to accept certain prohibitions and restrictions. Particularly, subjects agreed, during the 4-week treatment period, to continue to use their usual skin cleansing products. Subjects also agreed to attend all visits with a clean test area (no make-up or skincare products such as moisturizer, sunscreen, etc.). Subjects agreed to not consume any hot or caffeinated foods or beverages or smoke within one hour of study visits. Lastly, subjects agreed to protect the test area from excessive sun exposure, tanning, burning during the 4-week study duration.

All subjects were informed of the nature, purpose, and known risks of the study both orally and in writing. Furthermore, all subjects were advised that they were free to withdraw from the study at any time without being obliged to give a reason. The study conformed to the requirements of the 1964 Declaration of Helsinki and its subsequent amendments (World Medical Association; 2013).

### Assessments

A trained evaluator monitored the following attributes (erythema, induration (lesion thickness), flaking, and scaling) to assess the severity of the subject's psoriasis (Table 8).

TABLE 8
Assessment scale of psoriatic lesions
Global Assessment Scale

| | |
|---|---|
| 0 | Clear |
| 1 | Minimal |
| 2 | Mild |
| 3 | Moderate |
| 4 | Marked |
| 5 | Severe |

Subjects were also shown the above scale (Table 8) and asked to rate the severity of psoriasis at baseline, day 7, day 14 and day 27.

### Clinical Photography

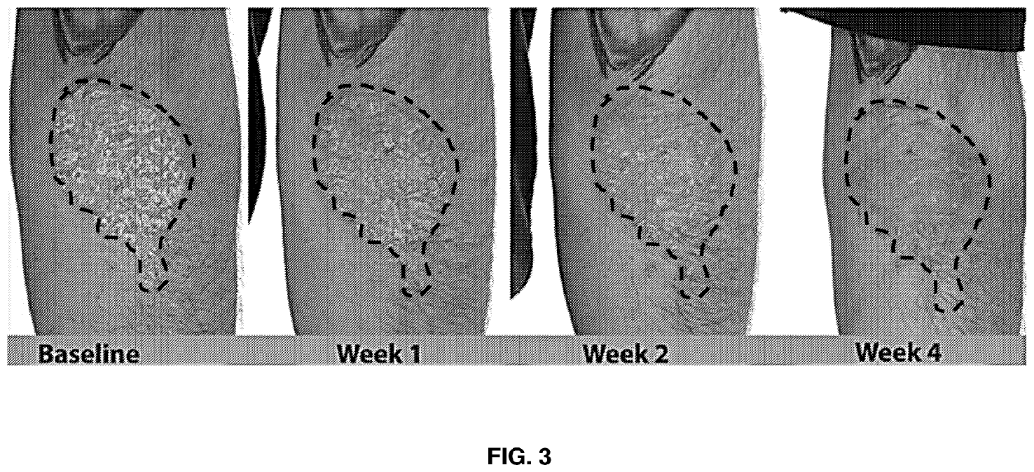

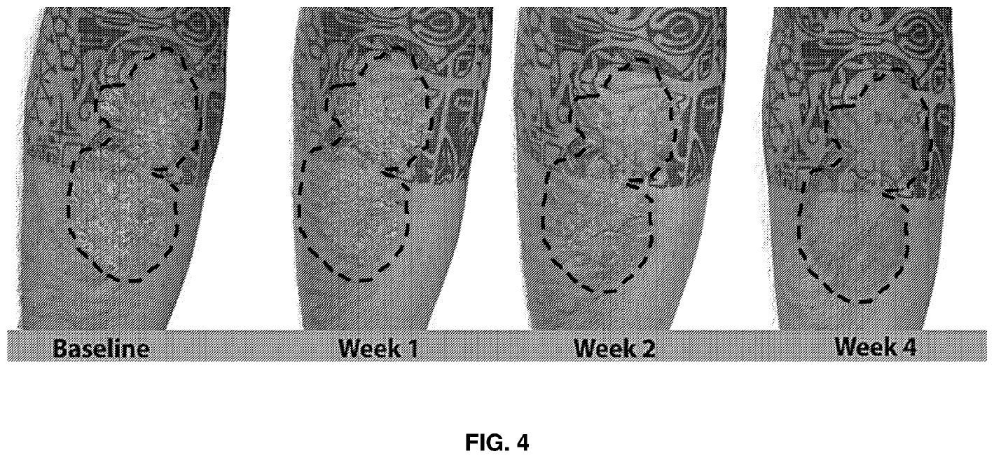

High resolution digital images were taken of the identified test site at baseline, day 7, day 14 and day 28 for each subject (FIGS. 3-4). The following equipment and settings were used to capture the images: Nikon D5500 camera, Nikon 18-140 mm lens, all images captured with camera raw at ISO 200 1/125^{th} @ F20, 4X Soft box @135 W main light, camera mounted fill light speed lite, remote background light speed lite.

### Test Article and Placebo Compositions

The three test articles and the placebo article included the ingredients shown in Table 9.

TABLE 9
Article ingredients

| Article | Ingredients |
|---|---|
| Test Article 1 | Capric/Caprylic triglyceride and Cannabigerol |
| Test Article 2 | Squalane, Capric/Caprylic triglyceride, and Cannabigerol |
| Test Article 3 | Squalane, Capric/Caprylic Triglyceride, |
| | Cannabis Sativa Seed Oil, Cannabigerol, |
| | Cannabis Sativa Flower/Leaf/Stem Extract |
| | (Broad Spectrum), and Lavandula |
| | Angustifolia (lavender) Oil |
| Placebo Article | Capric/Caprylic triglyceride |

### Statistical Analysis Methods

The source data including visual and clinical assessments were analyzed using both a between-treatment analysis (One-way ANOVA) to measure the differences between the three treatment products and the placebo. Within-treatment analysis were conducted to analyze on changes from baseline utilizing Wilcoxon's signed rank test. Percent changes from baseline data were also provided. Self-Perception Questionnaire responses were summarized specifying percent agreement for each SPQ statement (with Strongly Agree and Agree as the top box scores). All statistical tests of hypothesis employed a level of significance of 0.05 and no adjustments were made for the number of tests performed. A formal sample size calculation was not required for this study. Enrolling an adequate number of subjects to complete with about 35 subjects was considered sufficient to be able to make certain claims from the data collected.

### Results

### Effects of Test Articles on Psoriatic Lesions

As to the safety of Test Articles 1-3 and the Placebo Article, no adverse reactions were reported in all 35 subjects that completed the study. The efficacy of Test Articles 1-3 and Placebo Article was evaluated for each subject following administration of either Test Article 1, Test Article 2, Test Article 3, or Placebo Article by visual inspection at four separate time points (Baseline Day 0, Day 7, Day 14, and Day 28) (Tables 10-17).

Upon visual inspection, subjects treated with each test article showed improvements in erythema using the 6-point severity score (Tables 10 and 11) following 28 consecutive days of use (p<0.001).

TABLE 10
Erythema severity scores at multiple time points following visual inspection
Within-Treatment

| | | | | | Within-Treatment | Percent |
|---|---|---|---|---|---|---|
| | | | Treatment | Mean Difference | Signed Rank | Change |
| Attribute | Evaluation | N | Mean | From Baseline | p-value | Mean |
| Placebo | Baseline | 23 | 2.5870 | | | |
| | Day 7 | 23 | 2.6087 | 0.22 | 1.00 | 0.84% |
| | Day 14 | 23 | 2.6522 | 0.65 | 1.00 | 2.52% |
| | Day 28 | 23 | 2.6739 | 0.87 | 1.00 | 3.36% |
| Test Article 1 | Baseline | 25 | 2.1200 | | | |
| | Day 7 | 24 | 1.5417 | 0.58 | 0.10 | −27.28% |
| | Day 14 | 24 | 1.1667 | 0.95 | <0.001* | −44.97% |
| | Day 28 | 24 | 0.6458 | 1.47 | <0.001* | −69.54% |
| Test Article 2 | Baseline | 21 | 2.2143 | | | |
| | Day 7 | 21 | 1.5238 | 0.69 | 0.113 | −31.18% |
| | Day 14 | 21 | 0.8571 | 1.36 | <0.001* | −61.29% |
| | Day 28 | 21 | 0.4286 | 1.79 | <0.001* | −80.65% |
| Test Article 3 | Baseline | 21 | 2.5714 | | | |
| | Day 7 | 21 | 1.6429 | 0.93 | 0.016* | −36.11% |
| | Day 14 | 21 | 0.7857 | 1.79 | <0.001* | −69.44% |
| | Day 28 | 21 | 0.2619 | 2.31 | <0.001* | −89.81% |

* Significant change from baseline

TABLE 11
Erythema severity difference between test articles
and placebo article at multiple time points
Test Article 1, Test Article 2, Test Article 3 vs
Placebo Article (Between treatment analysis)

| Test article | Timepoint | p-value |
|---|---|---|
| Test Article 1 | Baseline | 0.771 |
| | Day 7 | <0.001* |
| | Day 14 | <0.001* |
| | Day 28 | <0.001* |
| Test Article 2 | Baseline | 1.000 |
| | Day 7 | 0.001* |
| | Day 14 | <0.001* |
| | Day 28 | <0.001* |
| Test Article 2 | Baseline | 1.000 |
| | Day 7 | 0.005* |
| | Day 14 | <0.001* |
| | Day 28 | <0.001* |

*Significant change compared to the placebo article at the given time point.

Upon visual inspection, subjects treated with each test article showed improvements in induration using the 6-point severity score (Tables 12 and 13) following 28 consecutive days of use (p<0.001).

TABLE 12
Induration severity scores at multiple time points following visual inspection

| | | | | | Within-Treatment | Percent |
|---|---|---|---|---|---|---|
| | | | Treatment | Mean Difference | Signed Rank | Change |
| Attribute | Evaluation | N | Mean | From Baseline | p-value | Mean |
| Placebo Article | Baseline | 23 | 2.0870 | | | |
| | Day 7 | 23 | 2.2174 | 0.13 | 1.00 | 6.25% |
| | Day 14 | 23 | 2.3261 | 0.24 | 1.00 | 11.46% |
| | Day 28 | 23 | 2.2174 | 0.13 | 1.00 | 6.25% |
| Test Article 1 | Baseline | 25 | 2.1000 | | | |
| | Day 7 | 24 | 1.1458 | 0.95 | <0.001* | −45.44% |
| | Day 14 | | 240.5208 | 1.58 | <0.001* | −75.20% |
| | Day 28 | 24 | 0.2708 | 1.83 | <0.001* | −87.10% |
| Test Article 2 | Baseline | 21 | 2.3095 | | | |
| | Day 7 | 21 | 1.4048 | 0.90 | 0.008 | −39.18% |
| | Day 14 | 21 | 10.7381 | 1.57 | <0.001* | −68.04% |
| | Day 28 | 21 | 10.3095 | 2.00 | <0.001* | −86.60% |
| Test Article 2 | Baseline | 21 | 13.0714 | | | |
| | Day 7 | 21 | 1.9286 | 1.14 | <0.001* | −37.21% |
| | Day 14 | 21 | 0.8333 | 2.24 | <0.001* | −72.87% |
| | Day 28 | 21 | 0.2381 | 2.83 | <0.001* | −92.25% |

* Significant change from baseline

TABLE 13
Induration severity difference between test articles
and placebo article at multiple time points
Test Article 1, Test Article 2, Test Article 3 vs
Placebo Article (Between treatment analysis)

| Test article | Timepoint | p-value |
|---|---|---|
| Test Article 1 | Baseline | 1.000 |
| | Day 7 | 0.001* |
| | Day 14 | <0.001* |
| | Day 28 | <0.001* |
| Test Article 2 | Baseline | 1.000 |
| | Day 7 | 0.030 |
| | Day 14 | <0.001* |
| | Day 28 | <0.001* |
| Test Article 2 | Baseline | 0.004 |
| | Day 7 | 1.000 |
| | Day 14 | <0.001* |
| | Day 28 | <0.001* |

*Significant change compared to the placebo article at the given time point.

Upon visual inspection, subjects treated with each test article showed improvements in flaking using the 6-point severity score (Tables 14 and 15) following 28 consecutive days of use (p<0.001).

TABLE 14
Flaking severity scores at multiple time points following visual inspection

| | | | | | Within-Treatment | Percent |
|---|---|---|---|---|---|---|
| | | | Treatment | Mean Difference | Signed Rank | Change |
| Attribute | Evaluation | N | Mean | From Baseline | p-value | Mean |
| Placebo Article | Baseline | 23 | 1.8043 | | | |
| | Day 7 | 23 | 1.7826 | 0.02 | 1.00 | −1.20% |
| | Day 14 | 23 | 1.7174 | 0.07 | 1.00 | −4.82% |
| | Day 28 | 23 | 1.7609 | 0.43 | 1.00 | −2.41% |
| Test Article 1 | Baseline | 25 | 1.4000 | | | |
| | Day 7 | 24 | 0.6667 | 0.73 | 0.009 | −52.38% |
| | Day 14 | 24 | 0.3333 | 1.07 | <0.001* | −76.19% |
| | Day 28 | 24 | 0.1458 | 1.25 | <0.001* | −89.58% |
| Test Article 2 | Baseline | 21 | 1.5476 | | | |
| | Day 7 | 21 | 0.6429 | 0.90 | <0.001* | −58.46% |
| | Day 14 | 21 | 0.2619 | 1.29 | <0.001* | −83.08% |
| | Day 28 | 21 | 0.1190 | 1.43 | <0.001* | −92.31% |
| Test Article 2 | Baseline | 21 | 1.6429 | | | |
| | Day 7 | 21 | 0.7619 | 0.88 | 0.006* | −53.62% |
| | Day 14 | 21 | 0.2143 | 1.43 | <0.001* | −86.96% |
| | Day 28 | 21 | 0.0476 | 1.60 | <0.001* | −97.10% |

* Significant change from baseline.

TABLE 15
Flaking severity difference between test articles
and placebo article at multiple time points
Test Article 1, Test Article 2, Test Article 3 vs
Placebo Article (Between treatment analysis)

| Test article | Timepoint | p-value |
|---|---|---|
| Test Article 1 | Baseline | 1.000 |
| | Day 7 | <0.001* |
| | Day 14 | <0.001* |
| | Day 28 | <0.001* |
| Test Article 2 | Baseline | 1.000 |
| | Day 7 | <0.001* |
| | Day 14 | <0.001* |
| | Day 28 | <0.001* |
| Test Article 2 | Baseline | 1.000 |
| | Day 7 | 0.001 |
| | Day 14 | <0.001* |
| | Day 28 | <0.001* |

*Significant change compared to the placebo article at the given time point.

Upon visual inspection, subjects treated with each test article showed improvements in scaling using the 6-point severity score (Tables 16 and 17) following 28 consecutive days of use (p<0.001).

TABLE 16
Scaling severity scores at multiple time points following visual inspection
Within-Treatment Percent

| | | | | | Within-Treatment | Percent |
|---|---|---|---|---|---|---|
| | | | Treatment | From Baseline | Signed Rank | Change |
| Attribute | Evaluation | N | Mean | Mean Difference | p-value | Mean |
| Placebo Article | Baseline | 23 | 1.8261 | | | |
| | Day 7 | 23 | 1.7391 | 0.09 | 1.00 | −4.76% |
| | Day 14 | 23 | 1.6739 | 0.15 | 1.00 | −8.33% |
| | Day 28 | 23 | 1.6739 | 0.15 | 1.00 | −8.33% |
| Test Article 1 | Baseline | 25 | 1.6600 | | | |
| | Day 7 | | 240.8750 | 0.79 | 0.002 | −47.29% |
| | Day 14 | 24 | 0.5208 | 1.14 | <0.001* | −68.62% |
| | Day 28 | 24 | 0.2708 | 1.39 | <0.001* | −83.68% |
| Test Article 2 | Baseline | 21 | 1.7619 | | | |
| | Day 7 | 21 | 0.8810 | 0.88 | 0.017 | −50.00% |
| | Day 14 | 21 | 0.5714 | 1.19 | <0.001* | −67.57% |
| | Day 28 | 21 | 0.3810 | 1.38 | <0.001* | −78.38% |
| Test Article 2 | Baseline | 21 | 2.1190 | | | |
| | Day 7 | 21 | 1.2619 | 0.86 | 0.010 | −40.45% |
| | Day 14 | 21 | 0.7619 | 1.36 | <0.001* | −64.04% |
| | Day 28 | 21 | 0.2619 | 1.86 | <0.001* | −87.64% |

* Significant change from baseline

TABLE 17
Scaling severity difference between test articles
and placebo article at multiple time points
Test Article 1, Test Article 2, Test Article 3 vs
Placebo Article (Between treatment analysis)

| Test article | Timepoint | p-value |
|---|---|---|
| Test Article 1 | Baseline | 1.000 |
| | Day 7 | 0.012 |
| | Day 14 | <0.001* |
| | Day 28 | <0.001* |
| Test Article 2 | Baseline | 1.000 |
| | Day 7 | 0.018 |
| | Day 14 | <0.001* |
| | Day 28 | <0.001* |
| Test Article 2 | Baseline | 1.000 |
| | Day 7 | 0.561 |
| | Day 14 | 0.002* |
| | Day 28 | <0.001* |

*Significant change compared to the placebo article at the given time point.

### Self-Perception of Psoriatic Lesions

A self-perception questionnaire (SPQ), shown in Table 18, was also provided to each group of subjects (those treated with Test Article 1, Test Article 2, Test Article 3, and Placebo Article) to assess the severity of the psoriatic lesions at Day 7, Day 14, and Day 28.

TABLE 18
Self-perception questionnaire

| | | | | | |
|---|---|---|---|---|---|
| | Strongly | | Neither agree | | Strongly |
| Questions | Agree | Agree | nor disagree | Disagree | Disagree |

I noticed less red patches on my
skin within “X” week using this
product.
My skin was less irritated within
“X” week using this product.
My skin felt less itchy within “X”
week using this product.
The psoriasis on my skin was
reduced within “X” week using this
product.
My skin felt less dry within “X”
week using this product.
The amount of skin flaking and
scaling has reduced from using
this product.
This product reduces the
occurrence of red patches on my
skin.
The number of lesions has
reduced since using the product.
The inflammation on my psoriasis
has reduced since using this
product.
This product is calming to my
skin.
This product improves the texture
of my skin.
This product makes my skin
feeling more hydrated and
smooth.
This product is soothing to my
skin.
This product helps control the
symptoms of psoriasis.
This product softens dry, rough
and patchy skin.
This product makes my skin feel
healthier.
I feel more confident and happier
with the use of this product.
I am satisfied with the
performance of this product.
I would purchase this product.
I would recommend this product
to a close friend or family
member.

Responses to the SPQ are summarized in Tables 19-30. Responses of “Strongly Agree” and “Agree” were counted as “top box” scores.

TABLE 19
SPQ Responses on Day 7 after daily use of Placebo Article

| Placebo Article | | Top Box | | | | |
|---|---|---|---|---|---|---|
| | Score for | Not Top Box | | Top Box | | |
| Question | Top Box | N | Percent | N | Percent | p-value |
| 1. | 4.5 | 20 | 87.0 | 3 | 13.0 | <0.001* |
| 2. | 4.5 | 13 | 56.5 | 10 | 43.5 | 0.532 |
| 3. | 4.5 | 12 | 52.2 | 11 | 47.8 | 0.835 |
| 4. | 4.5 | 20 | 87.0 | 3 | 13.0 | <0.001* |
| 5. | 4.5 | 7 | 30.4 | 16 | 69.6 | 0.061 |
| 6. | 4.5 | 13 | 56.5 | 10 | 43.5 | 0.532 |
| 7. | 4.5 | 20 | 87.0 | 3 | 13.0 | <0.001* |
| 8. | 4.5 | 19 | 82.6 | 4 | 17.4 | 0.002* |
| 9. | 4.5 | 20 | 87.0 | 3 | 13.0 | <0.001* |
| 10. | 4.5 | 12 | 52.2 | 11 | 47.8 | 0.835 |
| 11. | 4.5 | 10 | 43.5 | 13 | 56.5 | 0.532 |
| 12. | 4.5 | 5 | 21.7 | 18 | 78.3 | 0.007* |
| 13. | 4.5 | 16 | 69.6 | 7 | 30.4 | 0.061 |
| 14. | 4.5 | 19 | 82.6 | 4 | 17.4 | 0.002* |
| 15. | 4.5 | 10 | 43.5 | 13 | 56.5 | 0.532 |
| 16. | 4.5 | 16 | 69.6 | 7 | 30.4 | 0.061 |
| 17. | 4.5 | 20 | 87.0 | 3 | 13.0 | <0.001* |
| 18. | 4.5 | 16 | 69.6 | 7 | 30.4 | 0.061 |
| 19. | 4.5 | 17 | 73.9 | 6 | 26.1 | 0.022* |
| 20. | 4.5 | 14 | 60.9 | 9 | 39.1 | 0.297 |

TABLE 20
SPQ Responses on Day 7 after daily use of Test Article 1

| | | Top Box | | | | |
|---|---|---|---|---|---|---|
| Test Article 1 | | Not Top Box | | Top Box | | |
| Question | Score for Top Box | N | Percent | N | Percent | p-value |
| 1. | 4.5 | 11 | 44.0 | 14 | 56.0 | 0.549 |
| 2. | 4.5 | 6 | 24.0 | 19 | 76.0 | 0.009* |
| 3. | 4.5 | 5 | 20.0 | 20 | 80.0 | 0.003* |
| 4. | 4.5 | 11 | 44.0 | 14 | 56.0 | 0.549 |
| 5. | 4.5 | 6 | 24.0 | 19 | 76.0 | 0.009* |
| 6. | 4.5 | 8 | 32.0 | 17 | 68.0 | 0.072 |
| 7. | 4.5 | 12 | 48.0 | 13 | 52.0 | 0.841 |
| 8. | 4.5 | 11 | 44.0 | 14 | 56.0 | 0.549 |
| 9. | 4.5 | 11 | 44.0 | 14 | 56.0 | 0.549 |
| 10. | 4.5 | 7 | 28.0 | 18 | 72.0 | 0.028* |
| 11. | 4.5 | 5 | 20.0 | 20 | 80.0 | 0.003* |
| 12. | 4.5 | 3 | 12.0 | 22 | 88.0 | <0.001* |
| 13. | 4.5 | 9 | 36.0 | 16 | 64.0 | 0.162 |
| 14. | 4.5 | 10 | 40.0 | 15 | 60.0 | 0.317 |
| 15. | 4.5 | 9 | 36.0 | 16 | 64.0 | 0.162 |
| 16. | 4.5 | 7 | 28.0 | 18 | 72.0 | 0.028* |
| 17. | 4.5 | 12 | 48.0 | 13 | 52.0 | 0.841 |
| 18. | 4.5 | 9 | 36.0 | 16 | 64.0 | 0.162 |
| 19. | 4.5 | 9 | 36.0 | 16 | 64.0 | 0.162 |
| 20. | 4.5 | 8 | 32.0 | 17 | 68.0 | 0.072 |

*Significant

TABLE 21
SPQ Responses on Day 7 after daily use of Test Article 2

| | | Top Box | | | | |
|---|---|---|---|---|---|---|
| Test Article 2 | | Not Top Box | | Top Box | | |
| Question | Score for Top Box | N | Percent | N | Percent | p-value |
| 1. | 4.5 | 6 | 28.6 | 15 | 71.4 | 0.050* |
| 2. | 4.5 | 2 | 9.5 | 19 | 90.5 | <0.001* |
| 3. | 4.5 | 1 | 4.8 | 20 | 95.2 | <0.001* |
| 4. | 4.5 | 2 | 9.5 | 19 | 90.5 | <0.001* |
| 5. | 4.5 | 0 | 0.0 | 21 | 100.0 | <0.001* |
| 6. | 4.5 | 2 | 9.5 | 19 | 90.5 | <0.001* |
| 7. | 4.5 | 5 | 23.8 | 16 | 76.2 | 0.016* |
| 8. | 4.5 | 9 | 42.9 | 12 | 57.1 | 0.513 |
| 9. | 4.5 | 2 | 9.5 | 19 | 90.5 | <0.001* |
| 10. | 4.5 | 0 | 0.0 | 21 | 100.0 | <0.001* |
| 11. | 4.5 | 0 | 0.0 | 21 | 100.0 | <0.001* |
| 12. | 4.5 | 0 | 0.0 | 21 | 100.0 | <0.001* |
| 13. | 4.5 | 0 | 0.0 | 21 | 100.0 | <0.001* |
| 14. | 4.5 | 3 | 14.3 | 18 | 85.7 | 0.001* |
| 15. | 4.5 | 0 | 0.0 | 21 | 100.0 | <0.001* |
| 16. | 4.5 | 0 | 0.0 | 21 | 100.0 | <0.001* |
| 17. | 4.5 | 3 | 14.3 | 18 | 85.7 | 0.001* |
| 18. | 4.5 | 1 | 4.8 | 20 | 95.2 | <0.001* |
| 19. | 4.5 | 0 | 0.0 | 21 | 100.0 | <0.001* |
| 20. | 4.5 | 0 | 0.0 | 21 | 100.0 | <0.001* |

*Significant

TABLE 22
SPQ Responses on Day 7 after daily use of Test Article 3

| | | Top Box | | | | |
|---|---|---|---|---|---|---|
| Test Article 3 | | Not Top Box | | Top Box | | |
| Question | Score for Top Box | N | Percent | N | Percent | p-value |
| 1. | 4.5 | 2 | 9.5 | 19 | 90.5 | <0.001* |
| 2. | 4.5 | 2 | 9.5 | 19 | 90.5 | <0.001* |
| 3. | 4.5 | 2 | 9.5 | 19 | 90.5 | <0.001* |
| 4. | 4.5 | 1 | 4.8 | 20 | 95.2 | <0.001* |
| 5. | 4.5 | 1 | 4.8 | 20 | 95.2 | <0.001* |
| 6. | 4.5 | 4 | 19.0 | 17 | 81.0 | 0.005* |
| 7. | 4.5 | 5 | 23.8 | 16 | 76.2 | 0.016 |
| 8. | 4.5 | 7 | 33.3 | 14 | 66.7 | 0.127 |
| 9. | 4.5 | 3 | 14.3 | 18 | 85.7 | 0.001* |
| 10. | 4.5 | 1 | 4.8 | 20 | 95.2 | <0.001 |
| 11. | 4.5 | 0 | 0.0 | 21 | 100.0 | <0.001* |
| 12. | 4.5 | 1 | 4.8 | 20 | 95.2 | <0.001* |
| 13. | 4.5 | 0 | 0.0 | 21 | 100.0 | <0.001* |
| 14. | 4.5 | 2 | 9.5 | 19 | 90.5 | <0.001* |
| 15. | 4.5 | 1 | 4.8 | 20 | 95.2 | <0.001* |
| 16. | 4.5 | 1 | 4.8 | 20 | 95.2 | <0.001* |
| 17. | 4.5 | 3 | 14.3 | 18 | 85.7 | 0.001* |
| 18. | 4.5 | 2 | 9.5 | 19 | 90.5 | <0.001* |
| 19. | 4.5 | 2 | 9.5 | 19 | 90.5 | <0.001* |
| 20. | 4.5 | 1 | 4.8 | 20 | 95.2 | <0.001* |

*Significant

TABLE 23
SPQ Responses on Day 14 after daily use of Placebo Article

| | | Top Box | | | | |
|---|---|---|---|---|---|---|
| Placebo Article | | Not Top Box | | Top Box | | |
| Question | Score for Top Box | N | Percent | N | Percent | p-value |
| 1. | 4.5 | 9 | 39.1 | 14 | 60.9 | 0.297 |
| 2. | 4.5 | 3 | 13.0 | 20 | 87.0 | <0.001 |
| 3. | 4.5 | 4 | 17.4 | 19 | 82.6 | 0.002* |
| 4. | 4.5 | 9 | 39.1 | 14 | 60.9 | 0.297 |
| 5. | 4.5 | 1 | 4.3 | 22 | 95.7 | <0.001* |
| 6. | 4.5 | 5 | 21.7 | 18 | 78.3 | 0.007* |
| 7. | 4.5 | 9 | 39.1 | 14 | 60.9 | 0.297 |
| 8. | 4.5 | 11 | 47.8 | 12 | 52.2 | 0.835 |
| 9. | 4.5 | 9 | 39.1 | 14 | 60.9 | 0.297 |
| 10. | 4.5 | 4 | 17.4 | 19 | 82.6 | 0.002* |
| 11. | 4.5 | 4 | 17.4 | 19 | 82.6 | 0.002* |
| 12. | 4.5 | 2 | 8.7 | 21 | 91.3 | <0.001* |
| 13. | 4.5 | 4 | 17.4 | 19 | 82.6 | 0.002* |
| 14. | 4.5 | 7 | 30.4 | 16 | 69.6 | 0.061 |
| 15. | 4.5 | 5 | 21.7 | 18 | 78.3 | 0.007* |
| 16. | 4.5 | 7 | 30.4 | 16 | 69.6 | 0.061 |
| 17. | 4.5 | 8 | 34.8 | 15 | 65.2 | 0.144 |
| 18. | 4.5 | 7 | 30.4 | 16 | 69.6 | 0.061 |
| 19. | 4.5 | 7 | 30.4 | 16 | 69.6 | 0.061 |
| 20. | 4.5 | 7 | 30.4 | 16 | 69.6 | 0.061 |

*Significant

TABLE 24
SPQ Responses on Day 14 after daily use of Test Article 1

| | | Top Box | | | | |
|---|---|---|---|---|---|---|
| Test Article 1 | | Not Top Box | | Top Box | | |
| Question | Score for Top Box | N | Percent | N | Percent | p-value |
| 1. | 4.5 | 10 | 40.0 | 15 | 60.0 | 0.317 |
| 2. | 4.5 | 7 | 28.0 | 18 | 72.0 | 0.028* |
| 3. | 4.5 | 3 | 12.0 | 22 | 88.0 | <0.001* |
| 4. | 4.5 | 7 | 28.0 | 18 | 72.0 | 0.028* |
| 5. | 4.5 | 4 | 16.0 | 21 | 84.0 | <0.001* |
| 6. | 4.5 | 3 | 12.0 | 22 | 88.0 | <0.001* |
| 7. | 4.5 | 10 | 40.0 | 15 | 60.0 | 0.317 |
| 8. | 4.5 | 10 | 40.0 | 15 | 60.0 | 0.317 |
| 9. | 4.5 | 8 | 32.0 | 17 | 68.0 | 0.072 |
| 10. | 4.5 | 4 | 16.0 | 21 | 84.0 | <0.001* |
| 11. | 4.5 | 3 | 12.0 | 22 | 88.0 | <0.001* |
| 12. | 4.5 | 1 | 4.0 | 24 | 96.0 | <0.001* |
| 13. | 4.5 | 5 | 20.0 | 20 | 80.0 | 0.003* |
| 14. | 4.5 | 7 | 28.0 | 18 | 72.0 | 0.028* |
| 15. | 4.5 | 4 | 16.0 | 21 | 84.0 | <0.001* |
| 16. | 4.5 | 6 | 24.0 | 19 | 76.0 | 0.009* |
| 17. | 4.5 | 9 | 36.0 | 16 | 64.0 | 0.162 |
| 18. | 4.5 | 5 | 20.0 | 20 | 80.0 | 0.003* |
| 19. | 4.5 | 6 | 24.0 | 19 | 76.0 | 0.009* |
| 20. | 4.5 | 5 | 20.0 | 20 | 80.0 | 0.003* |

*Significant

TABLE 25
SPQ Responses on Day 14 after daily use of Test Article 2

| | | Top Box | | | | |
|---|---|---|---|---|---|---|
| Test Article 2 | | Not Top Box | | Top Box | | |
| Question | Score for Top Box | N | Percent | N | Percent | p-value |
| 1. | 4.5 | 8 | 38.1 | 13 | 61.9 | 0.275 |
| 2. | 4.5 | 2 | 9.5 | 19 | 90.5 | <0.001* |
| 3. | 4.5 | 2 | 9.5 | 19 | 90.5 | <0.001* |
| 4. | 4.5 | 5 | 23.8 | 16 | 76.2 | 0.016* |
| 5. | 4.5 | 0 | 0.0 | 21 | 100.0 | <0.001* |
| 6. | 4.5 | 2 | 9.5 | 19 | 90.5 | <0.001* |
| 7. | 4.5 | 8 | 38.1 | 13 | 61.9 | 0.275 |
| 8. | 4.5 | 10 | 47.6 | 11 | 52.4 | 0.827 |
| 9. | 4.5 | 8 | 38.1 | 13 | 61.9 | 0.275 |
| 10. | 4.5 | 2 | 9.5 | 19 | 90.5 | <0.001* |
| 11. | 4.5 | 1 | 4.8 | 20 | 95.2 | <0.001* |
| 12. | 4.5 | 0 | 0.0 | 21 | 100.0 | <0.001* |
| 13. | 4.5 | 4 | 19.0 | 17 | 81.0 | 0.005* |
| 14. | 4.5 | 5 | 23.8 | 16 | 76.2 | 0.016* |
| 15. | 4.5 | 3 | 14.3 | 18 | 85.7 | 0.001* |
| 16. | 4.5 | 2 | 9.5 | 19 | 90.5 | <0.001* |
| 17. | 4.5 | 8 | 38.1 | 13 | 61.9 | 0.275 |
| 18. | 4.5 | 5 | 23.8 | 16 | 76.2 | 0.016* |
| 19. | 4.5 | 4 | 19.0 | 17 | 81.0 | 0.005* |
| 20. | 4.5 | 3 | 14.3 | 18 | 85.7 | 0.001* |

*Significant

TABLE 26
SPQ Responses on Day 14 after daily use of Test Article 3

| | | Top Box | | | | |
|---|---|---|---|---|---|---|
| Test Article 3 | | Not Top Box | | Top Box | | |
| Question | Score for Top Box | N | Percent | N | Percent | p-value |
| 1. | 4.5 | 6 | 28.6 | 15 | 71.4 | 0.050* |
| 2. | 4.5 | 1 | 4.8 | 20 | 95.2 | <0.001* |
| 3. | 4.5 | 1 | 4.8 | 20 | 95.2 | <0.001* |
| 4. | 4.5 | 6 | 28.6 | 15 | 71.4 | 0.050* |
| 5. | 4.5 | 2 | 9.5 | 19 | 90.5 | <0.001* |
| 6. | 4.5 | 5 | 23.8 | 16 | 76.2 | 0.016* |
| 7. | 4.5 | 7 | 33.3 | 14 | 66.7 | 0.127 |
| 8. | 4.5 | 7 | 33.3 | 14 | 66.7 | 0.127 |
| 9. | 4.5 | 6 | 28.6 | 15 | 71.4 | 0.050* |
| 10. | 4.5 | 2 | 9.5 | 19 | 90.5 | <0.001* |
| 11. | 4.5 | 2 | 98.5 | 19 | 90.5 | <0.001* |
| 12. | 4.5 | 1 | 4.8 | 20 | 95.2 | <0.001* |
| 13. | 4.5 | 1 | 4.8 | 20 | 95.2 | <0.001* |
| 14. | 4.5 | 5 | 23.8 | 16 | 76.2 | 0.016* |
| 15. | 4.5 | 1 | 4.8 | 20 | 95.2 | <0.001* |
| 16. | 4.5 | 3 | 14.3 | 18 | 85.7 | 0.001* |
| 17. | 4.5 | 4 | 19.0 | 17 | 81.0 | 0.005* |
| 18. | 4.5 | 4 | 19.0 | 17 | 81.0 | 0.005* |
| 19. | 4.5 | 4 | 19.0 | 17 | 81.0 | 0.005* |
| 20. | 4.5 | 3 | 14.3 | 18 | 85.7 | 0.001* |

*Significant

TABLE 27
SPQ Responses on Day 28 after daily use of Placebo Article

| | | Top Box | | | | |
|---|---|---|---|---|---|---|
| Placebo Article | | Not Top Box | | Top Box | | |
| Question | Score for Top Box | N | Percent | N | Percent | p-value |
| 1. | 4.5 | 8 | 34.8 | 15 | 65.2 | 0.144 |
| 2. | 4.5 | 3 | 13.0 | 20 | 87.0 | <0.001 |
| 3. | 4.5 | 3 | 13.0 | 20 | 87.0 | <0.001 |
| 4. | 4.5 | 8 | 34.8 | 15 | 65.2 | 0.144 |
| 5. | 4.5 | 2 | 8.7 | 21 | 91.3 | <0.001 |
| 6. | 4.5 | 5 | 21.7 | 18 | 78.3 | 0.007 |
| 7. | 4.5 | 8 | 34.8 | 15 | 65.2 | 0.144 |
| 8. | 4.5 | 10 | 43.5 | 13 | 56.5 | 0.532 |
| 9. | 4.5 | 9 | 39.1 | 14 | 60.9 | 0.297 |
| 10. | 4.5 | 4 | 17.4 | 19 | 82.6 | 0.002 |
| 11. | 4.5 | 4 | 17.4 | 19 | 82.6 | 0.002 |
| 12. | 4.5 | 2 | 8.7 | 21 | 91.3 | <0.001 |
| 13. | 4.5 | 4 | 17.4 | 19 | 82.6 | 0.002 |
| 14. | 4.5 | 7 | 30.4 | 16 | 69.6 | 0.061 |
| 15. | 4.5 | 5 | 21.7 | 18 | 78.3 | 0.007 |
| 16. | 4.5 | 7 | 30.4 | 16 | 69.6 | 0.061 |
| 17. | 4.5 | 7 | 30.4 | 16 | 69.6 | 0.061 |
| 18. | 4.5 | 7 | 30.4 | 16 | 69.6 | 0.061 |
| 19. | 4.5 | 7 | 30.4 | 16 | 69.6 | 0.061 |
| 20. | 4.5 | 7 | 30.4 | 16 | 69.6 | 0.061 |

*Significant

TABLE 28
SPQ Responses on Day 28 after daily use of Test Article 1

| | | Top Box | | | | |
|---|---|---|---|---|---|---|
| Test Article 1 | | Not Top Box | | Top Box | | |
| Question | Score for Top Box | N | Percent | N | Percent | p-value |
| 1. | 4.5 | 9 | 36.0 | 16 | 64.0 | 0.162 |
| 2. | 4.5 | 6 | 24.0 | 19 | 76.0 | 0.009* |
| 3. | 4.5 | 2 | 8.0 | 23 | 92.0 | <0.001* |
| 4. | 4.5 | 7 | 28.0 | 18 | 72.0 | 0.028* |
| 5. | 4.5 | 3 | 12.0 | 22 | 88.0 | <0.001* |
| 6. | 4.5 | 3 | 12.0 | 22 | 88.0 | <0.001* |
| 7. | 4.5 | 9 | 36.0 | 16 | 64.0 | 0.162 |
| 8. | 4.5 | 9 | 36.0 | 16 | 64.0 | 0.162 |
| 9. | 4.5 | 8 | 32.0 | 17 | 68.0 | 0.072 |
| 10. | 4.5 | 4 | 16.0 | 21 | 84.0 | <0.001* |
| 11. | 4.5 | 3 | 12.0 | 22 | 88.0 | <0.001* |
| 12. | 4.5 | 0 | 0.0 | 25 | 100.0 | <0.001* |
| 13. | 4.5 | 4 | 16.0 | 21 | 84.0 | <0.001* |
| 14. | 4.5 | 7 | 28.0 | 18 | 72.0 | 0.028* |
| 15. | 4.5 | 3 | 12.0 | 22 | 88.0 | <0.001* |
| 16. | 4.5 | 7 | 28.0 | 18 | 72.0 | 0.028* |
| 17. | 4.5 | 9 | 36.0 | 16 | 64.0 | 0.162 |
| 18. | 4.5 | 5 | 20.0 | 20 | 80.0 | 0.003* |
| 19. | 4.5 | 6 | 24.0 | 19 | 76.0 | 0.009* |
| 20. | 4.5 | 6 | 24.0 | 19 | 76.0 | 0.009* |

*Significant

TABLE 29
SPQ Responses on Day 28 after daily use of Test Article 2

| | | Top Box | | | | |
|---|---|---|---|---|---|---|
| Test Article 2 | | Not Top Box | | Top Box | | |
| Question | Score for Top Box | N | Percent | N | Percent | p-value |
| 1. | 4.5 | 5 | 23.8 | 16 | 76.2 | 0.016* |
| 2. | 4.5 | 2 | 9.5 | 19 | 90.5 | <0.001* |
| 3. | 4.5 | 1 | 4.8 | 20 | 95.2 | <0.001* |
| 4. | 4.5 | 4 | 19.0 | 17 | 81.0 | 0.005* |
| 5. | 4.5 | 0 | 0.0 | 21 | 100.0 | <0.001* |
| 6. | 4.5 | 1 | 4.8 | 20 | 95.2 | <0.001* |
| 7. | 4.5 | 6 | 28.6 | 15 | 71.4 | 0.050* |
| 8. | 4.5 | 8 | 38.1 | 13 | 61.9 | 0.275 |
| 9. | 4.5 | 5 | 23.8 | 16 | 76.2 | 0.016* |
| 10. | 4.5 | 2 | 9.5 | 19 | 90.5 | <0.001* |
| 11. | 4.5 | 0 | 0.0 | 21 | 100.0 | <0.001* |
| 12. | 4.5 | 0 | 0.0 | 21 | 100.0 | <0.001* |
| 13. | 4.5 | 3 | 14.3 | 18 | 85.7 | 0.001* |
| 14. | 4.5 | 4 | 19.0 | 17 | 81.0 | 0.005* |
| 15. | 4.5 | 2 | 9.5 | 19 | 90.5 | <0.001* |
| 16. | 4.5 | 3 | 14.3 | 18 | 85.7 | 0.001* |
| 17. | 4.5 | 7 | 33.33 | 14 | 66.7 | 0.127 |
| 18. | 4.5 | 4 | 19.0 | 17 | 81.0 | 0.005* |
| 19. | 4.5 | 4 | 19.0 | 17 | 81.0 | 0.005* |
| 20. | 4.5 | 3 | 14.3 | 18 | 85.7 | 0.001* |

*Significant

TABLE 30
SPQ Responses on Day 28 after daily use of Test Article 3

| | | Top Box | | | | |
|---|---|---|---|---|---|---|
| Test Article 3 | | Not Top Box | | Top Box | | |
| Question | Score for Top Box | N | Percent | N | Percent | p-value |
| 1. | 4.5 | 5 | 23.8 | 16 | 76.2 | 0.016* |
| 2. | 4.5 | 1 | 4.8 | 20 | 95.2 | <0.001* |
| 3. | 4.5 | 1 | 4.8 | 20 | 95.2 | <0.001* |
| 4. | 4.5 | 4 | 19.0 | 17 | 81.0 | 0.005* |
| 5. | 4.5 | 1 | 4.8 | 20 | 95.2 | <0.001* |
| 6. | 4.5 | 2 | 9.5 | 19 | 90.5 | <0.001* |
| 7. | 4.5 | 6 | 28.6 | 15 | 71.4 | 0.050* |
| 8. | 4.5 | 7 | 33.3 | 14 | 66.7 | 0.127 |
| 9. | 4.5 | 5 | 23.8 | 16 | 76.2 | 0.016* |
| 10. | 4.5 | 1 | 4.8 | 20 | 95.2 | <0.001* |
| 11. | 4.5 | 2 | 9.5 | 19 | 90.5 | <0.001* |
| 12. | 4.5 | 0 | 0.0 | 21 | 100.0 | <0.001* |
| 13. | 4.5 | 0 | 0.0 | 21 | 100.0 | <0.001* |
| 14. | 4.5 | 3 | 14.3 | 18 | 85.7 | 0.001* |
| 15. | 4.5 | 1 | 4.8 | 20 | 95.2 | <0.001* |
| 16. | 4.5 | 2 | 9.5 | 19 | 90.5 | <0.001* |
| 17. | 4.5 | 4 | 19.0 | 17 | 81.0 | 0.005* |
| 18. | 4.5 | 4 | 19.0 | 17 | 81.0 | 0.005* |
| 19. | 4.5 | 4 | 19.0 | 17 | 81.0 | 0.005* |
| 20. | 4.5 | 3 | 14.3 | 18 | 85.7 | 0.001* |

*Significant

### Conclusion

Taken together, the data collected from this study demonstrates that cosmetic compositions containing CBG are effective at reducing erythema, induration, flaking, and scaling in psoriatic lesions.

Under the conditions of the study, all three treatment test articles significantly reduced erythema following 28 days of use (p<0.001). Test Article 3 effectuated the greatest reduction of severity following 28 days, achieving a percent change of 89.11%. Statistical analysis showed all three treatment products performed significantly at reducing erythema when compared to the vehicle product (“Placebo Article”) (p<0.001).

Additionally, all three treatment test articles significantly reduced induration following 28 days of use (p<0.001). Test Article 3 effectuated the greatest reduction of severity following 28 days, achieving a percent change of 92.25%. Statistical analysis showed all three treatment products performed significantly at reducing induration when compared to the vehicle product (“Placebo Article”) (p<0.001).

All three treatment test articles significantly reduced flaking following 28 days of use (p<0.001). Test Article 3 achieved the greatest reduction of severity following 28 days, achieving a percent change of 97.10%. Statistical analysis showed all three treatment products performed significantly at reducing flaking when compared to the vehicle product (“Placebo Article”) (p<0.001).

Moreover, all three treatment test articles significantly reduced scaling following 28 days of use (p<0.001). Test Article 3 effectuated the greatest reduction of severity following 28 days, achieving a percent change of 87.64%. Statistical analysis showed all three treatment products performed significantly at reducing flaking when compared to the vehicle product (“Placebo Article”) (p<0.001).

### Example 5. Evaluating the Ability of CBG to Improve Skin Appearance in Subjects Having Acne

### Summary of the Clinical Study

This example describes the results of a human clinical study that was conducted with the aim of investigating the ability of CBG to improve the outward appearance of skin in subjects having acne. Particularly, the objective of the study was to evaluate the efficacy of three test articles and a placebo on subjects with mild-to-moderate outbreaks and self-assessed troubled skin on the face. For the purposes of the study, mild acne is defined by the presence of greater than 5 inflammatory lesions and 10 or more non-inflammatory lesions, whereas moderate acne is defined by the presence of 10 or more inflammatory lesions and 10 or more non-inflammatory lesions (open and closed comedones on the face). Subjects were issued their corresponding test article (or placebo) to use twice daily: once in the morning and again in the evening. Efficacy was assessed using a multi-pronged approach that included clinical assessments, bioinstrumental measurements, and a subjective assessment.

Clinical assessments included the following: a count of inflammatory and non-inflammatory acne lesions and an assessment of global acne severity (using the IGA scale) at baseline, Day 7, Day 14, and Day 28.

Bioinstrument assessments included the following: a measurement of sebum with a SEBUMETER® SM815 at baseline, Day 7, Day 14, and Day 28; a subjective assessment by completion of subjective perception questionnaires on Day 17, Day 14, and Day 28; and clinical photography images of the test site taken at baseline, Day 7, Day 14, and Day 28.

The study had a duration of 4 weeks and enrolled approximately 90 health subjects, including males and females of ages 18-40 years old. All subjects had mild-to-moderate outbreaks and self-assessed troubled skin.

### Materials and Methods

### Study Design

This study was a single-blind, home-use trial. Subjects were issued their corresponding test article (or placebo) to use twice daily: once in the morning and again in the evening. This twice-daily regimen was continued throughout the duration of the 4-week test period. Study assessments were conducted according to the table below.

TABLE 31
Schedule of assessments

| | Study Time Point | | | |
|---|---|---|---|---|
| | Baseline | Day 7 | Day 14 | Day 28 |
| | Visit | | | |
| | 1 | 2 | 3 | 4 |
| Informed Consent | ✓ | | | |
| Inclusion/Exclusion Criteria | ✓ | | | |
| Assessment of Acne Lesions | ✓ | ✓ | ✓ | ✓ |
| (Count of lesions & IGA) | | | | |
| Subject self-assessment of | ✓ | ✓ | ✓ | ✓ |
| burning, stinging and itching, | | | | |
| dryness/scaling and swelling of | | | | |
| the face. | | | | |
| Assessment of redness, | ✓ | ✓ | ✓ | ✓ |
| dryness/scaling and swelling of | | | | |
| the face. | | | | |
| Sebumeter | ✓ | ✓ | ✓ | ✓ |
| Clinical Photography | ✓ | ✓ | ✓ | ✓ |
| Test Article Distribution (″D) | D | | | C |
| and Collection (“C”) | | | | |
| Self-Perception Questionnaire | | ✓ | ✓ | ✓ |
| Test Article Compliance Check | | ✓ | ✓ | ✓ |
| Adverse Event Review | | ✓ | ✓ | ✓ |

### Selection of Subjects

Subjects that participated in this study were enrolled on the basis of the following inclusion criteria and exclusion criteria.

### Inclusion Criteria:

Healthy, male and female volunteers, aged 18 years or older;
Self-assessed troubled (e.g. acne, blemishes) skin;
Fitzpatrick skin types I through V;
Mild (5-9 inflammatory lesions and 10 or more non-inflammatory lesions) to moderate (≥10 inflammatory lesions and 10 or more non-inflammatory lesions) acne on the body, excluding the face, as determined by the evaluator at the baseline visit;
If applicable, users of estrogens/birth control pills must have been on the treatment regimen for at least 3 months and be willing to continue use for study duration;
Females of child-bearing potential (FCBP): A female is considered not to be of childbearing potential if she is post-menopausal with at least 12 consecutive months of amenorrhea or has undergone surgical sterilization. While on the study, FCBP who engage in activity in which conception is possible must use one of the approved contraceptive options described below:
Option 1: Any one of the following methods: hormonal contraception (oral, injection, implant, transdermal patch, vaginal ring); intrauterine device (IUD); tubal ligation; or partner's vasectomy; OR Option 2: Male or female condom (latex condom or non latex condom not composed of natural membrane, plus one additional barrier method: (a) diaphragm with spermicide; (b) cervical cap with spermicide; or (c) contraceptive sponge with spermicide.
Willing to follow study instructions and available to attend the study visits; and
Willing to provide written informed consent and sign photography release.

### Exclusion Criteria:

Female subjects: Self-reported pregnant or breast feeding, or planning to become pregnant during the course of the study;
Known allergy or hypersensitivity to acne treatment products containing sulfur;
Current skin disease of any type in the test area (e.g. eczema, psoriasis, rosacea, seborrheic dermatitis, vitiligo, etc.), or under the treatment of a doctor for any skin condition;
Current severe cystic acne, acne conglobate, acne fulminans, or secondary acne (chloracne or drug-induced acne) in the test area;
Any conditions on the test site that would interfere with evaluations (i.e. tattoos, scars, open cuts, sunburn, piercings, excessive hair, etc.);
Use of topical treatments such as OTC (over the counter) acne medication topical anti-inflammatory medications, salicylic acid, corticosteroids, antibiotics, anti-bacterials, peroxide-containing products, or retinoids within 2 weeks of baseline;
Use of depigmenting medications such as hydroquinone during the 14 days prior to the study start;
Insulin dependent diabetes;
Concurrent medication that might affect the response to the test articles including routine use of anti-inflammatory medications, anti-histamines, and steroids;
History of Crohn's disease, or clinically significant enteritis (regional enteritis, ulcerative colitis, pseudomembranous colitis, antibiotic-associated colitis);
Microdermabrasion or laser treatment in the test area within six months of the study;
Medical condition which, in the opinion of the investigator, would compromise the safety of the subject or confound study results;
Use of systemic antibiotics, corticosteroids, antimalarials or oral dapsone within 4 weeks of baseline;
Use of other anti-acne medications, including isotretinoin or spironolactone, within 6 months of baseline;
Participation on an investigational drug study within 4 months of the baseline visit.

Additionally, subjects were prepared to accept certain prohibitions and restrictions. Particularly, subjects agreed to continue to use their usual skin cleansing products during the 4-weeks treatment period. Subjects also agreed to attend all study visits with a clean test area (e.g., no make-up or skincare products, such as moisturizer, sunscreen, etc.) Subjects agreed not to consume any hot or caffeinated foods or beverages or to smoke within one hour of study visits. Subjects agreed not to wet/wash the test area for at least 8 hours prior to the study visit. Subjects were willing to protect the test area from excessive sun exposure, tanning, and burning during the 4-week treatment period.

All subjects were informed of the nature, purpose, and known risks of the study both orally and in writing. Furthermore, all subjects were advised that they were free to withdraw from the study at any time without being obliged to give a reason. The study conformed to the requirements of the 1964 Declaration of Helsinki and its subsequent amendments (World Medical Association; 2013).

### Test Article and Placebo Compositions

The three test articles and the placebo article included the ingredients shown in Table 32.

TABLE 32
Article ingredients

| Article | Ingredients |
|---|---|
| Test Article 1 | Capric/Caprylic triglyceride and Cannabigerol |
| Test Article 2 | Squalane, Capric/Caprylic triglyceride, and Cannabigerol |
| Test Article 3 | Squalane, Capric/Caprylic Triglyceride, |
| | Cannabis Sativa Seed Oil, Cannabigerol, |
| | Cannabis Sativa Flower/Leaf/Stem Extract |
| | (Broad Spectrum), and Lavandula |
| | Angustifolia (lavender) Oil |
| Placebo Article | Capric/Caprylic triglyceride |

During the study, the test and placebo articles were applied in the form of droplets at the designated skin site. Test and placebo articles were applied after the subject cleansed the designated area.

### Study Procedure

At the inception of the study, subjects reported to the testing facility where informed consent was obtained and eligibility verified. Once accepted, subjects underwent baseline visual and clinical assessments. Following these assessments, subjects were given one of the 4 articles noted in Table 32, above. 10 subjects from each treatment group were then randomly selected to have clinical photography images taken of their faces at baseline. Subjects were given an information sheet with directions for usage, along with a diary sheet to complete each time they used the test or placebo article.

Subjects then returned to the study facility following 7 days of home use. Subjects underwent the same clinical and visual assessments as were conducted during the baseline visit. All subjects were given a self-perception questionnaire to complete (Table 33) on Day 7, Day 14, and Day 28 following daily administration of the test articles or the placebo article.

TABLE 33
Self-Perception Questionnaire

| | | | | | |
|---|---|---|---|---|---|
| Regarding the use of the test | Strongly | | Neither Agree | | Strongly |
| article | Disagree | Disagree | nor Disagree | Agree | Agree |

My skin appears clearer after using
this product.
I noticed a reduction in facial acne
or blemishes within “X” weeks of
using this product.
I noticed less breakouts after the
first “X” weeks using this product.
I noticed a reduction in redness
within “X” weeks of using this
product.
The acne scars on my face are
overall less visible after using this
product.
This product controls my breakout.
This product has reduced the
appearance of white heads.
This product has reduced the
appearance of black heads.
This product has reduced the size
of my pores.
This product has reduced the
appearance of pustules.
The product reduced the redness
of my skin.
This product has improved the
overall appearance of my skin.
This product has improved my
overall skin tone.
This product has made my skin
look more even.
This product helped brighten my
complexion.
I would continue to use this product
to help with my acne.
I would consider replacing my
usual products with this product.
I feel more confident and happier
with the use of this product.
I would recommend this product to
a friend or family member.
I am satisfied with the performance
of this product.

Clinical photography images were then taken of the same subjects that were photographed during the baseline visit.

Subjects then returned to the study facility following 14 days of home use, and again after 28 days of home use. Clinical and visual assessments and clinical photography were repeated during each visit in the manner conducted during the Day 7 visit.

### Assessments

A variety of assessments were conducted in this study, including grader evaluation, bioinstrumental measurement, and subjective perception. All assessments were made on the subject's test site that was identified during the baseline study visit.

Grader assessments included a count of acne lesions, global assessment of acne, a count of inflammatory and non-inflammatory acne lesions, and tactile skin texture evaluations. At each test facility, the same trained grader conducted the grader assessments.

Acne lesion counts included non-inflammatory lesions consisting of open and closed comedones and inflammatory lesions consisting of papules, pustules, and nodular lesions. Acne lesions were counted at baseline, Day 7, Day 14, and Day 28.

Global assessment of acne severity was conducted in accordance with the scale below.

TABLE 34
Scale of acne severity
Investigators Global Assessment (IGA) Scale

| Grade | Definition |
|---|---|
| 0 | Clear skin with no inflammatory or |
| | noninflammatory lesions |
| 1 | Almost clear; rare noninflammatory lesions with no |
| | more than one small inflammatory lesion |
| 2 | Mild severity; greater than Grade 1; some |
| | noninflammatory lesions with no more than |
| | a few inflammatory lesions (papules/pustules |
| | only, no nodular lesions) |
| 3 | Moderate severity; greater than Grade 2; up to many |
| | noninflammatory lesions and may have some inflammatory |
| | lesions, but no more than one small nodular lesion |
| 4 | Severe; greater than Grade 3; up to many |
| | noninflammatory and inflammatory lesions, |
| | but no more than a few nodular lesions |

Subjects were assigned a redness/during/scaling score using the scale below:

### Redness/Dryness/Scaling Grading Scale

0.0: No apparent cutaneous involvement.
0.5: Faint, barely perceptible erythema or slight dryness (glazed appearance).
1.0: Faint but definite erythema, no eruptions or broken skin or No erythema but definite dryness; may have epidermal fissuring.
1.5: Well-defined erythema or faint erythema with definite dryness, may have epidermal fissuring.
2.0: Moderate erythema, may have a very few papules or Deep fissures, moderate-to-severe erythema in the cracks.
2.5: Moderate erythema with barely perceptible oedema or severe erythema (halo effect around the edges), may have a few papules or moderate-to-severe erythema.
3.0: Severe erythema (beet redness), may have generalized papules or moderate-to-severe erythema with slight oedema (edges well defined by raising).
3.5: Moderate-to-severe erythema with moderate oedema or moderate-to-severe erythema with isolated eschar formations or vesicles.
4.0: Generalized vesicles or eschar formations or moderate-to-severe erythema and/or oedema.
   Subjects were assigned a swelling score using the scale below:

### Swelling Grading Scale

0 None
1 Very Slight (Barely Perceptible)
2 Slight edema (Edges of area well defined by definite raising)
3 Moderate edema (raised 1 mm)
4 Severe edema (raised >1 mm and extending beyond area of exposure).

### Bioinstrumental Measurements

Bioinstrument analyses were conducted using a Sebumeter, which measures the amount of sebum (oil) on the skin. Submeter readings were in units of μg of sebum per cm² of skin. Sebumeter measurements were taken at baseline, Day 7, Day 14, and Day 28.

### Clinical Photography

Clinical photography was utilized to capture high-resolution digital images of subjects' faces. The following equipment and settings were used to capture the images:
Camera: Nikon D5500
Lens: Nikon 18-140 mm
All Images shot on camera raw at ISO 200 1/125^{th} @ F20
Main light: 4X Soft box @135 W
Fill light Speed Lite—Camera Mounted
Background light Speed Lite—remote

### Statistical Analysis

Descriptive statistics (mean, standard deviation) were generated for all data. Multiple readings taken with the same instrument at a given time point were averaged for the purpose of statistical analysis.

Analyses for the clinical grading of global acne consisted of within-treatment assessments on changes from baseline utilizing Wilcoxon's Signed Rank Test. Percent change from baseline data were also determined. Three measures of mean percent in lesion counts were obtained: total inflammatory lesions (pustules and papules), total non-inflammatory lesions (open and closed comedones), and total lesions (sum of total inflammatory and non-inflammatory lesions). A between-treatment one-way ANOVA analysis was also conducted to examine differences between treatments and the placebo product in all variables.

Within-treatment analysis of bioinstrumentation data utilized t-tests.

Self-perception questionnaire responses were summarized specifying percent agreement for each response on Day 7, Day 14, and Day 28. Responses of “Strongly Agree” and “Agree” were counted as the top box scores.

All statistical tests of hypothesis employed a level of significance of 0.05 and no adjustments were made for the number of tests performed.

A formal sample size calculation was not required for this study.

### Results

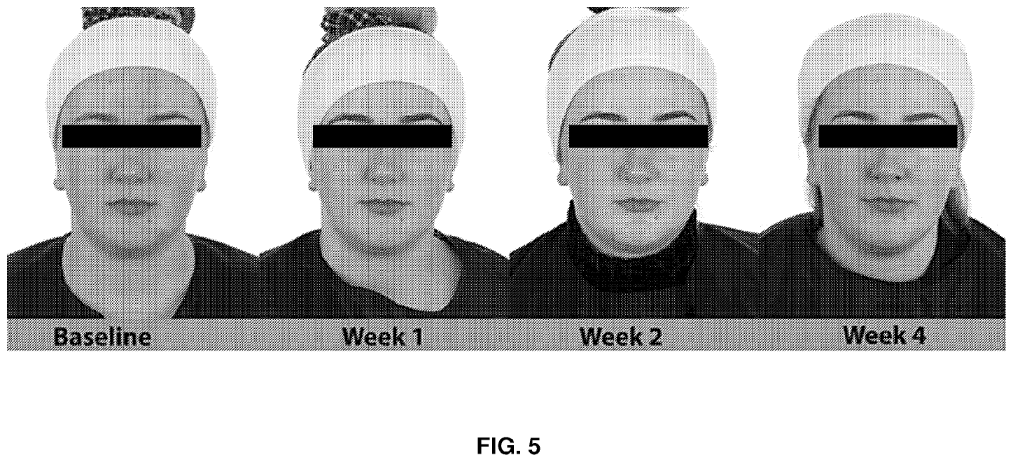

The results of this study demonstrate that cosmetic compositions containing CBG effectuate an improvement in the outward appearance of the skin in human subjects having acne. Particularly, as is shown in FIG. 5, Test Article 3 resulted in a visibly noticeable reduction in acne and clearing of the skin over the course of the 4-week study period.

Upon visual inspection, subjects treated with each test article showed improvements in the quantity of inflammatory and non-inflammatory acne lesions (Tables 35 and 36) following 28 consecutive days of use (p<0.001).

TABLE 35
Number of inflammatory and non-inflammatory acne lesions

| | | | | | Within- | Percent |
|---|---|---|---|---|---|---|
| | | | | Mean | Treatment | Change |
| | | | Treatment | Difference | Signed Rank | from |
| Attribute | Evaluation | N | Mean | From Baseline | p-value | Baseline |
| Placebo | Baseline | 21 | 8.7619 | | | |
| Article | | | | | | |
| | Day 7 | 21 | 10.0476 | −1.28571 | 1.000 | 14.67 |
| | Day 14 | 21 | 10.3333 | −1.57143 | 1.000 | 17.93 |
| | Day 28 | 21 | 11.4286 | −2.66667 | 0.130 | 30.44 |
| Test Article 1 | Baseline | 22 | 11.5000 | | | |
| | Day 7 | 22 | 9.6364 | 1.86364 | 1.000 | −16.21 |
| | Day 14 | 22 | 6.1818 | 5.31818 | 0.111 | −46.25 |
| | Day 28 | 22 | 4.7273 | 6.77273* | 0.018 | −58.89 |
| Test Article 2 | Baseline | 22 | 10.3182 | | | |
| | Day 7 | 22 | 5.9545 | 4.36364* | .000 | −42.29 |
| | Day 14 | 22 | 3.2500 | 7.06818* | .000 | −68.50 |
| | Day 28 | 22 | 1.2045 | 9.11364* | .000 | −88.33 |
| Test Article 3 | Baseline | 23 | 12.9130 | | | |
| | Day 7 | 23 | 6.6087 | 6.30435* | .003 | −48.82 |
| | Day 14 | 23 | 3.1087 | 9.80435* | .000 | −75.93 |
| | Day 28 | 23 | 0.8696 | 12.04348* | .000 | −93.27 |

*Significant change from baseline

TABLE 36
Difference in number of inflammatory
and non-inflammatory acne lesions
Test Article 1, Test Article 2, Test Article 3
vs Placebo Article (Between treatment analysis)

| Test article | Timepoint | p-value |
|---|---|---|
| Test Article 1 | Baseline | 1.000 |
| | Day 7 | 1.000 |
| | Day 14 | 0.005 |
| | Day 28 | 0.000 |
| Test Article 2 | Baseline | 1.000 |
| | Day 7 | 0.120 |
| | Day 14 | 0.000 |
| | Day 28 | 0.000 |
| Test Article 2 | Baseline | 0.400 |
| | Day 7 | 0.283 |
| | Day 14 | 0.000 |
| | Day 28 | 0.000 |

* Significant change compared to the placebo article at the given time point.

Upon visual inspection, subjects treated with each test article showed improvements in global acne severity using the IGA scale (Tables 37 and 38) following 28 consecutive days of use.

TABLE 37
Severity of acne lesions

| | | | | Mean | Within-Treatment | |
|---|---|---|---|---|---|---|
| | | | | Difference | Signed Rank | Percent |
| Attribute | Evaluation | N | Treatment Mean | From Baseline | p-value | Change Mean |
| Placebo Article | Baseline | 21 | 1.9286 | | | |
| | Day 7 | 21 | 1.8810 | 0.04762 | 1.000 | −2.47 |
| | Day 14 | 21 | 1.7619 | 0.16667 | 1.000 | −8.64 |
| | Day 28 | 21 | 2.1667 | −0.23810 | 1.000 | 12.35 |
| Test Article 1 | Baseline | 22 | 1.8864 | | | |
| | Day 7 | 22 | 1.6136 | 0.27273 | 0.375 | −14.46 |
| | Day 14 | 22 | 1.2500 | 0.63636* | <0.001* | −33.74 |
| | Day 28 | 22 | 1.1136 | 0.77273* | <0.001* | −40.97 |
| Test Article 2 | Baseline | 22 | 1.9773 | | | |
| | Day 7 | 22 | 1.3864 | 0.59091* | 0.001 | −29.88 |
| | Day 14 | 22 | 0.9318 | 1.04545* | 0.000 | −52.88 |
| | Day 28 | 22 | 0.5000 | 1.47727* | 0.000 | −74.71 |
| Test Article 3 | Baseline | 23 | 2.2391 | | | |
| | Day 7 | 23 | 1.4348 | .80435* | 0.000 | −35.92 |
| | Day 14 | 23 | 0.7609 | 1.47826* | 0.000 | −66.02 |
| | Day 28 | 23 | 0.4130 | 1.82609* | 0.000 | −81.56 |

*Significant change from baseline

TABLE 38
Difference in severity of acne lesions
Test Article 1, Test Article 2, Test Article 3 vs
Placebo Article (Between treatment analysis)

| Test article | Timepoint | p-value |
|---|---|---|
| Test Article 1 | Baseline | 1.000 |
| | Day 7 | 0.778 |
| | Day 14 | 0.002 |
| | Day 28 | 0.000 |
| Test Article 2 | Baseline | 1.000 |
| | Day 7 | 0.035 |
| | Day 14 | 0.000 |
| | Day 28 | 0.000 |
| Test Article 2 | Baseline | 0.727 |
| | Day 7 | 0.069 |
| | Day 14 | 0.000 |
| | Day 28 | 0.000 |

* Significant change compared to the placebo article at the given time point.

Upon visual inspection, subjects treated with each test article showed improvement in number of open comedones (Tables 39 and 40) following 28 consecutive days of use.

TABLE 39
Open comedones count

| | | | | | Within-Treatment | |
|---|---|---|---|---|---|---|
| | | | | Mean Difference | Signed Rank | Percent |
| Attribute | Evaluation | N | Treatment Mean | From Baseline | p-value | Change Mean |
| Placebo Article | Baseline | 21 | 22.4286 | | | |
| | Day 7 | 21 | 22.2857 | 0.14286 | 1.000 | −0.64 |
| | Day 14 | 21 | 22.3810 | 0.04762 | 1.000 | −0.21 |
| | Day 28 | 21 | 23.7143 | −1.28571 | 1.000 | 5.73 |
| Test Article 1 | Baseline | 22 | 19.3182 | | | |
| | Day 7 | 22 | 16.0909 | 3.22727 | 1.000 | −16.71 |
| | Day 14 | 22 | 12.5227 | 6.79545 | 1.000 | −35.18 |
| | Day 28 | 22 | 11.2273 | 8.09091 | 1.000 | −41.88 |
| Test Article 2 | Baseline | 22 | 10.1364 | | | |
| | Day 7 | 22 | 5.7727 | 4.36364* | 0.014 | −43.05 |
| | Day 14 | 22 | 2.8182 | 7.31818* | 0.000 | −72.20 |
| | Day 28 | 22 | 1.7273 | 8.40909* | 0.000 | −82.96 |
| Test Article 3 | Baseline | 23 | 9.7391 | | | |
| | Day 7 | 23 | 4.8696 | 4.86957* | 0.000 | −50.00 |
| | Day 14 | 23 | 2.0870 | 7.65217* | 0.000 | −78.57 |
| | Day 28 | 23 | .9130 | 8.82609* | 0.000 | −90.63 |

*Significant change from baseline

TABLE 40
Difference in open comedones count
Test Article 1, Test Article 2, Test Article 3 vs
Placebo Article (Between treatment analysis)

| Test article | Timepoint | p-value |
|---|---|---|
| Test Article 1 | Baseline | 1.000 |
| | Day 7 | 0.585 |
| | Day 14 | 0.050 |
| | Day 28 | 0.007 |
| Test Article 2 | Baseline | 0.011 |
| | Day 7 | 0.000 |
| | Day 14 | 0.000 |
| | Day 28 | 0.000 |
| Test Article 2 | Baseline | 0.007 |
| | Day 7 | 0.000 |
| | Day 14 | 0.000 |
| | Day 28 | 0.000 |

* Significant change compared to the placebo article at the given time point.

Upon visual inspection, subjects treated with each test article showed improvements in number of closed comedones (Tables 41 and 42) following 28 consecutive days of use.

TABLE 41
Closed comedones count

| | | | | | Within-Treatment | Percent |
|---|---|---|---|---|---|---|
| | | | | Mean Difference | Signed Rank | Change |
| Attribute | Evaluation | N | Treatment Mean | From Baseline | p-value | Mean |
| Placebo Article | Baseline | 21 | 6.1429 | | | |
| | Day 7 | 21 | 6.1429 | 0.00000 | 1.000 | 0.00 |
| | Day 14 | 21 | 6.0476 | 0.09524 | 1.000 | −1.55 |
| | Day 28 | 21 | 6.1429 | 0.00000 | 1.000 | 0.00 |
| Test Article 1 | Baseline | 22 | 6.1364 | | | |
| | Day 7 | 22 | 4.6818 | 1.45455 | 0.526 | −23.70 |
| | Day 14 | 22 | 3.0909 | 3.04545* | 0.003 | −49.63 |
| | Day 28 | 22 | 2.4091 | 3.72727* | 0.000 | −60.74 |
| Test Article 2 | Baseline | 22 | 6.0000 | | | |
| | Day 7 | 22 | 3.5000 | 2.50000* | 0.013 | −41.67 |
| | Day 14 | 22 | 1.7727 | 4.22727* | 0.000 | −70.46 |
| | Day 28 | 22 | 1.0455 | 4.95455* | 0.000 | −82.58 |
| Test Article 3 | Baseline | 23 | 5.5652 | | | |
| | Day 7 | 23 | 2.6957 | 2.86957* | 0.001 | −51.56 |
| | Day 14 | 23 | 1.2609 | 4.30435* | 0.000 | −77.34 |
| | Day 28 | 23 | 0.5652 | 5.00000* | 0.000 | −89.84 |

*Significant change from baseline

TABLE 42
Difference in closed comedones count
Test Article 1, Test Article 2, Test Article 3 vs
Placebo Article (Between treatment analysis)

| Test article | Timepoint | p-value |
|---|---|---|
| Test Article 1 | Baseline | 1.000 |
| | Day 7 | 0.547 |
| | Day 14 | 0.001 |
| | Day 28 | 0.000 |
| Test Article 2 | Baseline | 1.000 |
| | Day 7 | 0.016 |
| | Day 14 | 0.000 |
| | Day 28 | 0.000 |
| Test Article 2 | Baseline | 1.000 |
| | Day 7 | 0.001 |
| | Day 14 | 0.000 |
| | Day 28 | 0.000 |

* Significant change compared to the placebo article at the given time point.

Upon visual inspection, subjects treated with each test article showed improvements in number of papules (Tables 43 and 44) following 28 consecutive days of use.

TABLE 43
Number of papules

| | | | | Mean | Within- | |
|---|---|---|---|---|---|---|
| | | | | Difference | Treatment | Percent |
| | | | Treatment | From | Signed Rank | Change |
| Attribute | Evaluation | N | Mean | Baseline | p-value | Mean |
| Placebo Article | Baseline | 21 | 1.5238 | | | |
| | Day 7 | 21 | 1.2857 | 0.23810 | 1.000 | −15.63 |
| | Day 14 | 21 | 1.0000 | 0.52381 | 0.522 | −34.37 |
| | Day 28 | 21 | 1.1905 | 0.33333 | 1.000 | −21.87 |
| Test Article 1 | Baseline | 22 | 1.4091 | | | |
| | Day 7 | 22 | 0.4545 | 0.95455* | 0.001 | −67.75 |
| | Day 14 | 22 | 0.1364 | 1.27273* | 0.000 | −90.32 |
| | Day 28 | 22 | 0.2273 | 1.18182* | 0.000 | −83.87 |
| Test Article 2 | Baseline | 22 | 1.0000 | | | |
| | Day 7 | 22 | 0.4091 | 0.59091 | 0.163 | −59.09 |
| | Day 14 | 22 | 0.2273 | 0.77273* | 0.025 | −77.27 |
| | Day 28 | 22 | 0.1364 | 0.86364* | 0.009 | −86.36 |
| Test Article 3 | Baseline | 23 | 1.2609 | | | |
| | Day 7 | 23 | 0.3043 | 0.95652* | 0.000 | −77.87 |
| | Day 14 | 23 | 0.0000 | 1.26087* | 0.000 | −100.00 |
| | Day 28 | 23 | 0.0000 | 1.26087* | 0.000 | −100.00 |

*Significant change from baseline

TABLE 44
Difference in papules count
Test Article 1, Test Article 2, Test Article 3 vs
Placebo Article (Between treatment analysis)

| Test article | Timepoint | p-value |
|---|---|---|
| Test Article 1 | Baseline | 1.000 |
| | Day 7 | 0.006 |
| | Day 14 | 0.000 |
| | Day 28 | 0.000 |
| Test Article 2 | Baseline | 1.000 |
| | Day 7 | 0.003 |
| | Day 14 | 0.000 |
| | Day 28 | 0.000 |
| Test Article 2 | Baseline | 1.000 |
| | Day 7 | 0.001 |
| | Day 14 | 0.000 |
| | Day 28 | 0.000 |

* Significant change compared to the placebo article at the given time point.

Upon visual inspection, subjects treated with each test article showed improvements in number of pustules (Tables 45 and 46) following 28 consecutive days of use.

TABLE 45
Pustules count

| | | | | Mean | Within- | |
|---|---|---|---|---|---|---|
| | | | | Difference | Treatment | Percent |
| | | | Treatment | From | Signed Rank | Change |
| Attribute | Evaluation | N | Mean | Baseline | p-value | Mean |
| Placebo Article | Baseline | 21 | 1.5952 | | | |
| | Day 7 | 21 | 1.3571 | 0.23810 | 1.000 | −14.93 |
| | Day 14 | 21 | 1.1905 | 0.40476 | 0.638 | −25.37 |
| | Day 28 | 21 | 1.0476 | 0.54762 | 0.180 | −34.33 |
| Test Article 1 | Baseline | 22 | 0.4091 | | | |
| | Day 7 | 22 | 0.0909 | 0.31818 | 0.161 | −77.78 |
| | Day 14 | 22 | 0.0000 | 0.40909* | 0.029 | −100.00 |
| | Day 28 | 22 | 0.1818 | 0.22727 | 0.667 | −55.56 |
| Test Article 2 | Baseline | 22 | 0.7727 | | | |
| | Day 7 | 22 | 0.1818 | 0.59091 | 0.122 | −76.47 |
| | Day 14 | 22 | 0.1364 | 0.63636 | 0.076 | −82.35 |
| | Day 28 | 22 | 0.0000 | 0.77273* | 0.016 | −100.00 |
| Test Article 3 | Baseline | 23 | 0.9130 | | | |
| | Day 7 | 23 | 0.1304 | 0.78261 | 0.000 | −85.72 |
| | Day 14 | 23 | 0.0000 | 0.91304* | 0.000 | −100.00 |
| | Day 28 | 23 | 0.0000 | 0.91304* | 0.000 | −100.00 |

*Significant change from baseline

TABLE 46
Difference in pustules count
Test Article 1, Test Article 2, Test Article 3 vs
Placebo Article (Between treatment analysis)

| Test article | Timepoint | p-value |
|---|---|---|
| Test Article 1 | Baseline | 0.002 |
| | Day 7 | 0.000 |
| | Day 14 | 0.000 |
| | Day 28 | 0.000 |
| Test Article 2 | Baseline | 0.070 |
| | Day 7 | 0.000 |
| | Day 14 | 0.000 |
| | Day 28 | 0.000 |
| Test Article 2 | Baseline | 0.201 |
| | Day 7 | 0.000 |
| | Day 14 | 0.000 |
| | Day 28 | 0.000 |

* Significant change compared to the placebo article at the given time point.

Upon visual inspection, subjects treated with each test article showed improvement in redness severity (Tables 47 and 48) following 28 consecutive days of use.

TABLE 47
Redness severity

| | | | | Mean | Within- | |
|---|---|---|---|---|---|---|
| | | | | Difference | Treatment | Percent |
| | | | Treatment | From | Signed Rank | Change |
| Attribute | Evaluation | N | Mean | Baseline | p-value | Mean |
| Placebo Article | Baseline | 21 | 1.3810 | | | |
| | Day 7 | 21 | 1.2143 | .16667 | 1.000 | −12.07 |
| | Day 14 | 21 | .8333 | .54762 | 0.069 | −39.66 |
| | Day 28 | 21 | .3333 | 1.04762* | <0.001* | −75.87 |
| Test Article 1 | Baseline | 22 | .5909 | | | |
| | Day 7 | 22 | .1591 | .43182* | .020 | −73.07 |
| | Day 14 | 22 | .0455 | .54545* | .002 | −92.30 |
| | Day 28 | 22 | .0455 | .54545* | .002 | −92.30 |
| Test Article 2 | Baseline | 22 | .9773 | | | |
| | Day 7 | 22 | .6364 | .34091 | 1.000 | −34.88 |
| | Day 14 | 22 | .3182 | .65909 | .061 | −67.44 |
| | Day 28 | 22 | .0909 | .88636* | .004 | −90.70 |
| Test Article 3 | Baseline | 23 | 1.1087 | | | |
| | Day 7 | 23 | .6087 | .08696 | .262 | −45.10 |
| | Day 14 | 23 | .3261 | .08696 | .262 | −70.59 |
| | Day 28 | 23 | .0435 | .08696 | .262 | −96.08 |

*Significant change from baseline

TABLE 48
Difference in redness severity
Test Article 1, Test Article 2, Test Article 3
vs Placebo Article (Between treatment analysis)

| Test article | Timepoint | p-value |
|---|---|---|
| Test Article 1 | Baseline | 0.100 |
| | Day 7 | 0.000 |
| | Day 14 | 0.000 |
| | Day 28 | 0.012 |
| Test Article 2 | Baseline | 1.000 |
| | Day 7 | 0.130 |
| | Day 14 | 0.015 |
| | Day 28 | 0.053 |
| Test Article 2 | Baseline | 1.000 |
| | Day 7 | 0.091 |
| | Day 14 | 0.016 |
| | Day 28 | 0.010 |

* Significant change compared to the placebo article at the given time point.

Upon visual inspection, subjects treated with each test article showed improvements in dryness severity (Tables 49 and 50) following 28 consecutive days of use.

TABLE 49
Dryness severity

| | | | | Mean | Within- | |
|---|---|---|---|---|---|---|
| | | | | Difference | Treatment | Percent |
| | | | Treatment | From | Signed Rank | Change |
| Attribute | Evaluation | N | Mean | Baseline | p-value | Mean |
| Placebo Article | Baseline | 21 | .1905 | | | |
| | Day 7 | 21 | .1905 | .00000 | 1.000 | 0.00 |
| | Day 14 | 21 | .0952 | .09524 | 1.000 | −50.03 |
| | Day 28 | 21 | .1429 | .04762 | 1.000 | −24.99 |
| Test Article 1 | Baseline | 22 | .0909 | | | |
| | Day 7 | 22 | .0000 | .09091 | .261 | −100.0 |
| | Day 14 | 22 | .0000 | .09091 | .261 | −100.0 |
| | Day 28 | 22 | .0000 | .09091 | .261 | −100.0 |
| Test Article 2 | Baseline | 22 | .0000 | | | |
| | Day 7 | 22 | .0000 | 0.000 | 1.000 | 0.00 |
| | Day 14 | 22 | .0000 | 0.000 | 1.000 | 0.00 |
| | Day 28 | 22 | .0000 | 0.000 | 1.000 | 0.00 |
| Test Article 3 | Baseline | 23 | .0870 | | | |
| | Day 7 | 23 | .0000 | 0.000 | 1.000 | 0.00 |
| | Day 14 | 23 | .0000 | 0.000 | 1.000 | 0.00 |
| | Day 28 | 23 | .0000 | 0.000 | 1.000 | 0.00 |

* Significant change from baseline

TABLE 50
Difference in dryness severity
Test Article 1, Test Article 2, Test Article 3
vs Placebo Article (Between treatment analysis)

| Test article | Timepoint | p-value |
|---|---|---|
| Test Article 1 | Baseline | 1.000 |
| | Day 7 | 0.012 |
| | Day 14 | 0.218 |
| | Day 28 | 0.288 |
| Test Article 2 | Baseline | 0.192 |
| | Day 7 | 0.012 |
| | Day 14 | 0.218 |
| | Day 28 | 0.288 |
| Test Article 2 | Baseline | 1.000 |
| | Day 7 | 0.011 |
| | Day 14 | 0.207 |
| | Day 28 | 0.274 |

* Significant change compared to the placebo article at the given time point.

Upon visual inspection, subjects treated with each test article showed improvements in scaling severity (Tables 51 and 52) following 28 consecutive days of use.

TABLE 51
Scaling severity

| | | | | Mean | Within- | |
|---|---|---|---|---|---|---|
| | | | | Difference | Treatment | Percent |
| | | | Treatment | From | Signed Rank | Change |
| Attribute | Evaluation | N | Mean | Baseline | p-value | Mean |
| Placebo Article | Baseline | 21 | 0.0000 | | | |
| | Day 7 | 21 | 0.0000 | 0.000 | 1.000 | 0.00 |
| | Day 14 | 21 | 0.0000 | 0.000 | 1.000 | 0.00 |
| | Day 28 | 21 | 0.0000 | 0.000 | 1.000 | 0.00 |
| Test Article 1 | Baseline | 22 | 0.0000 | | | |
| | Day 7 | 22 | 0.0000 | 0.000 | 1.000 | 0.00 |
| | Day 14 | 22 | 0.0000 | 0.000 | 1.000 | 0.00 |
| | Day 28 | 22 | 0.0000 | 0.000 | 1.000 | 0.00 |
| Test Article 2 | Baseline | 22 | 0.0000 | | | |
| | Day 7 | 22 | 0.0000 | 0.000 | 1.000 | 0.00 |
| | Day 14 | 22 | 0.0000 | 0.000 | 1.000 | 0.00 |
| | Day 28 | 22 | 0.0000 | 0.000 | 1.000 | 0.00 |
| Test Article 3 | Baseline | 23 | 0.0000 | | | |
| | Day 7 | 23 | 0.0000 | 0.000 | 1.000 | 0.00 |
| | Day 14 | 23 | 0.0000 | 0.000 | 1.000 | 0.00 |
| | Day 28 | 23 | 0.0000 | 0.000 | 1.000 | 0.00 |

* Significant change from baseline

TABLE 52
Difference in scaling severity
Test Article 1, Test Article 2, Test Article 3
vs Placebo Article (Between treatment analysis)

| Test article | Timepoint | p-value |
|---|---|---|
| Test Article 1 | Baseline | 1.000 |
| | Day 7 | 1.000 |
| | Day 14 | 1.000 |
| | Day 28 | 1.000 |
| Test Article 2 | Baseline | 1.000 |
| | Day 7 | 1.000 |
| | Day 14 | 1.000 |
| | Day 28 | 1.000 |
| Test Article 2 | Baseline | 1.000 |
| | Day 7 | 1.000 |
| | Day 14 | 0.207 |
| | Day 28 | 0.274 |

* Significant change compared to the placebo article at the given time point.

Upon visual inspection, subjects treated with each test article showed improvements in swelling severity (Tables 53 and 54) following 28 consecutive days of use.

TABLE 53
Swelling severity

| | | | | Mean | Within- | |
|---|---|---|---|---|---|---|
| | | | | Difference | Treatment | Percent |
| | | | Treatment | From | Signed Rank | Change |
| Attribute | Evaluation | N | Mean | Baseline | p-value | Mean |
| Placebo Article | Baseline | 21 | 0.0000 | | | |
| | Day 7 | 21 | 0.0000 | 0.000 | 1.000 | 0.00 |
| | Day 14 | 21 | 0.0000 | 0.000 | 1.000 | 0.00 |
| | Day 28 | 21 | 0.0000 | 0.000 | 1.000 | 0.00 |
| Test Article 1 | Baseline | 22 | 0.0000 | | | |
| | Day 7 | 22 | 0.0000 | 0.000 | 1.000 | 0.00 |
| | Day 14 | 22 | 0.0000 | 0.000 | 1.000 | 0.00 |
| | Day 28 | 22 | 0.0000 | 0.000 | 1.000 | 0.00 |
| Test Article 2 | Baseline | 22 | 0.0000 | | | |
| | Day 7 | 22 | 0.0000 | 0.000 | 1.000 | 0.00 |
| | Day 14 | 22 | 0.0000 | 0.000 | 1.000 | 0.00 |
| | Day 28 | 22 | 0.0000 | 0.000 | 1.000 | 0.00 |
| Test Article 3 | Baseline | 23 | 0.0000 | | | |
| | Day 7 | 23 | 0.0000 | 0.000 | 1.000 | 0.00 |
| | Day 14 | 23 | 0.0000 | 0.000 | 1.000 | 0.00 |
| | Day 28 | 23 | 0.0000 | 0.000 | 1.000 | 0.00 |

* Significant change from baseline

TABLE 54
Difference in swelling severity
Test Article 1, Test Article 2, Test Article 3
vs Placebo Article (Between treatment analysis)

| Test article | Timepoint | p-value |
|---|---|---|
| Test Article 1 | Baseline | 1.000 |
| | Day 7 | 1.000 |
| | Day 14 | 1.000 |
| | Day 28 | 1.000 |
| Test Article 2 | Baseline | 1.000 |
| | Day 7 | 1.000 |
| | Day 14 | 1.000 |
| | Day 28 | 1.000 |
| Test Article 2 | Baseline | 1.000 |
| | Day 7 | 1.000 |
| | Day 14 | 1.000 |
| | Day 28 | 1.000 |

* Significant change compared to the placebo article at the given time point.

Upon visual inspection, subjects treated with each test article showed improvements in amount of sebum (Tables 55 and 56) following 28 consecutive days of use.

TABLE 55
Measured sebum (μg/cm²)

| | | | | Mean | Within- | |
|---|---|---|---|---|---|---|
| | | | | Difference | Treatment | Percent |
| | | | Treatment | From | Signed Rank | Change |
| Attribute | Evaluation | N | Mean | Baseline | p-value | Mean |
| Placebo Article | Baseline | 21 | 73.6238 | | | |
| | Day 7 | 21 | 73.6095 | 0.01429 | 1.000 | −0.02 |
| | Day 14 | 21 | 73.6333 | −0.00952 | 1.000 | 0.01 |
| | Day 28 | 21 | 73.6524 | −0.02857 | 1.000 | 0.04 |
| Test Article 1 | Baseline | 22 | 74.5864 | | | |
| | Day 7 | 22 | 72.6818 | 1.90455 | 0.124 | −2.55 |
| | Day 14 | 22 | 69.7318 | 4.85455* | 0.000 | −6.51 |
| | Day 28 | 22 | 68.1773 | 6.40909* | 0.000 | −8.59 |
| Test Article 2 | Baseline | 22 | 74.1591 | | | |
| | Day 7 | 22 | 71.4455 | 2.71364* | 0.035 | −3.66 |
| | Day 14 | 22 | 68.0591 | 6.10000* | 0.000 | −8.23 |
| | Day 28 | 22 | 58.7409 | 15.41818* | 0.000 | −20.79 |
| Test Article 3 | Baseline | 23 | 74.3609 | | | |
| | Day 7 | 23 | 70.4348 | 3.92609* | 0.000 | −5.28 |
| | Day 14 | 23 | 66.9826 | 7.37826* | 0.000 | −9.92 |
| | Day 28 | 23 | 54.7087 | 19.65217* | 0.000 | −26.43 |

*Significant change from baseline

TABLE 56
Difference in measured sebum
Test Article 1, Test Article 2, Test Article 3
vs Placebo Article (Between treatment analysis)

| Test article | Timepoint | p-value |
|---|---|---|
| Test Article 1 | Baseline | 1.000 |
| | Day 7 | 1.000 |
| | Day 14 | 0.001 |
| | Day 28 | 0.000 |
| Test Article 2 | Baseline | 1.000 |
| | Day 7 | 0.172 |
| | Day 14 | 0.000 |
| | Day 28 | 0.000 |
| Test Article 2 | Baseline | 1.000 |
| | Day 7 | 0.008 |
| | Day 14 | 0.000 |
| | Day 28 | 0.000 |

* Significant change compared to the placebo article at the given time point.

### Self-Perception of Acne Severity

A self-perception questionnaire (SPQ), as shown in Table 33, was also provided to each group (Test Article 1, Test Article 2, Test Article 3, and Placebo Article groups) to assess the severity of the acne lesions at Day 7, Day 14, and Day 28.

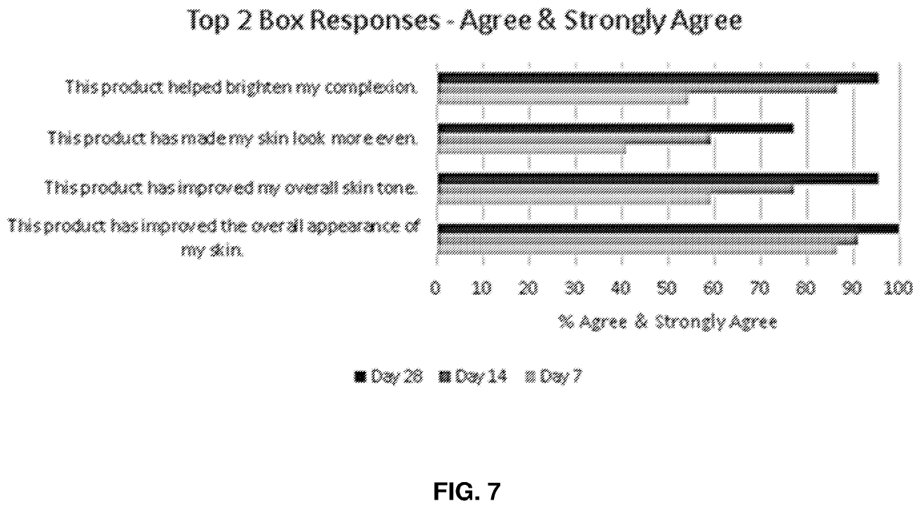

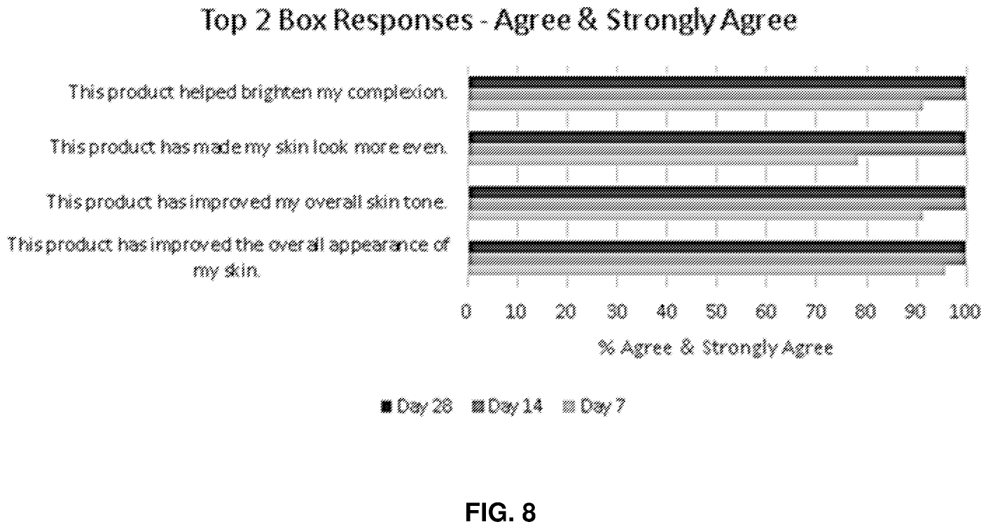

SPQ responses are summarized in Tables 57-68. SPQ responses to questions pertaining to skin brightening effects of Test Article 2 (FIG. 7) and Test Article 3 (FIG. 8) were graphed at Day 7, Day 14, and Day 28 of the study. Responses of “Strongly Agree” and “Agree” were considered “top box” scores.

TABLE 57
SPQ Responses on Day 7 after daily use of Placebo Article

| | | Top Box | | | | |
|---|---|---|---|---|---|---|
| Placebo Article | | Not | | | | |
| | Score for | Top Box | | Top Box | | |
| Question | Top Box | N | Percent | N | Percent | p-value |
| 1. My skin appears clearer after using this product. | 4, 5 | 11 | 52.4 | 10 | 47.6 | 0.827 |
| 2. I noticed a reduction in facial acne or blemishes | 4, 5 | 15 | 71.4 | 6 | 28.6 | 0.050* |
| within “1” week of using this product. | | | | | | |
| 3. I noticed less breakouts after the first “1” week | 4, 5 | 16 | 76.2 | 5 | 23.8 | 0.016* |
| using this product. | | | | | | |
| 4. I noticed a reduction in redness within “1” week of | 4, 5 | 16 | 76.2 | 5 | 23.8 | 0.016* |
| using this product. | | | | | | |
| 5. The acne scars on my face are overall less | 4, 5 | 20 | 95.2 | 1 | 4.8 | <0.001* |
| visible after using this product. | | | | | | |
| 6. This product controls my breakout. | 4, 5 | 20 | 95.2 | 1 | 4.8 | <0.001* |
| 7. This product has reduced the appearance of | 4, 5 | 14 | 66.7 | 7 | 33.3 | 0.127 |
| white heads. | | | | | | |
| 8. This product has reduced the appearance of | 4, 5 | 15 | 71.4 | 6 | 28.6 | 0.050* |
| black heads. | | | | | | |
| 9. This product has reduced the size of my pores. | 4, 5 | 15 | 71.4 | 6 | 28.6 | 0.050* |
| 10. This product has reduced the appearance of | 4, 5 | 18 | 85.7 | 3 | 14.3 | 0.001* |
| pustules. | | | | | | |
| 11. The product reduced the redness of my skin. | 4, 5 | 17 | 81.0 | 4 | 19.0 | 0.005* |
| 12. This product has improved the overall | 4, 5 | 15 | 71.4 | 6 | 28.6 | 0.050* |
| appearance of my skin. | | | | | | |
| 13. This product has improved my overall skin tone. | 4, 5 | 19 | 90.5 | 2 | 9.5 | <0.001* |
| 14. This product has made my skin look more even. | 4, 5 | 19 | 90.5 | 2 | 9.5 | <0.001* |
| 15. This product helped brighten my complexion. | 4, 5 | 18 | 85.7 | 3 | 14.3 | 0.001* |
| 16. I would continue to use this product to help with | 4, 5 | 17 | 81.0 | 4 | 19.0 | 0.005* |
| my acne. | | | | | | |
| 17. I would consider replacing my usual products | 4, 5 | 19 | 90.5 | 2 | 9.5 | <0.001* |
| with this product. | | | | | | |
| 18. I feel more confident and happier with the use of | 4, 5 | 17 | 81.0 | 4 | 19.0 | 0.005* |
| this product. | | | | | | |
| 19. I would recommend this product to a friend or | 4, 5 | 17 | 81.0 | 4 | 19.0 | 0.005* |
| family member. | | | | | | |
| 20. I am satisfied with the performance of this | 4, 5 | 17 | 81.0 | 4 | 19.0 | 0.005* |
| product. | | | | | | |

*Significant

TABLE 58
SPQ Responses on Day 7 after daily use of Article 1

| | | Top Box | | | | |
|---|---|---|---|---|---|---|
| Test Article 1 | | Not | | | | |
| | Score for | Top Box | | Top Box | | |
| Question | Top Box | N | Percent | N | Percent | p-value |
| 1. My skin appears clearer after using this product. | 4, 5 | 5 | 22.7 | 17 | 77.3 | 0.011* |
| 2. I noticed a reduction in facial acne or blemishes | 4, 5 | 6 | 27.3 | 16 | 72.7 | 0.033* |
| within “1” week of using this product. | | | | | | |
| 3. I noticed less breakouts after the first “1” week | 4, 5 | 9 | 40.8 | 13 | 59.1 | 0.394 |
| using this product. | | | | | | |
| 4. I noticed a reduction in redness within “1” week of | 4, 5 | 8 | 36.4 | 14 | 63.6 | 0.201 |
| using this product. | | | | | | |
| 5. The acne scars on my face are overall less | 4, 5 | 15 | 68.2 | 7 | 31.8 | 0.088 |
| visible after using this product. | | | | | | |
| 6. This product controls my breakout. | 4, 5 | 12 | 54.5 | 10 | 45.5 | 0.670 |
| 7. This product has reduced the appearance of | 4, 5 | 6 | 27.3 | 16 | 72.7 | 0.033* |
| white heads. | | | | | | |
| 8. This product has reduced the appearance of | 4, 5 | 10 | 45.5 | 12 | 54.5 | 0.670 |
| black heads. | | | | | | |
| 9. This product has reduced the size of my pores. | 4, 5 | 12 | 54.5 | 10 | 45.5 | 0.670 |
| 10. This product has reduced the appearance of | 4, 5 | 12 | 54.5 | 10 | 45.5 | 0.670 |
| pustules. | | | | | | |
| 11. The product reduced the redness of my skin. | 4, 5 | 7 | 31.8 | 15 | 68.2 | 0.088 |
| 12. This product has improved the overall | 4, 5 | 4 | 18.2 | 18 | 81.8 | 0.003* |
| appearance of my skin. | | | | | | |
| 13. This product has improved my overall skin tone. | 4, 5 | 10 | 45.5 | 12 | 54.5 | 0.670 |
| 14. This product has made my skin look more even. | 4, 5 | 11 | 50.0 | 11 | 50.0 | 1.000 |
| 15. This product helped brighten my complexion. | 4, 5 | 10 | 45.5 | 12 | 54.5 | 0.670 |
| 16. I would continue to use this product to help with | 4, 5 | 6 | 27.3 | 16 | 72.7 | 0.033* |
| my acne. | | | | | | |
| 17. I would consider replacing my usual products | 4, 5 | 12 | 54.5 | 10 | 45.5 | 0.670 |
| with this product. | | | | | | |
| 18. I feel more confident and happier with the use of | 4, 5 | 13 | 59.1 | 9 | 40.9 | 0.394 |
| this product. | | | | | | |
| 19. I would recommend this product to a friend or | 4, 5 | 8 | 36.4 | 14 | 63.6 | 0.201 |
| family member. | | | | | | |
| 20. I am satisfied with the performance of this | 4, 5 | 7 | 31.8 | 15 | 68.2 | 0.088 |
| product. | | | | | | |

*Significant

TABLE 59
SPQ Responses on Day 7 after daily use of Article 2

| | | Top Box | | | | |
|---|---|---|---|---|---|---|
| Test Article 2 | | Not | | | | |
| | Score for | Top Box | | Top Box | | |
| Question | Top Box | N | Percent | N | Percent | p-value |
| 1. My skin appears clearer after using this product. | 4, 5 | 3 | 13.6 | 19 | 86.4 | <0.001* |
| 2. I noticed a reduction in facial acne or blemishes | 4, 5 | 5 | 22.7 | 17 | 77.3 | 0.011* |
| within “1” week of using this product. | | | | | | |
| 3. I noticed less breakouts after the first “1” week | 4, 5 | 8 | 36.4 | 14 | 63.6 | 0.201 |
| using this product. | | | | | | |
| 4. I noticed a reduction in redness within “1” week of | 4, 5 | 5 | 22.7 | 17 | 77.3 | 0.011* |
| using this product. | | | | | | |
| 5. The acne scars on my face are overall less | 4, 5 | 13 | 59.1 | 9 | 40.9 | 0.394 |
| visible after using this product. | | | | | | |
| 6. This product controls my breakout. | 4, 5 | 10 | 45.5 | 12 | 54.5 | 0.670 |
| 7. This product has reduced the appearance of | 4, 5 | 4 | 18.2 | 18 | 81.8 | 0.003 |
| white heads. | | | | | | |
| 8. This product has reduced the appearance of | 4, 5 | 12 | 54.5 | 10 | 45.5 | 0.670 |
| black heads. | | | | | | |
| 9. This product has reduced the size of my pores. | 4, 5 | 11 | 50.0 | 11 | 50.0 | 1.000 |
| 10. This product has reduced the appearance of | 4, 5 | 9 | 40.9 | 13 | 59.1 | 0.394 |
| pustules. | | | | | | |
| 11. The product reduced the redness of my skin. | 4, 5 | 5 | 22.7 | 17 | 77.3 | 0.011* |
| 12. This product has improved the overall | 4, 5 | 3 | 13.6 | 19 | 86.4 | <0.001* |
| appearance of my skin. | | | | | | |
| 13. This product has improved my overall skin tone. | 4, 5 | 9 | 40.9 | 13 | 59.1 | 0.394 |
| 14. This product has made my skin look more even. | 4, 5 | 13 | 59.1 | 9 | 40.9 | 0.394 |
| 15. This product helped brighten my complexion. | 4, 5 | 10 | 45.5 | 12 | 54.5 | 0.394 |
| 16. I would continue to use this product to help with | 4, 5 | 5 | 22.7 | 17 | 77.3 | 0.011* |
| my acne. | | | | | | |
| 17. I would consider replacing my usual products | 4, 5 | 9 | 40.9 | 13 | 59.1 | 0.394 |
| with this product. | | | | | | |
| 18. I feel more confident and happier with the use of | 4, 5 | 9 | 40.9 | 13 | 59.1 | 0.394 |
| this product. | | | | | | |
| 19. I would recommend this product to a friend or | 4, 5 | 3 | 13.6 | 19 | 86.4 | <0.001* |
| family member. | | | | | | |
| 20. I am satisfied with the performance of this | 4, 5 | 5 | 22.7 | 17 | 77.3 | 0.011* |
| product. | | | | | | |

*Significant

TABLE 60
SPQ Responses on Day 7 after daily use of Article 3

| | | Top Box | | | | |
|---|---|---|---|---|---|---|
| Test Article 3 | | Not | | | | |
| | Score for | Top Box | | Top Box | | |
| Question | Top Box | N | Percent | N | Percent | p-value |
| 1. My skin appears clearer after using this product. | 4, 5 | 1 | 4.3 | 22 | 95.7 | <0.001* |
| 2. I noticed a reduction in facial acne or blemishes | 4, 5 | 2 | 8.7 | 21 | 91.3 | <0.001* |
| within “1” week of using this product. | | | | | | |
| 3. I noticed less breakouts after the first “1” week | 4, 5 | 4 | 17.4 | 19 | 82.6 | 0.002* |
| using this product. | | | | | | |
| 4. I noticed a reduction in redness within “1” week of | 4, 5 | 2 | 8.7 | 21 | 91.3 | <0.001* |
| using this product. | | | | | | |
| 5. The acne scars on my face are overall less | 4, 5 | 8 | 34.8 | 15 | 65.2 | 0.144 |
| visible after using this product. | | | | | | |
| 6. This product controls my breakout. | 4, 5 | 3 | 13.0 | 20 | 87.0 | <0.001* |
| 7. This product has reduced the appearance of | 4, 5 | 3 | 13.0 | 20 | 87.0 | <0.001* |
| white heads. | | | | | | |
| 8. This product has reduced the appearance of | 4, 5 | 4 | 17.4 | 19 | 82.6 | 0.002* |
| black heads. | | | | | | |
| 9. This product has reduced the size of my pores. | 4, 5 | 4 | 17.4 | 19 | 82.6 | 0.002* |
| 10. This product has reduced the appearance of | 4, 5 | 3 | 13.0 | 20 | 87.0 | <0.001* |
| pustules. | | | | | | |
| 11. The product reduced the redness of my skin. | 4, 5 | 2 | 8.7 | 21 | 91.3 | <0.001* |
| 12. This product has improved the overall | 4, 5 | 1 | 4.3 | 22 | 95.7 | <0.001* |
| appearance of my skin. | | | | | | |
| 13. This product has improved my overall skin tone. | 4, 5 | 2 | 8.7 | 21 | 91.3 | <0.001* |
| 14. This product has made my skin look more even. | 4, 5 | 5 | 21.7 | 18 | 78.3 | 0.007* |
| 15. This product helped brighten my complexion. | 4, 5 | 2 | 8.7 | 21 | 91.3 | <0.001* |
| 16. I would continue to use this product to help with | 4, 5 | 2 | 8.7 | 21 | 91.3 | <0.001* |
| my acne. | | | | | | |
| 17. I would consider replacing my usual products | 4, 5 | 3 | 13.0 | 20 | 87.0 | <0.001* |
| with this product. | | | | | | |
| 18. I feel more confident and happier with the use of | 4, 5 | 3 | 13.0 | 20 | 87.0 | <0.001* |
| this product. | | | | | | |
| 19. I would recommend this product to a friend or | 4, 5 | 2 | 8.7 | 21 | 91.3 | <0.001* |
| family member. | | | | | | |
| 20. I am satisfied with the performance of this | 4, 5 | 2 | 8.7 | 21 | 91.3 | <0.001* |
| product. | | | | | | |

*Significant

TABLE 61
SPQ Responses on Day 14 after daily use of Placebo Article

| | | Top Box | | | | |
|---|---|---|---|---|---|---|
| Placebo Article | | Not | | | | |
| | Score for | Top Box | | Top Box | | |
| Question | Top Box | N | Percent | N | Percent | p-value |
| 1. My skin appears clearer after using this product. | 4, 5 | 12 | 57.1 | 9 | 42.9 | 0.513 |
| 2. I noticed a reduction in facial acne or blemishes | 4, 5 | 15 | 71.4 | 6 | 28.6 | 0.050* |
| within “2” weeks of using this product. | | | | | | |
| 3. I noticed less breakouts after the first “2” weeks | 4, 5 | 18 | 85.7 | 3 | 14.3 | 0.001* |
| using this product. | | | | | | |
| 4. I noticed a reduction in redness within “2” weeks of | 4, 5 | 16 | 76.2 | 5 | 23.8 | 0.016* |
| using this product. | | | | | | |
| 5. The acne scars on my face are overall less | 4, 5 | 21 | 100.0 | 0 | 0.0 | <0.001* |
| visible after using this product. | | | | | | |
| 6. This product controls my breakout. | 4, 5 | 20 | 95.2 | 1 | 4.8 | <0.001* |
| 7. This product has reduced the appearance of | 4, 5 | 14 | 66.7 | 7 | 33.3 | 0.127 |
| white heads. | | | | | | |
| 8. This product has reduced the appearance of | 4, 5 | 14 | 66.7 | 7 | 33.3 | 0.127 |
| black heads. | | | | | | |
| 9. This product has reduced the size of my pores. | 4, 5 | 17 | 81.0 | 4 | 19.0 | 0.005* |
| 10. This product has reduced the appearance of | 4, 5 | 20 | 95.2 | 1 | 4.8 | <0.001* |
| pustules. | | | | | | |
| 11. The product reduced the redness of my skin. | 4, 5 | 17 | 81.0 | 4 | 19.0 | 0.005* |
| 12. This product has improved the overall | 4, 5 | 17 | 81.0 | 4 | 19.0 | 0.005* |
| appearance of my skin. | | | | | | |
| 13. This product has improved my overall skin tone. | 4, 5 | 18 | 85.7 | 3 | 14.3 | 0.001* |
| 14. This product has made my skin look more even. | 4, 5 | 20 | 95.2 | 1 | 4.8 | <0.001* |
| 15. This product helped brighten my complexion. | 4, 5 | 18 | 85.7 | 3 | 14.3 | 0.001* |
| 16. I would continue to use this product to help with | 4, 5 | 19 | 90.5 | 2 | 9.5 | <0.001* |
| my acne. | | | | | | |
| 17. I would consider replacing my usual products | 4, 5 | 20 | 95.2 | 1 | 4.8 | <0.001* |
| with this product. | | | | | | |
| 18. I feel more confident and happier with the use of | 4, 5 | 18 | 85.7 | 3 | 14.3 | 0.001* |
| this product. | | | | | | |
| 19. I would recommend this product to a friend or | 4, 5 | 19 | 90.5 | 2 | 9.5 | <0.001* |
| family member. | | | | | | |
| 20. I am satisfied with the performance of this | 4, 5 | 19 | 90.5 | 2 | 9.5 | <0.001* |
| product. | | | | | | |

*Significant

TABLE 62
SPQ Responses on Day 14 after daily use of Article 1

| | | Top Box | | | | |
|---|---|---|---|---|---|---|
| Test Article 1 | | Not | | | | |
| | Score for | Top Box | | Top Box | | |
| Question | Top Box | N | Percent | N | Percent | p-value |
| 1. My skin appears clearer after using this product. | 4, 5 | 2 | 9.1 | 20 | 90.9 | <0.001* |
| 2. I noticed a reduction in facial acne or blemishes | 4, 5 | 2 | 9.1 | 20 | 90.9 | <0.001* |
| within “2” weeks of using this product. | | | | | | |
| 3. I noticed less breakouts after the first “2” weeks | 4, 5 | 4 | 18.2 | 18 | 81.8 | 0.003* |
| using this product. | | | | | | |
| 4. I noticed a reduction in redness within “2” weeks of | 4, 5 | 4 | 18.2 | 18 | 81.8 | 0.003* |
| using this product. | | | | | | |
| 5. The acne scars on my face are overall less | 4, 5 | 13 | 59.1 | 9 | 40.9 | 0.394 |
| visible after using this product. | | | | | | |
| 6. This product controls my breakout. | 4, 5 | 6 | 27.3 | 16 | 72.7 | 0.033* |
| 7. This product has reduced the appearance of | 4, 5 | 2 | 9.1 | 20 | 90.9 | <0.001* |
| white heads. | | | | | | |
| 8. This product has reduced the appearance of | 4, 5 | 4 | 18.2 | 18 | 81.8 | 0.003* |
| black heads. | | | | | | |
| 9. This product has reduced the size of my pores. | 4, 5 | 3 | 13.6 | 19 | 86.4 | <0.001* |
| 10. This product has reduced the appearance of | 4, 5 | 8 | 36.4 | 14 | 63.6 | 0.201 |
| pustules. | | | | | | |
| 11. The product reduced the redness of my skin. | 4, 5 | 4 | 18.2 | 18 | 81.8 | 0.003* |
| 12. This product has improved the overall | 4, 5 | 3 | 13.6 | 19 | 86.4 | <0.001* |
| appearance of my skin. | | | | | | |
| 13. This product has improved my overall skin tone. | 4, 5 | 4 | 18.2 | 18 | 81.8 | 0.003* |
| 14. This product has made my skin look more even. | 4, 5 | 7 | 31.8 | 15 | 68/2 | 0.088 |
| 15. This product helped brighten my complexion. | 4, 5 | 8 | 36.4 | 14 | 63.6 | 0.201 |
| 16. I would continue to use this product to help with | 4, 5 | 3 | 13.6 | 19 | 86.4 | <0.001* |
| my acne. | | | | | | |
| 17. I would consider replacing my usual products | 4, 5 | 9 | 40.9 | 13 | 59.1 | 0.394 |
| with this product. | | | | | | |
| 18. I feel more confident and happier with the use of | 4, 5 | 8 | 36.4 | 14 | 63.6 | 0.201 |
| this product. | | | | | | |
| 19. I would recommend this product to a friend or | 4, 5 | 3 | 13.6 | 19 | 86.4 | <0.001* |
| family member. | | | | | | |
| 20. I am satisfied with the performance of this | 4, 5 | 3 | 13.6 | 19 | 86.4 | <0.001* |
| product. | | | | | | |

*Significant

TABLE 63
SPQ Responses on Day 14 after daily use of Article 2

| | | Top Box | | | | |
|---|---|---|---|---|---|---|
| Test Article 2 | | Not | | | | |
| | Score for | Top Box | | Top Box | | |
| Question | Top Box | N | Percent | N | Percent | p-value |
| 1. My skin appears clearer after using this product. | 4, 5 | 0 | 0.0 | 22 | 100.0 | <0.001* |
| 2. I noticed a reduction in facial acne or blemishes | 4, 5 | 4 | 18.2 | 18 | 81.8 | 0.003* |
| within “2” weeks of using this product. | | | | | | |
| 3. I noticed less breakouts after the first “2” weeks | 4, 5 | 4 | 18.2 | 18 | 81.8 | 0.003* |
| using this product. | | | | | | |
| 4. I noticed a reduction in redness within “2” weeks of | 4, 5 | 1 | 4.5 | 21 | 95.5 | <0.001* |
| using this product. | | | | | | |
| 5. The acne scars on my face are overall less | 4, 5 | 10 | 45.5 | 12 | 54.5 | 0.670 |
| visible after using this product. | | | | | | |
| 6. This product controls my breakout. | 4, 5 | 3 | 13.6 | 19 | 86.4 | <0.001* |
| 7. This product has reduced the appearance of | 4, 5 | 1 | 4.5 | 21 | 95.5 | <0.001* |
| white heads. | | | | | | |
| 8. This product has reduced the appearance of | 4, 5 | 7 | 31.8 | 15 | 68.2 | 0.088 |
| black heads. | | | | | | |
| 9. This product has reduced the size of my pores. | 4, 5 | 7 | 31.8 | 15 | 68.2 | 0.088 |
| 10. This product has reduced the appearance of | 4, 5 | 5 | 22.7 | 17 | 77.3 | 0.011* |
| pustules. | | | | | | |
| 11. The product reduced the redness of my skin. | 4, 5 | 1 | 4.5 | 21 | 95.5 | <0.001* |
| 12. This product has improved the overall | 4, 5 | 2 | 9.1 | 20 | 90.9 | <0.001* |
| appearance of my skin. | | | | | | |
| 13. This product has improved my overall skin tone. | 4, 5 | 5 | 22.7 | 17 | 77.3 | 0.011* |
| 14. This product has made my skin look more even. | 4, 5 | 9 | 40.9 | 13 | 59.1 | 0.394 |
| 15. This product helped brighten my complexion. | 4, 5 | 3 | 13.6 | 19 | 86.4 | <0.001* |
| 16. I would continue to use this product to help with | 4, 5 | 4 | 18.2 | 18 | 81.8 | 0.003* |
| my acne. | | | | | | |
| 17. I would consider replacing my usual products | 4, 5 | 5 | 22.7 | 17 | 77.3 | 0.011* |
| with this product. | | | | | | |
| 18. I feel more confident and happier with the use of | 4, 5 | 6 | 27.3 | 16 | 72.7 | 0.033* |
| this product. | | | | | | |
| 19. I would recommend this product to a friend or | 4, 5 | 1 | 4.5 | 21 | 95.5 | <0.001* |
| family member. | | | | | | |
| 20. I am satisfied with the performance of this | 4, 5 | 2 | 9.1 | 20 | 90.9 | <0.001* |
| product. | | | | | | |

*Significant

TABLE 64
SPQ Responses on Day 14 after daily use of Article 3

| | | Top Box | | | | |
|---|---|---|---|---|---|---|
| Test Article 3 | | Not | | | | |
| | Score for | Top Box | | Top Box | | |
| Question | Top Box | N | Percent | N | Percent | p-value |
| 1. My skin appears clearer after using this product. | 4, 5 | 0 | 0.0 | 23 | 100.0 | <0.001* |
| 2. I noticed a reduction in facial acne or blemishes | 4, 5 | 1 | 4.3 | 22 | 95.7 | <0.001* |
| within “2” weeks of using this product. | | | | | | |
| 3. I noticed less breakouts after the first “2” weeks | 4, 5 | 0 | 0.0 | 23 | 100.0 | <0.001* |
| using this product. | | | | | | |
| 4. I noticed a reduction in redness within “2” weeks of | 4, 5 | 0 | 0.0 | 23 | 100.0 | <0.001* |
| using this product. | | | | | | |
| 5. The acne scars on my face are overall less | 4, 5 | 2 | 8.7 | 21 | 91.3 | <0.001* |
| visible after using this product. | | | | | | |
| 6. This product controls my breakout. | 4, 5 | 0 | 0.0 | 23 | 100.0 | <0.001* |
| 7. This product has reduced the appearance of | 4, 5 | 1 | 4.3 | 22 | 95.7 | <0.001* |
| white heads. | | | | | | |
| 8. This product has reduced the appearance of | 4, 5 | 3 | 13.0 | 20 | 87.0 | <0.001* |
| black heads. | | | | | | |
| 9. This product has reduced the size of my pores. | 4, 5 | 1 | 4.3 | 22 | 95.7 | <0.001* |
| 10. This product has reduced the appearance of | 4, 5 | 0 | 0.0 | 23 | 100.0 | <0.001* |
| pustules. | | | | | | |
| 11. The product reduced the redness of my skin. | 4, 5 | 0 | 0.0 | 23 | 100.0 | <0.001* |
| 12. This product has improved the overall | 4, 5 | 0 | 0.0 | 23 | 100.0 | <0.001* |
| appearance of my skin. | | | | | | |
| 13. This product has improved my overall skin tone. | 4, 5 | 0 | 0.0 | 23 | 100.0 | <0.001* |
| 14. This product has made my skin look more even. | 4, 5 | O | 0.0 | 23 | 100.0 | <0.001* |
| 15. This product helped brighten my complexion. | 4, 5 | O | 0.0 | 23 | 100.0 | <0.001* |
| 16. I would continue to use this product to help with | 4, 5 | 0 | 0.0 | 23 | 100.0 | <0.001* |
| my acne. | | | | | | |
| 17. I would consider replacing my usual products | 4, 5 | 0 | 0.0 | 23 | 100.0 | <0.001* |
| with this product. | | | | | | |
| 18. I feel more confident and happier with the use of | 4, 5 | 1 | 4.3 | 22 | 95.7 | <0.001* |
| this product. | | | | | | |
| 19. I would recommend this product to a friend or | 4, 5 | 0 | 0.0 | 23 | 100.0 | <0.001* |
| family member. | | | | | | |
| 20. I am satisfied with the performance of this | 4, 5 | 0 | 0.0 | 23 | 100.0 | <0.001* |
| product. | | | | | | |

*Significant

TABLE 65
SPQ Responses on Day 28 after daily use of Placebo Article

Top Box

| | |
|---|---|
| Placebo Article | Not Top |

| | | |
|---|---|---|
| Score for | Box | Top Box |

| Question | Top Box | N | Percent | N | Percent | p-value |
|---|---|---|---|---|---|---|
| 1. My skin appears clearer after using this product. | 4.5 | 11 | 52.4 | 10 | 47.6 | 0.827 |
| 2. I noticed a reduction in facial acne or blemishes | 4.5 | 15 | 71.4 | 6 | 28.6 | 0.050* |
| within “4” weeks of using this product. | | | | | | |
| 3. I noticed less breakouts after the first “4” weeks | 4.5 | 19 | 90.5 | 2 | 9.5 | <0.001* |
| using this product. | | | | | | |
| 4. I noticed a reduction in redness within “4” weeks | 4.5 | 14 | 66.7 | 7 | 33.3 | 0.127 |
| of using this product. | | | | | | |
| 5. The acne scars on my face are overall less | 4.5 | 20 | 95.2 | 1 | 4.8 | <0.001 |
| visible after using this product. | | | | | | |
| 6. This product controls my breakout. | 4.5 | 19 | 90.5 | 2 | 9.5 | <0.001* |
| 7. This product has reduced the appearance of | 4.5 | 14 | 66.7 | 7 | 33.3 | 0.127 |
| white heads. | | | | | | |
| 8. This product has reduced the appearance of | 4.5 | 15 | 71.4 | 6 | 28.6 | 0.050* |
| black heads. | | | | | | |
| 9. This product has reduced the size of my pores. | 4.5 | 18 | 85.7 | 3 | 14.3 | 0.001* |
| 10. This product has reduced the appearance of | 4.5 | 20 | 95.2 | 1 | 4.8 | <0.001* |
| pustules. | | | | | | |
| 11. The product reduced the redness of my skin. | 4.5 | 14 | 66.7 | 7 | 33.3 | 0.127 |
| 12. This product has improved the overall | 4.5 | 17 | 81.0 | 4 | 19.0 | 0.005* |
| appearance of my skin. | | | | | | |
| 13. This product has improved my overall skin tone. | 4.5 | 19 | 90.5 | 2 | 9.5 | <0.001* |
| 14. This product has made my skin look more even. | 4.5 | 20 | 95.2 | 1 | 4.8 | <0.001 |
| 15. This product helped brighten my complexion. | 4.5 | 18 | 85.7 | 3 | 14.3 | <0.001* |
| 16. I would continue to use this product to help with | 4.5 | 19 | 90.5 | 2 | 9.5 | <0.001* |
| my acne. | | | | | | |
| 17. I would consider replacing my usual products | 4.5 | 20 | 95.2 | 1 | 4.8 | <0.001* |
| with this product. | | | | | | |
| 18. I feel more confident and happier with the use of | 4.5 | 18 | 85.7 | 3 | 14.3 | <0.001* |
| this product. | | | | | | |
| 19. I would recommend this product to a friend or | 4.5 | 19 | 90.5 | 2 | 9.5 | <0.001* |
| family member. | | | | | | |
| 20. I am satisfied with the performance of this | 4.5 | 19 | 90.5 | 2 | 9.5 | <0.001* |
| product. | | | | | | |

*Significant

TABLE 66
SPQ Responses on Day 28 after daily use of Article 1

Top Box

| | |
|---|---|
| Test Article 1 | Not Top |

| | | |
|---|---|---|
| Score for | Box | Top Box |

| Question | Top Box | N | Percent | N | Percent | p-value |
|---|---|---|---|---|---|---|
| 1. My skin appears clearer after using this product. | 4.5 | 1 | 4.5 | 21 | 95.5 | <0.001* |
| 2. I noticed a reduction in facial acne or blemishes | 4.5 | 2 | 9.1 | 20 | 90.9 | <0.001* |
| within “4” weeks of using this product. | | | | | | |
| 3. I noticed less breakouts after the first “4” weeks | 4.5 | 4 | 18.2 | 18 | 81.8 | 0.003* |
| using this product. | | | | | | |
| 4. I noticed a reduction in redness within “4” weeks | 4.5 | 2 | 9.1 | 20 | 90.9 | <0.001* |
| of using this product. | | | | | | |
| 5. The acne scars on my face are overall less | 4.5 | 10 | 45.5 | 12 | 54.5 | 0.670 |
| visible after using this product. | | | | | | |
| 6. This product controls my breakout. | 4.5 | 4 | 18.2 | 18 | 81.8 | 0.003* |
| 7. This product has reduced the appearance of | 4.5 | 2 | 9.1 | 20 | 90.9 | <0.001* |
| white heads. | | | | | | |
| 8. This product has reduced the appearance of | 4.5 | 1 | 4.521 | | 95.5 | <0.001* |
| black heads. | | | | | | |
| 9. This product has reduced the size of my pores. | 4.5 | 3 | 13.6 | 19 | 86.4 | <0.001 |
| 10. This product has reduced the appearance of | 4.5 | 6 | 27.3 | 16 | 72.7 | 0.033 |
| pustules. | | | | | | |
| 11. The product reduced the redness of my skin. | 4.5 | 2 | 9.1 | 20 | 90.9 | <0.001 |
| 12. This product has improved the overall | 4.5 | 2 | 9.1 | 20 | 90.9 | <0.001* |
| appearance of my skin. | | | | | | |
| 13. This product has improved my overall skin tone. | 4.5 | 2 | 9.1 | 20 | 90.9 | <0.001* |
| 14. This product has made my skin look more even. | 4.5 | 7 | 31.8 | 15 | 68.2 | 0.088 |
| 15. This product helped brighten my complexion. | 4.5 | 6 | 27.3 | 16 | 72.7 | 0.033* |
| 16. I would continue to use this product to help with | 4.5 | 3 | 13.6 | 19 | 86.4 | <0.001* |
| my acne. | | | | | | |
| 17. I would consider replacing my usual products | 4.5 | 7 | 31.8 | 15 | 68.2 | 0.088 |
| with this product. | | | | | | |
| 18. I feel more confident and happier with the use of | 4.5 | 4 | 18.2 | 18 | 81.8 | 0.003* |
| this product. | | | | | | |
| 19. I would recommend this product to a friend or | 4.5 | 2 | 9.1 | 20 | 90.9 | <0.001* |
| family member. | | | | | | |
| 20. I am satisfied with the performance of this | 4.5 | 2 | 9.1 | 20 | 90.9 | <0.001* |
| product. | | | | | | |

*Significant

TABLE 67
SPQ Responses on Day 28 after daily use of Article 2

| | | Top Box | | | | |
|---|---|---|---|---|---|---|
| Test Article 2 | | Not Top | | | | |
| | Score for | Box | | Top Box | | |
| Question | Top Box | N | Percent | N | Percent | p-value |
| 1. My skin appears clearer after using this product. | 4.5 | 0 | 0.0 | 22 | 100.0 | <0.001* |
| 2. I noticed a reduction in facial acne or blemishes | 4.5 | 2 | 9.1 | 20 | 90.9 | <0.001* |
| within “4” weeks of using this product. | | | | | | |
| 3. I noticed less breakouts after the first “4” weeks | 4.5 | 2 | 9.1 | 20 | 90.9 | <0.001 |
| using this product. | | | | | | |
| 4. I noticed a reduction in redness within “4” weeks | 4.5 | 0 | 0.0 | 22 | 100.0 | <0.001* |
| of using this product. | | | | | | |
| 5. The acne scars on my face are overall less visible | 4.5 | 6 | 27.3 | 16 | 72.7 | 0.033* |
| after using this product. | | | | | | |
| 6. This product controls my breakout. | 4.5 | 2 | 9.1 | 20 | 90.9 | <0.001* |
| 7. This product has reduced the appearance of | 4.5 | 0 | 0.0 | 22 | 100.0 | <0.001* |
| white heads. | | | | | | |
| 8. This product has reduced the appearance of | 4.5 | 4 | 182 | 18 | 81.9 | 0.003* |
| black heads. | | | | | | |
| 9. This product has reduced the size of my pores. | 4.5 | 4 | 18.2 | 18 | 81.8 | 0.003* |
| 10. This product has reduced the appearance of | 4.5 | 4 | 18.2 | 18 | 81.8 | 0.003* |
| pustules. | | | | | | |
| 11. The product reduced the redness of my skin. | 4.5 | 0 | 0.0 | 22 | 100.0 | <0.001* |
| 12. This product has improved the overall | 4.5 | 0 | 0.0 | 22 | 100.0 | <0.001* |
| appearance of my skin. | | | | | | |
| 13. This product has improved my overall skin tone. | 4.5 | 1 | 4.5 | 21 | 95.5 | <0.001* |
| 14. This product has made my skin look more even. | 4.5 | 5 | 22.7 | 17 | 77.3 | 0.011* |
| 15. This product helped brighten my complexion. | 4.5 | 1 | 4.5 | 21 | 95.5 | <0.001* |
| 16. I would continue to use this product to help with | 4.5 | 1 | 4.5 | 21 | 95.5 | <0.001* |
| my acne. | | | | | | |
| 17. I would consider replacing my usual products | 4.5 | 2 | 9.1 | 20 | 90.9 | <0.001* |
| with this product. | | | | | | |
| 18. I feel more confident and happier with the use of | 4.5 | 2 | 9.1 | 20 | 90.9 | <0.001* |
| this product. | | | | | | |
| 19. I would recommend this product to a friend or | 4.5 | 0 | 0.0 | 22 | 100.0 | <0.001* |
| family member. | | | | | | |
| 20. I am satisfied with the performance of this | 4.5 | 1 | 4.5 | 21 | 95.5 | <0.001* |
| product. | | | | | | |

*Significant

TABLE 68
SPQ Responses on Day 28 after daily use of Article 3

| | | Top Box | | | | |
|---|---|---|---|---|---|---|
| Test Article 3 | | Not Top | | | | |
| | Score for | Box | | Top Box | | |
| Question | Top Box | N | Percent | N | Percent | p-value |
| 1. My skin appears clearer after using this product. | 4.5 | 0 | 0.0 | 23 | 100.0 | <0.001* |
| 2. I noticed a reduction in facial acne or blemishes | 4.5 | 0 | 0.0 | 23 | 100.0 | <0.001* |
| within “4” weeks of using this product. | | | | | | |
| 3. I noticed less breakouts after the first “4” weeks | 4.5 | 0 | 0.0 | 23 | 100.0 | <0.001* |
| using this product. | | | | | | |
| 4. I noticed a reduction in redness within “4” weeks | 4.5 | 0 | 0.0 | 23 | 100.0 | <0.001* |
| of using this product. | | | | | | |
| 5. The acne scars on my face are overall less visible | 4.5 | 1 | 4.3 | 22 | 95.7 | <0.001* |
| after using this product. | | | | | | |
| 6. This product controls my breakout. | 4.5 | 0 | 0.0 | 23 | 100.0 | <0.001* |
| 7. This product has reduced the appearance of | 4.5 | 0 | 0.0 | 23 | 100.0 | <0.001* |
| white heads. | | | | | | |
| 8. This product has reduced the appearance of | 4.5 | 2 | 8.7 | 21 | 91.3 | <0.001* |
| black heads. | | | | | | |
| 9. This product has reduced the size of my pores. | 4.5 | 0 | 0.0 | 23 | 100.0 | <0.001* |
| 10. This product has reduced the appearance of | 4.5 | 0 | 0.0 | 23 | 100.0 | <0.001* |
| pustules. | | | | | | |
| 11. The product reduced the redness of my skin. | 4.5 | 0 | 0.0 | 23 | 100.0 | <0.001* |
| 12. This product has improved the overall | 4.5 | 0 | 0.0 | 23 | 100.0 | <0.001* |
| appearance of my skin. | | | | | | |
| 13. This product has improved my overall skin tone. | 4.5 | 0 | 0.0 | 23 | 100.0 | <0.001* |
| 14. This product has made my skin look more even. | 4.5 | 0 | 0.0 | 23 | 100.0 | <0.001* |
| 15. This product helped brighten my complexion. | 4.5 | 0 | 0.0 | 23 | 100.0 | <0.001* |
| 16. I would continue to use this product to help with | 4.5 | 0 | 0.0 | 23 | 100.0 | <0.001* |
| my acne. | | | | | | |
| 17. I would consider replacing my usual products | 4.5 | 0 | 0.0 | 23 | 100.0 | <0.001* |
| with this product. | | | | | | |
| 18. I feel more confident and happier with the use of | 4.5 | 1 | 4.3 | 22 | 95.7 | <0.001* |
| this product. | | | | | | |
| 19. I would recommend this product to a friend or | 4.5 | 0 | 0.023 | | 100.0 | <0.001* |
| family member. | | | | | | |
| 20. I am satisfied with the performance of this | 4.5 | 0 | 0.0 | 23 | 100.0 | <0.001* |
| product. | | | | | | |

*Significant

### Conclusion

Taken together, the data collected from this study demonstrate that cosmetic compositions containing CBG are effective in reducing acne and improving the outward appearance of the skin.

Under the conditions of the study, all test articles significantly reduced skin sebum amounts, as measured by SEBUMETER®, by day 14 (p<0.001) when compared to baseline scores. Test Article 2 and Test Article 3 achieved significant reductions by day 7 when compared to baseline. Test Article 3 achieved the greatest reduction of 26.43% following 28 days. There were no significant differences between all treatments versus Placebo Article at baseline. Following baseline, all treatment sites showed significant reductions in sebum amount on the skin when compared to the placebo product following 28 days of use.

Additionally, all test articles significantly reduced acne lesion count grades by day 28 (p<0.05). Test Article 2 and Test Article 3 significantly reduced acne lesions by day 7 (p<0.005). Test Article 3 showed the greatest reduction of 93.27% following 28 days. There were no significant differences between all sites at the baseline visit. Following baseline, all treatment products showed significant differences at day 14 when compared to the Placebo Article (p<0.005).

All treatment products significantly reduced Global Acne grades by day 14 (p<0.001). Test Article 2 and Test Article 3 significantly reduced acne lesions by day 7 (p<0.005). Test article 3 showed the greatest reduction of 81.56% following 28 days. There were no significant differences between all sites at the baseline visit. Following baseline, all treatment products showed significant differences at day 14 when compared to the Placebo Article (p<0.005).

Test Article 2 and Test Article 3 showed improvement in self-assessed skin brightening and complexion (FIG. 7 and FIG. 8) with Test Article 3 showing significant improvements in skin brightening and complexion as early as day 7 (FIG. 8) of the study.

### Example 6. Evaluating the Ability of Cannabigerol to Improve Eczema

This example describes results from a human study that was conducted with the aim of assessing the ability of CBG to reduce skin irritation, itchiness, inflammation, and sensitivity in a subject having an eczema lesion.

### Materials and Methods

This study was performed by administering Test Article 3 from Examples 4 and 5, above, to a subject diagnosed as having eczema. Test Article 3 was administered to the subject at the site of the lesion one or more times daily over the course of a five-day application period. The test article was administered by way of a skin patch in accordance with the schedule shown in Table 69, below:

TABLE 69
Application schedule for Test Article 3 in a
subject diagnosed as having an eczema lesion

| Day | Application of Test Article 3 |
|---|---|
| Day 0 (baseline) | No application |
| Day 1 | Two applications: once in the morning and again |
| | in the evening |
| Day 2 | Two applications: once in the morning and again |
| | in the evening |
| Day 3 | One application |
| Day 4 | One application |
| Day 5 | One application |

On each day of the schedule shown in Table 69, the subject's eczema lesion was monitored by way of a visual assessment using the photographic techniques described in Examples 4 and 5, above. Particularly, the visual assessment included an evaluation of the discoloration of the subject's skin at the site of the lesion. The subject's condition was also monitored by way of a self-assessment, in which the subject evaluated their level of irritation, itchiness, inflammation, and sensitivity on a numerical scale. On this scale, a score of 10 represented the highest level of irritation, itchiness, inflammation, and sensitivity, and a score of 0 represented the lowest level of irritation, itchiness, inflammation, and sensitivity.

### Results

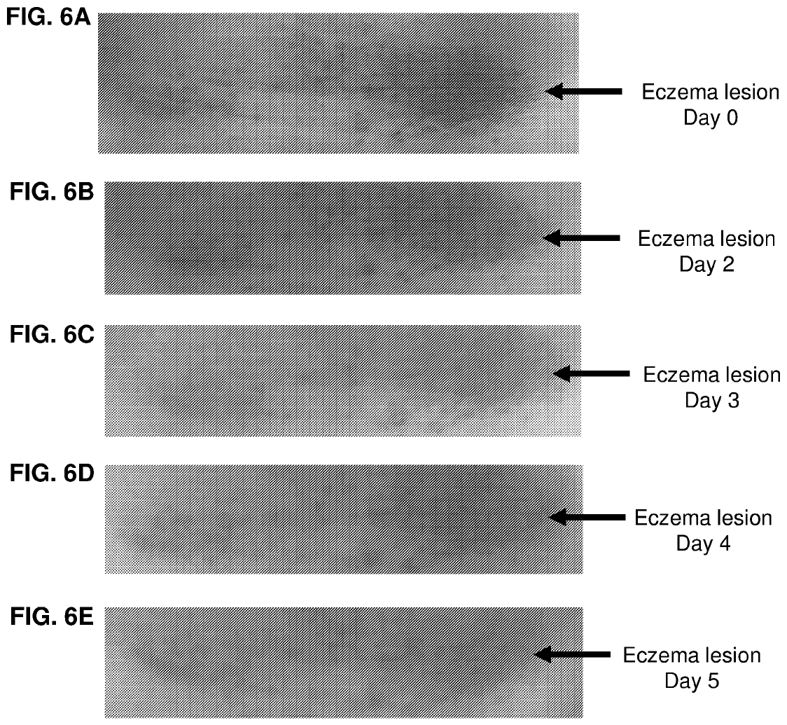

Throughout the course of the five-day application regimen described in Table 69, the subject exhibited a steady reduction in skin discoloration and an overall improvement in the skin's outward appearance at the application site. The results of the subject's visual assessment are show in FIGS. 6A-6E. As these figures demonstrate, the subject's discoloration steadily declined with increasing application of Test Article 3 throughout the five-day application period.

Additionally, during the five-day application period, the subject exhibited a consistent reduction in skin irritation, itchiness, inflammation, and sensitivity at the application site. Using the numerical self-assessment scale described above, at baseline, the subject's level of irritation, itchiness, inflammation, and sensitivity was 10. After four days of treatment, the subject's level of irritation, itchiness, inflammation, and sensitivity was between 0.5 and 1, using the same severity scale. After five days of treatment, the eczema was considered to be fully alleviated, as the subject reported a severity score of 0.

### Conclusion

Taken together, the results of this study demonstrate that compositions containing CBG can be used to improve the outward appearance of skin at the site of an eczema lesion, as well as to alleviate the irritation, itch, inflammation, and sensitivity associated with eczema.

### Example 7. Evaluating the Ability of CBG to Improve Mild to Moderate Outbreaks and Self-Perception

### Summary of the Clinical Study

This example describes the results of a human clinical study that was conducted with the aim of investigating the ability of CBG to improve the outward appearance of skin in subjects having acne. Particularly, the objective of this study was to evaluate the efficacy of three test articles and a placebo on mild to moderate acne on the face over a 4-week use period.

Subjects were healthy males and females aged 18-40 years with mild to moderate acne outbreaks.

Over the course of the study, subjects attended the testing facility on 4 separate visits, which included visits at baseline, on day 7, on day 14, and on day 28. Assessments were conducted at all timepoints. The assessments performed throughout the study included expert assessments made using the Investigator's Global Assessment (IGA) scale, as well as metrics such as lesion counts, redness, dryness/scaling, and swelling of the face. Additionally, subjects self-assessed the burning, stinging, itching, dryness/tightness of their face at these timepoints. Sebumeter readings were also taken at each timepoint. Subjects were given a self-perception questionnaire on days 7, 14, and 28. Professional photography images were taken at baseline and on days 7, 14, and 28.

The study was a single-blind, home-use design. Subjects were issued the test article to be used twice daily, once in the morning and again in the evening, during the 4-week test period. Study assessments were conducted according to Table 70 below.

TABLE 70
Summary of Study Assessments

| Study Timepoint | Baseline | Day 7 | Day 14 | Day 28 |
|---|---|---|---|---|
| Study Visit | 1 | 2 | 3 | 4 |
| Informed | ✓ | | | |
| Consent/Inclusion | | | | |
| Criteria | | | | |
| Visual Assessments | ✓ | ✓ | ✓ | ✓ |
| Sebumeter | ✓ | ✓ | ✓ | ✓ |
| Self-perceived | | ✓ | ✓ | ✓ |
| tolerability | | | | |
| Professional | ✓ | ✓ | ✓ | ✓ |
| photography images | | | | |
| Test Article distribution | D | | | C |
| (“D) and collection | | | | |
| (“C”) | | | | |
| Adverse Events | | ✓ | ✓ | ✓ |
| Subject Diary and | ✓ | | | |
| Information Sheet | | | | |

### Selection of Subjects

Subjects were selected in the same manner as described above in Example 5. An adequate number of subjects were enrolled so that 80 subjects would be expected to complete the study. The suitability of each subject to participate was confirmed prior to their acceptance in the study by completion and review of a study specific pre-study questionnaire.

### Materials and Methods

The three test articles and the placebo article included the ingredients shown in Table 71.

TABLE 71
Article Ingredients

| Article | Ingredients |
|---|---|
| Test Article 1 | Squalane, Caprylic/capric triglyceride, Cannabigerol - |
| | 4% cannabigerol w/w |
| Test Article 2 | Squalane, Caprylic/capric triglyceride, Cannabigerol - |
| | 2% cannabigerol w/w |
| Test Article 3 | Squalane, Caprylic/capric triglyceride, Cannabigerol - |
| | 1% cannabigerol w/w |
| Placebo Article | Squalane, Caprylic/capric triglyceride |

During the study, the test and placebo articles were applied in the form of 3-5 droplets over the face in the morning and evening. Test and placebo articles were applied after the subject cleansed the designated area.

### Study Procedure

### Pre-Assessment Acclimatization:

Subjects arrived to the testing facility at least 35 minutes prior to any study timepoints to ensure there was enough time to acclimatize prior to instrumental assessments being taken. Subjects sat resting in a temperature-controlled environment for a minimum of 30 minutes at a temperature of 22° C.±2° C. and at a relative humidity of 45%±5%.

### Baseline:

Potential subjects attended the testing facility where informed consent was be obtained, and eligibility was be verified. Once accepted, subjects were acclimatized for 30 minutes. Subjects then had baseline visual assessments taken of their face. Subjects then had a Sebumeter reading taken on their forehead. Following this, 30 subjects were randomly selected (10 from each test treatment group) to have clinical photography images taken of their face. Once taken, subjects were given the test article, an information sheet, and a diary to complete each time they used the product. Subjects were then be given a time slot for their next visit.

### Days 7, 14 and 28:

All subjects returned to the testing facility following 7, 14, and 28 days of using the product at home. Subjects were asked if there have been any changes to their health or medication since their previous visit. If there had been any, this was recorded and logged. Subjects were then asked to acclimatize for 30 minutes and then had the same visual and instrument assessments as the baseline visit. Subjects were then asked to rate the severity of the following attributes: burning, stinging, itching, dryness/tightness) of their skin from using the product. Subjects were then asked to complete a self-perception questionnaire on how they found using the product. Subjects that were selected for clinical photographs had these taken again.

### Assessments

A variety of assessments were conducted to determine the effects of test articles on subjects' skin. These assessments included grader evaluation, bioinstrumental measurements, and subjective perception. Grader assessments included a count of acne lesions, global assessment of acne, and counts of inflammatory and non-inflammatory acne lesions. At each test facility, the same trained grader conducted the grader assessments.

Acne lesions counts included non-inflammatory lesions consisting of open and closed comedones and inflammatory lesions consisting of papules, pustules, and nodular lesions. Acne lesions were counted at baseline, days 7, 14, and 28.

The global assessment of acne severity was measured according to Table 34 above in Example 5. Subjects were assigned a redness, swelling, and drying scores using the scales below in Tables 72-74:

TABLE 72
Redness Grading Scale

| Score | Description |
|---|---|
| 0 | No Redness |
| 1 | Barely perceptible redness |
| 2 | Slight redness |
| 3 | Moderate redness |
| 4 | Severe redness |

TABLE 73
Swelling Grading Scale

| Score | Description |
|---|---|
| 0 | None |
| 1 | Very slight (Barely perceptible) |
| 2 | Slight edema (edges of area well defined by definite raising) |
| 3 | Moderate edema (raised 1 mm) |
| 4 | Severe edema (raised >1 mm and extending beyond area of |
| | exposure) |

TABLE 74
Dryness/Scaling Grading Scale

| Score | Description |
|---|---|
| 0 | Extremely dry skin, more scale and pronounced separation |
| | of scale edges, some evidence of cracking |
| 1 | Severely dry skin; pronounced scaling, visible to the |
| | naked eye, definite uplifting of edges or scale sections- |
| | skin surface may have a whitish appearance |
| 2 | Moderately dry skin, fairly uniformly distributed scale |
| 3 | Slightly dry skin, occasional scale, not necessarily |
| | uniformly distributed |
| 4 | Smooth, no evidence of dry skin. |

Subjects were asked to rate the severity of the following attributes: burning, stinging, itching, and dryness/tightness of their skin after using the product at days 7, 14, and 28. Subjects rated the severity using the below scale in Table 75:

TABLE 75
Sensory Irritation Scale

| Stinging | Burning | Itching | Dryness/Tightness |
|---|---|---|---|
| 0 = None | 0 = None | 0 = None | 0 = None |
| 1 = Mild | 1 = Mild | 1 = Mild | 1 = Mild |
| 2 = Moderate | 2 = Moderate | 2 = Moderate | 2 = Moderate |
| 3 = Severe | 3 = Severe | 3 = Severe | 3 = Severe |

Subjects completed a Self-Perception Questionnaire (SPQ) to gauge the subject's perception of efficacy and tolerability on days 7, 14, and 28. Subjects determined their level of agreement with statements about the test article utilizing a five-point Likert scale.

### Results

85 subject were recruited and 83 completed the study. No adverse reactions were reported, and two subjects withdrew from the study due to personal reasons. The results of the study are summarized in the Tables below.

TABLE 76
Results of Statistical Analysis-Sebumeter

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 76.95 | 4.59 | | | 7.02E−01 |
| 1 | Day 7 | 67.54 | 4.15 | −12.23% | 1.75E−14 | 3.78E−06 |
| | Day 14 | 58.70 | 3.13 | −23.72% | 6.34E−15 | 1.97E−11 |
| | Day 28 | 48.07 | 3.65 | −37.53% | 6.38E−17 | 9.33E−15 |
| Test Article | Baseline | 76.18 | 5.09 | | | 8.87E−01 |
| 2 | Day 7 | 70.51 | 3.52 | −7.44% | 8.46E−05 | 8.87E−01 |
| | Day 14 | 61.98 | 3.17 | −18.64% | 5.79E−10 | 6.54E−05 |
| | Day 28 | 53.69 | 3.59 | −29.52% | 2.70E−12 | 4.26E−11 |
| Test Article | Baseline | 77.93 | 4.51 | | | 5.78E−13 |
| 3 | Day 7 | 73.65 | 2.80 | −5.49% | 3.44E−05 | 3.24E−01 |
| | Day 14 | 68.78 | 2.94 | −11.74% | 8.63E−10 | 1.86E−02 |
| | Day 28 | 62.68 | 3.64 | −19.57% | 6.19E−13 | 7.74E−07 |
| Placebo | Baseline | 76.36 | 4.88 | | | 4.91E−10 |
| Article | Day 7 | 77.01 | 4.89 | 0.85% | 2.63E−08 | |
| | Day 14 | 77.89 | 4.93 | 2.00% | 7.40E−11 | |
| | Day 28 | 79.02 | 5.15 | 3.49% | 3.33E−08 | |

TABLE 77
Results of Statistical Analysis-IGA Grade

| | | | | | | Between |
|---|---|---|---|---|---|---|
| | | | | | Within- | Treatment |
| | | | Std. | % Change | treatment | analysis vs |
| Test Site | Timepoint | Mean | Deviation | vs Baseline | analysis | Vehicle |
| Test Article | Baseline | 3.36 | 0.47 | | | 8.71E−01 |
| 1 | Day 7 | 2.02 | 0.50 | −39.86% | 1.06E−11 | 1.19E−10 |
| | Day 14 | 1.18 | 0.36 | −64.86% | 1.98E−17 | 1.01E−15 |
| | Day 28 | 0.71 | 0.25 | −78.76% | 2.65E−18 | 2.50E−16 |
| Test Article | Baseline | 3.24 | 0.44 | | | |
| 2 | Day 7 | 2.57 | 0.43 | −20.59% | 5.33E−11 | 3.30E−01 |
| | Day 14 | 1.95 | 0.47 | −39.71% | 9.34E−13 | 1.98E−08 |
| | Day 28 | 1.25 | 0.30 | −61.40% | 7.52E−12 | 1.35E−10 |
| Test Article | Baseline | 3.33 | 0.46 | | | 1.26E−14 |
| 3 | Day 7 | 2.86 | 0.45 | −14.29% | 3.79E−07 | 6.03E−01 |
| | Day 14 | 2.31 | 0.43 | −30.71% | 8.04E−13 | 8.52E−06 |
| | Day 28 | 1.88 | 0.38 | −43.57% | 3.23E−15 | 4.57E−08 |
| Placebo | Baseline | 3.40 | 0.49 | | | 3.87E−11 |
| Article | Day 7 | 3.67 | 0.40 | 7.69% | 2.09E−03 | |
| | Day 14 | 3.50 | 0.35 | 2.80% | 3.58E−01 | |
| | Day 28 | 3.57 | 0.40 | 4.90% | 1.10E−01 | |

TABLE 78
Results of Statistical Analysis-Total Lesion Count

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 12.95 | 4.39 | | | 7.48E−02 |
| 1 | Day 7 | 7.82 | 3.83 | −39.65% | 2.62E−07 | 5.39E−03 |
| | Day 14 | 5.05 | 2.89 | −61.05% | 5.73E−10 | 4.44E−06 |
| | Day 28 | 3.33 | 2.11 | −74.27% | 1.24E−10 | 4.22E−08 |
| Test Article | Baseline | 13.19 | 3.83 | | | |
| 2 | Day 7 | 10.52 | 3.83 | −20.22% | 8.31E−07 | 8.21E−02 |
| | Day 14 | 8.57 | 3.57 | −35.02% | 3.22E−11 | 6.93E−01 |
| | Day 28 | 7.65 | 3.10 | −42.00% | 4.38E−11 | 4.04E−02 |
| Test Article | Baseline | 15.48 | 19.55 | | | 2.35E−04 |
| 3 | Day 7 | 10.00 | 4.91 | −35.38% | 7.11E−06 | 3.49E−01 |
| | Day 14 | 8.62 | 4.48 | −44.31% | 1.23E−01 | 2.61E−01 |
| | Day 28 | 7.57 | 3.97 | −51.08% | 7.92E−02 | 1.21E−02 |
| Placebo | Baseline | 10.81 | 3.87 | | | 3.93E−04 |
| Article | Day 7 | 11.05 | 3.67 | 2.20% | 4.37E−01 | |
| | Day 14 | 11.24 | 3.81 | 3.96% | 1.19E−01 | |
| | Day 28 | 11.81 | 4.15 | 9.25% | 6.74E−03 | |

TABLE 79
Results of Statistical Analysis-Open Comedones

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 19.23 | 3.82 | | | 7.78E−01 |
| 1 | Day 7 | 13.27 | 3.71 | −30.97% | 6.82E−11 | 5.36E−06 |
| | Day 14 | 9.14 | 3.21 | −52.48% | 3.18E−14 | 3.87E−11 |
| | Day 28 | 6.14 | 2.67 | −68.05% | 7.17E−15 | 2.81E−13 |
| Test Article | Baseline | 17.10 | 3.39 | | | |
| 2 | Day 7 | 14.52 | 3.63 | −15.04% | 4.77E−11 | 1.24E−02 |
| | Day 14 | 12.67 | 3.28 | −25.91% | 1.08E−11 | 5.09E−05 |
| | Day 28 | 10.40 | 3.15 | −39.16% | 1.03E−15 | 5.27E−08 |
| Test Article | Baseline | 18.00 | 3.18 | | | 9.37E−10 |
| 3 | Day 7 | 15.90 | 2.96 | −11.64% | 2.48E−07 | 1.16E−01 |
| | Day 14 | 14.29 | 2.85 | −20.63% | 1.06E−11 | 7.27E−04 |
| | Day 28 | 13.43 | 2.73 | −25.40% | 2.48E−12 | 2.33E−06 |
| Placebo | Baseline | 19.76 | 3.28 | | | 2.37E−09 |
| Article | Day 7 | 20.29 | 3.65 | 2.65% | 1.26E−01 | |
| | Day 14 | 20.76 | 2.79 | 5.06% | 4.90E−03 | |
| | Day 28 | 21.29 | 2.76 | 7.71% | 1.99E−03 | |

TABLE 80
Results of Statistical Analysis-Closed Comedones

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 10.27 | 3.73 | | | 1.33E−01 |
| 1 | Day 7 | 6.27 | 2.43 | −38.94% | 2.36E−07 | 6.45E−05 |
| | Day 14 | 4.09 | 1.57 | −60.18% | 1.64E−09 | 8.07E−07 |
| | Day 28 | 3.10 | 1.58 | −69.87% | 1.39E−09 | 6.90E−07 |
| Test Article | Baseline | 8.95 | 3.22 | | | |
| 2 | Day 7 | 7.33 | 2.76 | −18.09% | 3.88E−08 | 4.61E−03 |
| | Day 14 | 6.24 | 2.68 | −30.32% | 1.19E−08 | 3.46E−04 |
| | Day 28 | 5.40 | 2.33 | −39.68% | 2.73E−08 | 2.61E−05 |
| Test Article | Baseline | 9.48 | 2.32 | | | 6.17E−05 |
| 3 | Day 7 | 8.43 | 1.91 | −11.06% | 7.81E−06 | 2.76E−02 |
| | Day 14 | 7.52 | 1.83 | −20.60% | 2.89E−08 | 5.01E−03 |
| | Day 28 | 6.95 | 1.80 | −26.63% | 1.90E−08 | 3.97E−04 |
| Placebo | Baseline | 12.48 | 5.10 | | | 1.19E−04 |
| Article | Day 7 | 12.52 | 5.17 | 0.38% | 8.67E−01 | |
| | Day 14 | 12.81 | 5.19 | 2.67% | 2.32E−01 | |
| | Day 28 | 12.52 | 5.36 | 0.38% | 8.58E−01 | |

TABLE 81
Results of Statistical Analysis-Papules

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 2.50 | 0.91 | | | 1.07E−01 |
| 1 | Day 7 | 1.00 | 0.76 | −60.00% | 4.66E−09 | 1.45E−07 |
| | Day 14 | 0.27 | 0.46 | −89.09% | 2.02E−11 | 1.52E−11 |
| | Day 28 | 0.10 | 0.30 | −96.19% | 5.56E−11 | 1.35E−10 |
| Test Article | Baseline | 2.57 | 1.03 | | | |
| 2 | Day 7 | 1.95 | 0.97 | −24.07% | 1.06E−04 | 1.48E−01 |
| | Day 14 | 1.38 | 0.80 | −46.30% | 1.60E−08 | 3.65E−04 |
| | Day 28 | 0.75 | 0.55 | −70.83% | 3.95E−09 | 1.08E−09 |
| Test Article | Baseline | 2.67 | 0.91 | | | 3.38E−08 |
| 3 | Day 7 | 2.19 | 0.75 | −17.86% | 3.79E−04 | 3.83E−01 |
| | Day 14 | 1.62 | 0.86 | −39.29% | 6.63E−11 | 2.73E−03 |
| | Day 28 | 1.05 | 0.67 | −60.71% | 2.69E−12 | 3.42E−07 |
| Placebo | Baseline | 2.95 | 0.97 | | | 5.43E−08 |
| Article | Day 7 | 3.24 | 1.14 | 9.68% | 5.54E−02 | |
| | Day 14 | 3.48 | 0.98 | 17.74% | 7.20E−04 | |
| | Day 28 | 3.10 | 0.94 | 4.84% | 4.80E−01 | |

TABLE 82
Results of Statistical Analysis-Pustules

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 1.41 | 1.01 | | | 2.67E−01 |
| 1 | Day 7 | 0.59 | 0.73 | −58.06% | 3.43E−05 | 3.63E−03 |
| | Day 14 | 0.14 | 0.35 | −90.32% | 2.50E−06 | 2.44E−05 |
| | Day 28 | 0.00 | 0.00 | −100.00% | 4.86E−06 | 2.83E−05 |
| Test Article | Baseline | 1.05 | 0.80 | | | |
| 2 | Day 7 | 0.67 | 0.66 | −36.36% | 2.21E−03 | 6.07E−02 |
| | Day 14 | 0.19 | 0.40 | −81.82% | 7.24E−06 | 6.13E−03 |
| | Day 28 | 0.15 | 0.37 | −85.68% | 6.01E−06 | 1.88E−04 |
| Test Article | Baseline | 1.05 | 1.02 | | | 1.71E−04 |
| 3 | Day 7 | 0.81 | 0.81 | −22.73% | 2.12E−02 | 6.92E−02 |
| | Day 14 | 0.48 | 0.60 | −54.55% | 9.47E−04 | 1.10E−02 |
| | Day 28 | 0.33 | 0.48 | −68.18% | 4.66E−04 | 3.21E−04 |
| Placebo | Baseline | 1.76 | 1.45 | | | 5.51E−05 |
| Article | Day 7 | 1.67 | 1.35 | −5.41% | 3.29E−01 | |
| | Day 14 | 1.90 | 1.48 | 8.11% | 1.86E−01 | |
| | Day 28 | 1.81 | 1.44 | 2.70% | 7.89E−01 | |

TABLE 83
Results of Statistical Analysis-Nodular Lesions

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 0.00 | 0.00 | | | N/A |
| 1 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Test Article | Baseline | 0.00 | 0.00 | | | N/A |
| 2 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Test Article | Baseline | 0.00 | 0.00 | | | N/A |
| 3 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Placebo | Baseline | 0.00 | 0.00 | | | N/A |
| Article | Day 7 | 0.00 | 0.00 | N/A | N/A | |
| | Day 14 | 0.00 | 0.00 | N/A | N/A | |
| | Day 28 | 0.00 | 0.00 | N/A | N/A | |

TABLE 84
Results of Statistical Analysis-Redness

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 2.14 | 0.56 | | | 8.29E−02 |
| 1 | Day 7 | 1.00 | 0.76 | −53.19% | 1.87E−10 | 2.70E−07 |
| | Day 14 | 0.30 | 0.43 | −86.17% | 3.48E−13 | 4.24E−13 |
| | Day 28 | 0.10 | 0.20 | −95.54% | 1.88E−13 | 2.60E−15 |
| Test Article | Baseline | 1.90 | 0.44 | | | 1.06E−10 |
| 2 | Day 7 | 1.33 | 0.46 | −30.00% | 2.55E−05 | 1.58E−03 |
| | Day 14 | 0.64 | 0.50 | −66.25% | 6.45E−12 | 1.06E−06 |
| | Day 28 | 0.33 | 0.41 | −82.94% | 1.58E−12 | 2.24E−10 |
| Test Article | Baseline | 2.05 | 0.50 | | | 1.06E−10 |
| 3 | Day 7 | 1.55 | 0.63 | −24.42% | 5.18E−05 | 3.08E−02 |
| | Day 14 | 0.98 | 0.46 | −52.33% | 7.76E−11 | 3.91E−05 |
| | Day 28 | 0.48 | 0.37 | −76.74% | 2.70E−13 | 1.28E−08 |
| Placebo | Baseline | 2.38 | 0.50 | | | 3.86E−12 |
| Article | Day 7 | 2.40 | 0.46 | 1.00% | 7.15E−01 | |
| | Day 14 | 2.43 | 0.53 | 2.00% | 4.28E−01 | |
| | Day 28 | 2.36 | 0.42 | −1.00% | 7.89E−01 | |

TABLE 85
Results of Statistical Analysis-Swelling

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 0.00 | 0.00 | | | N/A |
| 1 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Test Article | Baseline | 0.00 | 0.00 | | | N/A |
| 2 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Test Article | Baseline | 0.00 | 0.00 | | | N/A |
| 3 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Placebo | Baseline | 0.00 | 0.00 | | | N/A |
| Article | Day 7 | 0.00 | 0.00 | N/A | N/A | |
| | Day 14 | 0.00 | 0.00 | N/A | N/A | |
| | Day 28 | 0.00 | 0.00 | N/A | N/A | |

TABLE 86
Results of Statistical Analysis-Dryness/Scaling

| | | | | | Within- | Between |
|---|---|---|---|---|---|---|
| | | | Std. | % Change | treatment | Treatment |
| Test Site | Timepoint | Mean | Deviation | vs Baseline | analysis | analysis |
| Test Article | Baseline | 0.00 | 0.00 | | | N/A |
| 1 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Test Article | Baseline | 0.00 | 0.00 | | | N/A |
| 2 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Test Article | Baseline | 0.00 | 0.00 | | | N/A |
| 3 | Day 7 | 0.00 | 0.00 | N/A | N/A | N/A |
| | Day 14 | 0.00 | 0.00 | N/A | N/A | N/A |
| | Day 28 | 0.00 | 0.00 | N/A | N/A | N/A |
| Placebo | Baseline | 0.00 | 0.00 | | | N/A |
| Article | Day 7 | 0.00 | 0.00 | N/A | N/A | |
| | Day 14 | 0.00 | 0.00 | N/A | N/A | |
| | Day 28 | 0.00 | 0.00 | N/A | N/A | |

TABLE 87
Results of TopBox Self-perception Questionnaires - Week 1

| | Test | Test | Test | Placebo |
|---|---|---|---|---|
| Questions | Article 1 | Article 2 | Article 3 | Article |
| My skin appears clearer after using this | 100.00% | 90.48% | 71.43% | 28.57% |
| product. | | | | |
| I noticed a reduction in facial acne or | 100.00% | 85.71% | 61.90% | 19.05% |
| blemishes within “1” weeks of using this | | | | |
| product. | | | | |
| I noticed less breakouts after the first “1” weeks | 90.91% | 71.43% | 66.67% | 28.57% |
| using this product. | | | | |
| I noticed a reduction in redness within “1” | 86.36% | 85.71% | 76.19% | 38.10% |
| weeks of using this product. | | | | |
| The acne scars on my face are overall less | 72.73% | 90.48% | 52.38% | 19.05% |
| visible after using this product. | | | | |
| This product controls my breakout. | 95.45% | 90.48% | 71.43% | 28.57% |
| This product has reduced the appearance of | 90.91% | 71.43% | 66.67% | 23.81% |
| white heads. | | | | |
| This product has reduced the appearance of | 95.45% | 95.24% | 57.14% | 14.29% |
| black heads. | | | | |
| This product has reduced the size of my pores. | 95.45% | 90.48% | 61.90% | 23.81% |
| This product has reduced the appearance of | 95.45% | 80.95% | 52.38% | 19.05% |
| pustules. | | | | |
| The product reduced the redness of my skin. | 95.45% | 100.00% | 80.95% | 38.10% |
| This product has improved the overall | 86.36% | 85.71% | 52.38% | 38.10% |
| appearance of my skin. | | | | |
| This product has improved my overall skin | 77.27% | 85.71% | 52.38% | 28.57% |
| tone. | | | | |
| This product has made my skin look more | 81.82% | 90.48% | 71.43% | 23.81% |
| even. | | | | |
| This product helped brighten my complexion. | 72.73% | 95.24% | 61.90% | 19.05% |
| I would continue to use this product to help | 90.91% | 95.24% | 57.14% | 38.10% |
| with my acne. | | | | |
| I would consider replacing my usual products | 95.45% | 95.24% | 66.67% | 23.81% |
| with this product. | | | | |
| I feel more confident and happier with the use | 86.36% | 100.00% | 57.14% | 19.05% |
| of this product. | | | | |
| I would recommend this product to a friend or | 90.91% | 95.24% | 61.90% | 38.10% |
| family member. | | | | |
| I am satisfied with the performance of this | 95.45% | 100.00% | 71.43% | 38.10% |
| product. | | | | |

TABLE 88
Results of TopBox Self-perception Questionnaires - Week 2

| | Test | Test | Test | Placebo |
|---|---|---|---|---|
| Questions | Article 1 | Article 2 | Article 3 | Article |
| My skin appears clearer after using this | 95.45% | 90.48% | 76.19% | 47.62% |
| product. | | | | |
| I noticed a reduction in facial acne or | 95.45% | 90.48% | 80.95% | 23.81% |
| blemishes within “2” weeks of using this | | | | |
| product. | | | | |
| I noticed less breakouts after the first “2” | 90.91% | 85.71% | 90.48% | 42.86% |
| weeks using this product. | | | | |
| I noticed a reduction in redness within | 95.45% | 95.24% | 80.95% | 42.86% |
| “2” weeks of using this product. | | | | |
| The acne scars on my face are overall | 81.82% | 90.48% | 66.67% | 19.05% |
| less visible after using this product. | | | | |
| This product controls my breakout. | 95.45% | 90.48% | 90.48% | 52.38% |
| This product has reduced the | 95.45% | 76.19% | 71.43% | 28.57% |
| appearance of white heads. | | | | |
| This product has reduced the | 95.45% | 100.00% | 80.95% | 23.81% |
| appearance of black heads. | | | | |
| This product has reduced the size of my | 90.91% | 95.24% | 80.95% | 33.33% |
| pores. | | | | |
| This product has reduced the | 95.45% | 85.71% | 66.67% | 23.81% |
| appearance of pustules. | | | | |
| The product reduced the redness of my | 95.45% | 95.24% | 90.48% | 42.86% |
| skin. | | | | |
| This product has improved the overall | 95.45% | 95.24% | 90.48% | 52.38% |
| appearance of my skin. | | | | |
| This product has improved my overall | 81.82% | 85.71% | 76.19% | 19.05% |
| skin tone. | | | | |
| This product has made my skin look | 86.36% | 90.48% | 80.95% | 23.81% |
| more even. | | | | |
| This product helped brighten my | 77.27% | 95.24% | 80.95% | 23.81% |
| complexion. | | | | |
| I would continue to use this product to | 95.45% | 95.24% | 80.95% | 42.86% |
| help with my acne. | | | | |
| I would consider replacing my usual | 95.45% | 95.24% | 71.43% | 28.57% |
| products with this product. | | | | |
| I feel more confident and happier with | 86.36% | 100.00% | 76.19% | 23.81% |
| the use of this product. | | | | |
| I would recommend this product to a | 90.91% | 95.24% | 80.95% | 38.10% |
| friend or family member. | | | | |
| I am satisfied with the performance of | 95.45% | 100.00% | 85.71% | 42.86% |
| this product. | | | | |

TABLE 89
Results of TopBox Self-perception Questionnaires - Week 4

| | Test | Test | Test | Placebo |
|---|---|---|---|---|
| Questions | Article 1 | Article 2 | Article 3 | Article |
| My skin appears clearer after using this product. | 95.45% | 90.48% | 80.95% | 52.38% |
| I noticed a reduction in facial acne or blemishes | 95.45% | 90.48% | 90.48% | 38.10% |
| within “4” weeks of using this product. | | | | |
| I noticed less breakouts after the first “4” weeks | 95.45% | 90.48% | 95.24% | 42.86% |
| using this product. | | | | |
| I noticed a reduction in redness within “4” weeks | 95.45% | 95.24% | 90.48% | 47.62% |
| of using this product. | | | | |
| The acne scars on my face are overall less visible | 90.91% | 90.48% | 76.19% | 19.05% |
| after using this product. | | | | |
| This product controls my breakout. | 95.45% | 90.48% | 95.24% | 57.14% |
| This product has reduced the appearance of white | 95.45% | 80.95% | 90.48% | 33.33% |
| heads. | | | | |
| This product has reduced the appearance of black | 95.45% | 95.24% | 90.48% | 28.57% |
| heads. | | | | |
| This product has reduced the size of my pores. | 90.91% | 90.48% | 80.95% | 38.10% |
| This product has reduced the appearance of | 95.45% | 85.71% | 80.95% | 33.33% |
| pustules. | | | | |
| The product reduced the redness of my skin. | 95.45% | 95.24% | 95.24% | 47.62% |
| This product has improved the overall appearance | 95.45% | 95.24% | 95.24% | 57.14% |
| of my skin. | | | | |
| This product has improved my overall skin tone. | 95.45% | 85.71% | 76.19% | 19.05% |
| This product has made my skin look more even. | 95.45% | 95.24% | 80.95% | 23.81% |
| This product helped brighten my complexion. | 81.82% | 95.24% | 80.95% | 23.81% |
| I would continue to use this product to help with | 95.45% | 95.24% | 80.95% | 52.38% |
| my acne. | | | | |
| I would consider replacing my usual products with | 95.45% | 95.24% | 71.43% | 28.57% |
| this product. | | | | |
| I feel more confident and happier with the use of | 95.45% | 95.24% | 76.19% | 23.81% |
| this product. | | | | |
| I would recommend this product to a friend or | 95.45% | 95.24% | 80.95% | 42.86% |
| family member. | | | | |
| I am satisfied with the performance of this product. | 95.45% | 95.24% | 85.71% | 47.62% |

### Conclusion

Data from this study showed that test articles 1-3 significantly reduced sebum (Table 76), IGA grade (Table 77), lesion count (Table 78), open comedones (Table 79), closed comedones (Table 80), papules (Table 81), pustules (Table 82), and redness (Table 84) when compared to baseline grades p<0.005. Visual grades of nodular lesions (Table 83), dryness/scaling (Table 86) and Swelling (Table 85) all resulted in 0's at baseline and no changes were observed throughout the duration of the study.

Data obtained from self-perceived sensory ratings showed that all subjects perceived stinging, burning, itching, and dryness/tightness at a level of 0 at baseline and at all other study visits.

Analysis of the results achieved by test articles 1-3 as compared to the results observed for the vehicle-treated cohort was conducted by way of a between treatment analysis. Data collected from Sebumeter experiments as well as IGA grades showed there were no significant differences between grades for each cohort at baseline, thus validating the analysis. Data from post-use showed that all test articles 1-3 resulted in significant improvements in visual appearance grades compared to vehicle (p<0.05).

Data from the week 1 self-perception questionnaire showed that all test article-treated subjects exhibited at least a majority preference (>50%) in all questions asked. Test Article 1 and Test Article 2, in particular, showed greater amounts of high favorable mean percentages (>80%) (Table 87).

Data from the week 2 self-perception questionnaire showed Test Article 1 and Test Article 2 showed highly favorable (>80%) mean percentages in all but one question asked. Nonetheless, the question still showed a majority preference (>50%). Test Article 3 showed highly favorable responses (>80%) in the majority of questions asked. There were 6 responses which showed majority preference (>50%) (Table 88).

Data from the week 4 self-perception questionnaire showed that Test Article 1 and Test Article 2 resulted in highly favorable (>80%) mean percentages in all questions asked. Test Article 3 resulted in highly favorable (>80%) mean percentages in all but 4 questions asked. Nonetheless, these questions still showed a majority preference (>50%) (Table 89).

Taken together, the data generated from this study demonstrate that cosmetic compositions containing CBG, and particularly, cosmetic compositions having a final concentration of about 4% w/w CBG, are effectuate robust improvements in the outward appearance of skin in subjects experiencing mild-to-moderate outbreaks.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A cosmetic composition comprising cannabigerol (CBG).

2. A cosmetic composition comprising CBG and capric/caprylic triglyceride (CCT).

3. The cosmetic composition of claim 1 or 2, wherein the composition further comprises squalane.

4. A cosmetic composition comprising CBG and squalane.

5. The cosmetic composition of any one of claims 1-4, wherein the concentration of CBG in the cosmetic composition is from about 0.1% w/w to about 10% w/w.

6. The cosmetic composition of claim 5, wherein the concentration of CBG in the cosmetic composition is from about 0.5% w/w to about 5% w/w CBG.

7. The cosmetic composition of claim 6, wherein the concentration of CBG in the cosmetic composition is about 1% w/w.

8. The cosmetic composition of claim 6, wherein the concentration of CBG in the cosmetic composition is about 2% w/w.

9. The cosmetic composition of claim 6, wherein the concentration of CBG in the cosmetic composition is about 4% w/w.

10. The cosmetic composition of any one of claims 1-9, wherein the composition further comprises CCT.

11. The cosmetic composition of any one of claims 1-10, wherein the composition further comprises an additional cannabinoid.

12. The cosmetic composition of claim 11, wherein the additional cannabinoid is cannabidiol (CBD).

13. The cosmetic composition of any one of claims 1-12, wherein the composition further comprises one or more essential oils.

14. The cosmetic composition of claim 13, wherein the one or more essential oils comprise Lavandula angustifolia oil.

15. The cosmetic composition of claim 13 or 14, wherein the one or more essential oils comprise hemp oil.

16. The cosmetic composition of claim 15, wherein the hemp oil is Cannabis sativa seed oil.

17. The cosmetic composition of claim 13, wherein the one or more essential oils comprise Lavandula angustifolia oil and Cannabis sativa seed oil.

18. The cosmetic composition of any one of claims 1-17, wherein the composition further comprises one or more plant extracts.

19. The cosmetic composition of claim 18, wherein the plant extract is a flower, leaf, or stem extract.

20. The cosmetic composition of claim 19, wherein the plant extract is a Cannabis sativa flower, leaf, or stem extract.

21. The cosmetic composition of any one of claims 1-20, wherein the composition is formulated for topical administration to the skin of a human subject.

22. The cosmetic composition of claim 21, wherein the composition is a patch, liquid, gel, lotion, paste, cream, foam, serum, ointment, or stick.

23. A method of improving the outward appearance of the skin of a human subject, the method comprising administering to the subject the cosmetic composition of any one of claims 1-22.

24. A method of improving skin barrier function in a human subject, the method comprising administering to the subject the cosmetic composition of any one of claims 1-22.

25. A method of reducing transepidermal water loss in a human subject, the method comprising administering to the subject the cosmetic composition of any one of claims 1-22.

26. A method of reducing redness in the skin of a human subject, the method comprising administering to the subject the cosmetic composition of any one of claims 1-22.

27. A method of reducing flaking in the skin of a human subject, the method comprising administering to the subject the cosmetic composition of any one of claims 1-22.

28. A method of clearing acne in a human subject, the method comprising administering to the subject the cosmetic composition of any one of claims 1-22.

29. A method of reducing skin discoloration in a human subject having eczema, the method comprising administering to the subject the cosmetic composition of any one of claims 1-22.

30. A method of brightening the skin of a human subject, the method comprising administering to the subject the cosmetic composition of any one of claims 1-22.

31. A method of increasing skin luminosity in a human subject, the method comprising administering to the subject the cosmetic composition of any one of claims 1-22.

32. A method of reducing visual dullness in the skin of a human subject, the method comprising administering to the subject the cosmetic composition of any one of claims 1-22.

33. The method of any one of claims 23-32, wherein the cosmetic composition increases skin hydration, smooths skin, renews skin, and/or cleanses skin.

34. The method of any one of claims 23-33, wherein the cosmetic composition is administered to the subject topically.

35. The method of any one of claims 23-34, wherein the cosmetic composition is administered to the subject one or more times daily, optionally wherein the cosmetic composition is administered to the subject once daily, twice daily, or three times daily.

36. The method of claim 35, wherein the cosmetic composition is administered to the subject once daily.

37. The method of claim 35, wherein the cosmetic composition is administered to the subject twice daily, optionally wherein the cosmetic composition is administered to the subject once in the morning and once in the evening.

38. The method of claim 35, wherein the cosmetic composition is administered to the subject three times daily.

39. The method of any one of claims 35-38, wherein the cosmetic composition is administered to the subject for at least 7 continuous days.

40. The method of claim 39, wherein the cosmetic composition is administered to the subject for at least 14 continuous days.

41. The method of claim 40, wherein the cosmetic composition is administered to the subject for at least 28 continuous days.

42. A method of manufacturing the cosmetic composition of any one of claims 1-22, the method comprising admixing CBG in one or more carriers, diluents, or excipients.

43. The method of claim 42, wherein the CBG is further admixed with CCT.

44. The method of claim 42 or 43, wherein the CBG is further admixed with squalane.

45. The method of any one of claims 42-44, wherein the CBG is further admixed with an additional cannabinoid.

46. The method of claim 45, wherein the additional cannabinoid is CBD.

47. The method of any one of claims 42-46, wherein the CBG is further admixed with one or more essential oils.

48. The method of claim 47, wherein the one or more essential oils comprise Lavandula angustifolia oil.

49. The method of claim 47 or 48, wherein the one or more essential oils comprise hemp oil.

50. The method of claim 49, wherein the hemp oil is Cannabis sativa seed oil.

51. The method of claim 47, wherein the one or more essential oils comprise Lavandula angustifolia oil and Cannabis sativa seed oil.

52. The method of any one of claims 42-51, wherein the CBG is further admixed with one or more plant extracts.

53. The method of claim 52, wherein the plant extract is a flower, leaf, or stem extract.

54. The method of claim 53, wherein the plant extract is a Cannabis sativa flower, leaf, or stem extract.

55. The method of any one of claims 42-54, wherein the composition is formulated such that the final concentration of CBG in the composition is from about 0.1% w/w to about 10% w/w.

56. The method of claim 55, wherein the composition is formulated such that the final concentration of CBG in the composition is from about 0.5% w/w to about 5% w/w.

57. The method of claim 56, wherein the composition is formulated such that the final concentration of CBG in the composition is about 1% w/w.

58. The method of claim 56, wherein the composition is formulated such that the final concentration of CBG in the composition is about 2% w/w.

59. The method of claim 56, wherein the composition is formulated such that the final concentration of CBG in the composition is about 4% w/w

60. The method of any one of claims 42-59, wherein the cosmetic composition is formulated for topical administration to the skin of a human subject.

61. The method of claim 60, wherein the composition is a patch, liquid, gel, lotion, paste, cream, foam, serum, ointment, or stick.
